(19) United States
(12) Patent Application Publication
(10) Pub. No.: US 2007/0118054 A1
(43) Pub. Date: May 24, 2007

(54) METHODS AND SYSTEMS FOR MONITORING PATIENTS FOR CLINICAL EPISODES

(75) Inventors: Itzhak PINHAS, Holon (IL); Avner HALPERIN, Ramat Gan (IL); Arkadi AVERBOUKH, Rehovot (IL); Daniel H. LANGE, Kfar Vradim (IL); Yosef GROSS, Moshav Mazor (IL)

Correspondence Address: HESLIN ROTHENBERG FARLEY & MESITI PC, 5 COLUMBIA CIRCLE, ALBANY, NY, 12203, US

(73) Assignee: EarlySense Ltd., Ramat Gan (IL)

(21) Appl. No.: 11/552,872

(22) Filed: Oct. 25, 2006

Related U.S. Application Data
(60) Provisional application No. 60/731,934, filed on Nov. 1, 2005.
(60) Provisional application No. 60/784,799, filed on Mar. 23, 2006.
(60) Provisional application No. 60/843,672, filed on Sep. 12, 2006.

(51) Int. Cl. A61B 5/103

(52) U.S. Cl. 600/587

(57) ABSTRACT

Methods and systems for monitoring vital signs for the prediction and treatment of physiological ailments are provided. The methods and systems may be applied to the monitoring of a broad range of physiological ailments or “episodes,” including, but not limited to, asthma, hypoglycemia, coughing, edema, sleep apnea, labor, and REM sleep stages, among others. The methods employ sensors, for example, non-contact sensors, adapted to detect vital signs, such as heart rate or respiration rate, to produce signals that can be analyzed for trends, for deviations, or for comparison to prior conditions or criteria. The sensors may be positioned whereby the subject need not be viewed by the health care provider. Some methods and systems employ the use of “scores” based upon the combination of sensed vital signs or based upon a comparison of the vital signs to criteria.

### CROSS-REFERENCE TO RELATED APPLICATIONS

This application claims priority from pending provisional application 60/731,934 filed on Nov. 1, 2005; pending provisional application 60/784,799 filed on Mar. 21, 2006; and pending provisional application 60/843,672 filed on Sep. 12, 2006, the disclosures of which are incorporated by reference herein in their entirety.

The subject matter of the present application is also related to the subject matter of commonly-assigned U.S. Pat. No. 7,077,810, issued on Jul. 18, 2006; to the subject matter of commonly-assigned copending U.S. application U.S. application Ser. No. 11/446,281 filed on Jun. 2, 2006; and to the subject matter of commonly-assigned copending U.S. application Ser. No. 11/197,786 filed on Aug. 3, 2005, the disclosures of which are incorporated by reference herein in their entirety.

### FIELD OF THE INVENTION

The present invention relates generally to monitoring patients and predicting and monitoring abnormal physiological conditions, and specifically to methods and apparatus for monitoring abnormal physiological conditions by non-contact measurement and analysis of characteristics of physiological and/or physical parameters for the prediction and treatment of physiological episodes.

### BACKGROUND OF THE INVENTION

Chronic diseases are often expressed by episodic worsening of clinical symptoms. Preventive treatment of chronic diseases reduces the overall dosage of required medication and associated side effects, and lowers mortality and morbidity. Generally, preventive treatment should be initiated or intensified as soon as the earliest clinical symptoms are detected, in order to prevent progression and worsening of the clinical episode and to stop and reverse the pathophysiological process. Therefore, the ability to accurately monitor pre-episodic indicators increases the effectiveness of preventive treatment of chronic diseases.

Many chronic diseases cause systemic changes in vital signs, such as breathing and heartbeat patterns, through a variety of physiological mechanisms. For example, common respiratory disorders, such as asthma, chronic obstructive pulmonary disease (COPD), and cystic fibrosis (CF), are direct modifiers of breathing and/or heartbeat patterns. Other chronic diseases, such as diabetes, epilepsy, and certain heart conditions (e.g., congestive heart failure (CHF)), are also known to modify cardiac and breathing activity. In the case of certain heart conditions, such modifications typically occur because of pathophysiologies related to fluid retention and general cardiovascular insufficiency. Other signs such as coughing and sleep restlessness are also known to be of importance in some clinical situations.

Many chronic diseases induce systemic effects on vital signs. For example, some chronic diseases interfere with normal breathing and cardiac processes during wakefulness and sleep, causing abnormal breathing and heartbeat patterns.

Breathing and heartbeat patterns may be modified via various direct and indirect physiological mechanisms, resulting in abnormal patterns related to the cause of modification. Some respiratory diseases, such as asthma, and some heart conditions, such as CHF, are direct breathing modifiers. Other metabolic abnormalities, such as hypoglycemia and other neurological pathologies affecting autonomic nervous system activity, are indirect breathing modifiers.

Asthma is a chronic disease with no known cure. Substantial alleviation of asthma symptoms is possible via preventive therapy, such as the use of bronchodilators and anti-inflammatory agents. Asthma management is aimed at improving the quality of life of asthma patients. Asthma management presents a serious challenge to the patient and physician, as preventive therapies require constant monitoring of lung function and corresponding adaptation of medication type and dosage. However, monitoring of lung function is not simple, and requires sophisticated instrumentation and expertise, which are generally not available in the non-clinical or home environment.

Monitoring of lung function is viewed as a major factor in determining an appropriate treatment, as well as in patient follow-up. Preferred therapies are often based on aerosol-type medications to minimize systemic side-effects. The efficacy of aerosol type therapy is highly dependent on patient compliance, which is difficult to assess and maintain, further contributing to the importance of lung-function monitoring.

Asthma episodes usually develop over a period of several days, although they may sometimes seem to appear unexpectedly. The gradual onset of the asthmatic episode provides an opportunity to start countermeasures to stop and reverse the inflammatory process. Early treatment at the pre-episode stage may reduce the clinical episode manifestation considerably, and may even prevent the transition from the pre-clinical stage to a clinical episode altogether.

Two techniques are generally used for asthma monitoring. The first technique, spirometry, evaluates lung function using a spirometer, an instrument that measures the volume of air inhaled and exhaled by the lungs. Airflow dynamics are measured during a forceful, coordinated inhalation and exhalation effort by the patient into a mouthpiece connected via a tube to the spirometer. A peak-flow meter is a simpler device that is similar to the spirometer, and is used in a similar manner. The second technique evaluates lung function by measuring nitric-oxide concentration using a dedicated nitric-oxide monitor. The patient breathes into a mouthpiece connected via a tube to the monitor.

Efficient asthma management requires daily monitoring of respiratory function, which is generally impractical, particularly in non-clinical or home environments. Peak-flow meters and nitric-oxide monitors provide a general indication of the status of lung function. However, these monitoring devices have limited predictive value, and are used as during-episode markers. In addition, peak-flow meters and nitric-oxide monitors require active participation of the patient, which is difficult to obtain from many children and substantially impossible to obtain from infants.

Congestive heart failure (CHF) is a condition in which the heart is weakened and unable to circulate blood to meet the body's needs. The subsequent buildup of fluids in the legs, kidneys, and lungs characterizes the condition as congestive. The weakening may be associated with either the left, right, or both sides of the heart, with different etiologies and treatments associated with each type. In most cases, it is the left side of the heart which fails, so that it is unable to efficiently pump blood to the systemic circulation. The ensuing fluid congestion of the lungs results in changes in respiration, including alterations in rate and/or pattern, accompanied by increased difficulty in breathing and tachypnea.

Quantification of such abnormal breathing provides a basis for assessing CHF progression. For example, Cheyne-Stokes Respiration (CSR) is a breathing pattern characterized by rhythmic oscillation of tidal volume with regularly recurring periods of alternating apnea and hyperpnea. While CSR may be observed in a number of different pathologies (e.g., encephalitis, cerebral circulatory disturbances, and lesions of the bulbar center of respiration), it has also been recognized as an independent risk factor for worsening heart failure and reduced survival in patients with CHF. In CHF, CSR is associated with frequent awakening that fragments sleep, and with concomitant sympathetic activation, both of which may worsen CHF. Other abnormal breathing patterns may involve periodic breathing, prolonged expiration or inspiration, or gradual changes in respiration rate usually leading to tachypnea.

Fetal well-being is generally monitored throughout pregnancy using several sensing modalities, including ultrasonic imaging as a screening tool for genetic and developmental defects and for monitoring fetal growth, as well as fetal heartbeat monitoring using Doppler ultrasound transduction. It has been found that a healthy baby responds to activity by increased heart rate, similar to the way an adult's heart rate changes during activity and rest. Fetal heart rate typically varies between 80 and 250 heartbeats per minute, and accelerates with movement in a normal, healthy fetus. Lack of such variability has been correlated with a high incidence of fetal mortality when observed prenatally. In late stages of pregnancy, particularly in high-risk pregnancies, fetal heartbeat is commonly monitored on a regular basis to monitor fetal well-being and to identify initial signs of fetal distress, which usually result in active initiation of an emergency delivery. Current solutions to monitor fetal well-being are generally not suitable for home environments.

Ballistocardiography is the measurement of the recoil movements of the body which result from motion of the heart and blood in the circulatory system. Transducers are available which are able to detect minute movements of the body produced by the acceleration of the blood as it moves in the circulatory system. For example, U.S. Pat. No. 4,657,025 to Orlando, which is incorporated herein by reference, describes a device for sensing heart and breathing rates in a single transducer. The transducer is an electromagnetic sensor constructed to enhance sensitivity in the vertical direction of vibration produced on a conventional bed by the action of patient's heartbeat and breathing functions, and is described as achieving sufficient sensitivity with no physical coupling between the patient resting in bed and the sensor placed on the bed away from the patient.

The following patents and patent application publication, all of which are incorporated herein by reference, may also be of interest:

U.S. Pat. No. 7,077,810 to Lange et al.;

U.S. Pat. No. 4,657,026 to Tagg;

U.S. Pat. No. 5,235,989 to Zomer;

U.S. Pat. No. 5,957,861 to Combs;

U.S. Pat. No. 6,383,142 to Gavriely;

U.S. Pat. No. 6,436,057 to Goldsmith et al.;

U.S. Pat. No. 6,856,141 to Ariav;

U.S. Pat. No. 5,964,720 to Pelz;

US Patent application 20050119586 to Coyle et al.;

US Patent application 20060084848 to Mitchnick;

U.S. Pat. No. 6,984,207 to Sullivan; and

U.S. Pat. No. 6,375,621 to Sullivan.

An article by Shochat M et al., entitled, “PedemaTOR: Innovative method for detecting pulmonary edema at the pre-clinical stage,” undated, available at http://www.isramed.info/rsmm_rabinovich/pedemator.htm (which is incorporated herein by reference), describes an impedance monitor for pre-clinical detection of pulmonary edema. The impedance monitor measures “internal thoracic impedance,” which is roughly equal to lung impedance, by automatically calculating skin-electrode impedance and subtracting it from the measured transthoracic impedance.

The following articles, which are incorporated herein by reference, may also be of interest:
Alihanka J, et al., “A new method for long-term monitoring of the ballistocardiogram, heart rate, and respiration,” Am J Physiol Regul Integr Comp Physiol 240:384-392 (1981).
Bentur, L. et al., “Wheeze monitoring in children for assessment of nocturnal asthma and response to therapy,” Eur Respir J 21(4):621-626 (2003).
Chang, A. B. et al., “Cough, airway inflammation, and mild asthma exacerbation,” Archives of Disease in Childhood 86:270-275 (2002).
Hsu, J. Y., et al., “Coughing frequency in patients with persistent cough: assessment using a 24 hour ambulatory recorder,” Eur Respir J 7: 1246-1253 (1994).
Mack, D., et al., “Non-invasive analysis of physiological signals: NAPS: A low cost, passive monitor for sleep quality and related applications,” University of Virginia Health System (undated).
Korpas J, “Analysis of the cough sound: an overview,” Pulmonary Pharmacology 9:261-268 (1996).
Thorpe, C.; Toop, L.; and Dawson, K., “Towards a quantitative description of asthmatic cough sounds,” Eur. Respir. J, 1992, 5, 685-692.
Hirtum, A.; Berckmans, D.; Demuynck, K.; and Compernolle, D., “Autoregressive Acoustical Modelling of Free Field Cough Sound,” Proc. International Conference on Acoustics, Speech and Signal Processing, volume I, pages 493-496, Orlando, U.S.A., May 2002.
Piirila, P., et al., “Objective assessment of cough,” Eur Respir J 8:1949-1956 (1995).
Salmi, T., et al., “Long-term recording and automatic analysis of cough using filtered acoustic signals and movements on static charge sensitive bed,” Chest 94:970-975 (1988).
Salmi, T., et al., “Automatic analysis of sleep records with static charge sensitive bed,” Electroencephalography and Clinical Neurophysiology 64:84-87 (1986).
Stegmaier-Stracca, P. A., et al., “Cough detection using fuzzy classification,” Symposium on Applied Computing, Proceedings of the 1995 ACM Symposium on Applied Computing, Nashville, Tenn., United States, pp. 440-444 (1995).
Van der Loos, H. F. M., et al., “Unobtrusive vital signs monitoring from a multisensor bed sheet,” RESNA'2001, Reno, Nev., Jun. 22-26, 2001.
Waris, M., et al., “A new method for automatic wheeze detection,” Technol Health Care 6(1):33-40 (1998).
Katz, M.; Gill, P.; and Newman, R., “Detection of preterm labor by ambulatory monitoring of uterine activity: a preliminary report”, Obstetrics & Gynecology 1986;68:773-778.
“British Guideline on the Management of Asthma: A national clinical guideline,” British Thoracic Society, Scottish Intercollegiate Guidelines Network, Revised edition April 2004.
Brenner, B. E., et al., “The clinical presentation of acute asthma in adults and children,” In Brenner, B E, ed. Emergency Asthma (New York: Marcel Dekker, 1999:201-232).
Baren, et al., “Current concepts in the ED treatment of pediatric asthma,” Respiratory Medicine Consensus Reports (Thomson American Health Consultants, Dec. 28, 2003).
“Managing Asthma,” KidsHealth website, (kidshealth.org/parent/medical/lungs/asthma_mgmt.html).
“Signs and symptoms of asthma,” Indian Chest Society (Mumbai, India) (http://www.indianchestsociety.org/symptomsofasthma.htm).
“Breathing easier with asthma,” Intermountain Health Care Clinical Education Services (http://www.ihc.com/xp/ihc/documents/clinical/101/3/1/asthma_breathe.pdf).
“Medical Mutual clinical practice guidelines for asthma: 2004,” Medical Mutual (Cleveland, Ohio) (http://www.medmutual.com/provider/pdf/resources/asthma4.pdf).
“Peak flow learning center,” National Jewish Medical and Research Center (http://www.njc.org/diseaseinfo/diseases/asthma/living/tools/peak/index.aspx).
Mintzer, R., “What the teacher should know about asthma attacks,” Family Education Network (http://www.familyeducation.com/article/0, 1120,65-415,00.html).
“‘Does my child have asthma?’,” Solano Asthma Coalition, American Lung Association of the East Bay (http://www.alaebay.org/misc_pdf/solano_asthma_coalition _child_asthma.pdf).
Poteetm, J. “Asthma” (http://www.nku.edu/˜rad350/asthmajp.html).
Plaut, T., “Tracking and treating asthma in young children,” J Respir Dis Pediatrician 5(2):67-72 (2003).
Fitzpatrick, M. F., et al., “Snoring, asthma and sleep disturbances in Britain: A community based survey,” Eur Respiratory J 1993;6:531-5,
Jobanputra, P., et al., “Management of acute asthma attacks in general practice,” Br J Gen Pract 1991;41:410-3,
Lim, T. O., et al., “Morbidity associated with asthma and audit of asthma treatment in outpatient clinics,” Singapore Med J 1992;33:174-6,
Madge, P. J., et al., “Home nebuliser use in children with asthma in two Scottish Health Board Areas,” Scott Med J 1995:40:141-3.
Watanabe, T., et al., “Noncontact Method for Sleep Stage Estimation,” IEEE Transactions on Biomedical Engineering, No 10, Vol. 51, October 2004.
Yongjoon, C., et al., “Air mattress sensor system with balancing tube for unconstrained measurement of respiration and heart beat movements”, 2005 Physiol. Meas. 26 413-422

United States Patent Application Publication 2005/0192508 to Lange et al. and PCT Patent Publication WO 2005/074361 to Lange et al., which are assigned to the assignee of the present patent application and are incorporated herein by reference, describe a method for predicting an onset of a clinical episode. The method includes sensing breathing of a subject, determining at least one breathing pattern of the subject responsively to the sensed breathing, comparing the breathing pattern with a baseline breathing pattern, and predicting the onset of the episode at least in part responsively to the comparison. Other embodiments are also described.

The inclusion of the foregoing references in this Background section does not imply that they constitute prior art or analogous art with respect to the invention disclosed herein.

### SUMMARY OF THE INVENTION

Aspects of the present invention provide many methods and systems for monitoring patients for the occurrence or recurrence of a physiological event, for example, a chronic illness or ailment, that can assist the patient or healthcare provider in treating the ailment or mitigating the effects of the ailment. By means of automated sensors and electronic signal processing, aspects of the invention detect vital, and not so vital, signs to detect and characterize the onset of a physiological event and, in some aspects, treat the event, for example, with therapy or medication.

In some embodiments, the present invention includes methods and systems for monitoring many kinds of medical conditions, for example, chronic medical conditions, and include the use a motion acquisition module, a pattern analysis module, and an output module. The chronic medical condition monitored may be any medical condition, for example, asthma, apnea, insomnia, congestive heart failure, hypoglycemia, and the like, for example, as described herein. The methods, systems, and apparatuses described herein may be adapted to perform one or more of the methods described herein, as appropriate. For example, a control unit of the systems and apparatuses may be adapted to carry out one or more steps of the methods (such as analytical steps), and/or the sensor of the apparatuses may be adapted to carry out one or more of the sensing steps of the methods.

Embodiments of the invention include methods and systems for simultaneous measurement of heart rate and respiration rate including calculation of the ratio of the heart rate signal amplitude to the respiration rate signal amplitude and comparing said ratio with a criterion to determine whether the heart rate signal is valid.

Other embodiments include methods and systems for monitoring of patients in bed including measurement of body movement signal, calculation of standard deviation of that signal and comparing said standard deviation to a criterion in order to determine whether there has been a body posture change.

Other embodiments include methods and systems for measuring palpitations during sleep, for example, in a contact-less manner; methods and systems for monitoring clinical parameters of patients for long durations of time and correlating changes in clinical parameters with clinical and non-clinical parameters and/or events; and methods and systems for monitoring clinical parameters over a long period of time to identify long term processes in the development of chronic conditions, for example, employing a contact-less sensor.

Other embodiments of the invention include methods and systems for monitoring chronic patients including monitoring clinical parameters in a contact-less manner, identifying a change in the baseline of the clinical parameters and correlating that change with a change in therapeutic regime; methods and systems for contact-less monitoring of respiration patterns including identification of augmented breaths or deep inspirations; and methods and systems for monitoring asthma patients including monitoring clinical parameters and identifying the use of a medication through a change in a clinical parameter.

Other embodiments of the invention include methods and systems for monitoring a clinical condition including monitoring clinical parameters during sleep and identifying sleep stages and comparing the clinical parameters in at least one sleep stage to baseline clinical parameters for that sleep stage. The methods and device for identifying sleep stages may include a motion acquisition module, a pattern analysis module and an output module, as described below.

Other embodiments of the invention include methods and systems for monitoring a clinical condition including monitoring a patient while in bed, identifying when the patient falls asleep, and measuring a clinical parameter after the patient falls asleep and comparing it to a baseline for the clinical parameter in sleep.

Further embodiments of the invention include methods and systems for measuring respiration rate or expiration/inspiration ratio using heart beat patterns; methods and systems for determining a vagal nerve stimulation treatment protocol for a patient, including analyzing a respiration pattern of the patient; methods and systems for monitoring of premature babies, that is, preemies, for example, contact-less monitoring of premies; and methods and systems for calculating a clinical score for a chronic condition comprising measurement of multiple clinical parameters during sleep.

Other embodiments of the invention include methods and systems for enabling the use of risky therapeutic regimes including contact-less periodic monitoring of clinical parameters to monitor treatment effectiveness or occurrence of side effects; methods and systems for monitoring clinical parameters in bed including a mechanical sensor placed on top of the bed mattress without need for contacting the patient or the patient's clothes; and methods and systems for identifying whether a chronic patient is close to his optimal clinical parameter baseline including providing the patient with stronger medication than he or she is normally given, and monitoring the patient for improvement in clinical parameters.

Further embodiments of the invention include methods and systems for identifying parameters affecting a group of patients affected by a common external parameter by monitoring the condition of the group of patients and correlating their clinical results.

Other embodiments of the invention include methods and systems for measuring heart rate, including demodulating a high frequency spectrum of a ballistocardiography signal.

In some embodiments, the present invention includes methods and systems for monitoring sleeping subjects and identifying one or more sleep stages, for example, REM sleep stages. These methods and systems may include the use of a motion acquisition module, a pattern analysis module, and an output module. In one aspect, the sleep stage identified is REM sleep, for example, by analyzing a breathing rate variability (BRV) signal to identify REM sleep. The methods and systems for identifying one or more sleep stages may be practiced without contacting or viewing the subject. In one aspect, methods and systems are provided for monitoring or predicting deteriorations of chronic conditions by analyzing clinical parameters during REM sleep.

Further embodiments of the invention include methods and systems for identifying edema in a subject without contacting or viewing the subject; methods and systems for evaluating the multiple body motion parameters of a subject during sleep without contacting or viewing the subject; and methods and systems for identifying periodic breathing or Cheyne-Stokes respiration using signal demodulation analysis.

Further embodiment of the invention include methods and systems for identifying pulmonary edema, for example, by measuring an angle of the patient's body while the patient is asleep.

Other embodiments of the invention include methods and systems for identifying hypoglycemia in a patent and methods for detecting and treating hypoglycemia in a patient automatically, for example, by using a non-contact sensor. These methods and systems may include one or more alarms that advise the patient or the healthcare provider when a hypoglycemic episode is about to occur or is occurring. The methods and systems may include a motion acquisition module, a pattern analysis module, and an output module, as discussed below.

Still further embodiments of the invention include methods and systems for identifying drug efficacy in a patient, for example, without receiving compliance from the patient; and methods and devices for informing a patient of a prescribed limitation of patient activity, for example, based upon an automatic monitoring of the patient's condition.

In some embodiments, the present invention provides methods and systems for identifying cough events. The methods and systems may include a motion acquisition module, a pattern analysis module, and an output module for identifying cough events. In one aspect, the methods and systems identify cough by identifying frequency change in the acoustic signal; for example, the methods and systems may be adapted to analyze a recorded and digitized acoustic signal and identify cough from frequency criteria. In another aspect, the methods and systems for identifying cough identify a pattern of change in the frequency of the acoustic signal during the cough event. In still another aspect, the methods and systems are adapted to differentiate between cough of a person with edema and cough of a person without edema.

In some embodiments, the present invention includes systems and methods for monitoring uterine contractions, for example, for predicting the onset of preterm labor. Such systems may include a motion acquisition module, a pattern analysis module, and an output module. Aspects of this invention may be used for monitoring uterine contractions and predicting the onset of preterm labor, for example, without viewing or touching the pregnant woman's body, for instance, without obtaining compliance from the woman.

In some embodiments, the present invention includes methods and systems for monitoring or predicting apnea events, for example, during sleep. These methods and systems may include use of a motion acquisition module, a pattern analysis module, and an output module. In one aspect, the methods and systems may be used for monitoring a patient's clinical parameters during sleep and identifying and predicting the onset of apnea events, and activating immediate treatment.

In some embodiments, the present invention includes methods and systems for monitoring sexual intercourse. These methods and systems may include the use of a motion acquisition module, a pattern analysis module, and an output module. In one aspect, the methods and systems may be used for monitoring sexual intercourse, for example, without viewing or touching the patient's body, for the purpose of, for example, treating premature ejaculation.

Another embodiment of the invention is method for detecting an onset of a hypoglycemia episode in a subject, the method comprising monitoring one or more critical parameters for hypoglycemia, for example, without contacting the subject; detecting a variation of at least one of the critical parameters; and activating an alarm when at least one of the critical parameters deviates from an accepted value. In one aspect, the critical parameters comprise at least one of respiration rate, heart rate, occurrence of palpitations, restlessness, and tremor.

Another embodiment of the invention is an apparatus for detecting an onset of a hypoglycemia episode in a subject, the apparatus comprising at least one sensor adapted to monitor one or more critical parameters for hypoglycemia, for example, without contacting or viewing the subject; an analyzer adapted to detect a variation of at least one of the critical parameters; and means for activating an alarm when at least one of the critical parameters deviates from an accepted value.

Another embodiment of the invention is method for detecting a cough in a subject, the method comprising sensing an audio signal near the subject, for example, without contacting the subject; and analyzing the sensed audio signal and identifying frequency changes in the audio signal, for example, variations in the time-frequency characteristic of the audio signal, to identify the cough. In one aspect, analyzing the audio signal comprises identifying frequency changes in the audio signal to identify the cough.

Another embodiment of the invention is a an apparatus for detecting a cough in a subject, the apparatus comprising an electronic audio signal detector adapted to sense an audio signal, for example, without contacting the subject; and a signal analyzer adapted to analyze the sensed audio signal and identify frequency changes in the audio signal, for example, variations time-frequency characteristic of the audio signal, to identify the cough. In one aspect, the analyzer is further adapted to select a time interval in response to a least one of energy of the audio signal and amplitude of the audio signal.

Another embodiment of the invention is an apparatus for detecting a cough in a subject, the apparatus comprising an audio signal sensor, for example, near the subject; a motion sensor adapted to sense a motion of the subject without contacting the subject and generate a motion signal corresponding to the sensed motion; a signal analyzer adapted to analyze the audio signal and the motion signal to identify the cough.

Another embodiment of the invention is a method for detecting a cough in a subject, the method comprising sensing an audio signal near the subject; sensing a motion of the subject, for example, without contacting or viewing the subject, and generating a motion signal corresponding to the sensed motion; analyzing the audio signal and the motion signal to identify the cough.

Another embodiment of the invention is an apparatus for detecting a cough in a subject, the apparatus comprising an audio signal sensor; a motion sensor adapted to sense a motion of the subject, for example, without contacting or viewing the subject, and generate a motion signal corresponding to the sensed motion; and a signal analyzer adapted to analyze the audio signal and the motion signal to identify the cough.

Another embodiment of the invention is a method for detecting edema in a subject, the method comprising: providing a plurality of mechanical sensors, for example, weight sensors, each mechanical sensor adapted to sense a mechanical signal of a part of the body of the subject, for example, without contacting the subject; sensing a plurality of mechanical signals from the plurality of sensors; and analyzing the plurality of mechanical signals to determine the presence of edema. In one aspect, analyzing the plurality of mechanical signals comprises detecting mechanical signal distribution of the subject to determine the presence of edema.

Another embodiment of the invention is a system for detecting edema in a subject, the system comprising a plurality of mechanical sensors, each sensor adapted to sense a mechanical signal of a part of the body of the subject, for example, without contacting the subject, and produce a plurality of mechanical signals from the plurality of sensors; and a signal analyzer adapted to analyze the plurality of mechanical signals to determine the presence of edema. The mechanical sensors may be pressure sensors or accelerometers, among other sensors.

Another embodiment of the invention is a method of detecting an onset of apnea, the method comprising sensing motion of a subject, for example, without contacting the subject, the motion comprising motions related to at least breathing, and generating a signal corresponding to the sensed motion; extracting a breathing-related signal from the sensed motion signal corresponding to the breathing of the subject; and analyzing the breathing-related signal to predict the onset of apnea. In one aspect, the method may also comprise extracting and analyzing a heart rate signal. In one aspect, analyzing comprises detecting an increase in amplitude of at least one of the breathing-related signal and the heartbeat-related signal to detect the onset of apnea.

Another embodiment of the invention is a system for detecting an onset of apnea, the system comprising at least one sensor adapted to sense motion of a subject, for example, without contacting the subject, the motion comprising motions related to at least breathing, and generate a signal corresponding to the sensed motion; and an analyzer adapted to extract a breathing-related signal from the sensed motion signal corresponding to the breathing of the subject, and analyze the breathing-related signal to predict the onset of apnea. In one aspect, the analyzer may also extract a heartbeat signal from the sensed motion signal and analyze the heartbeat signal to predict the onset of apnea.

Another embodiment of the invention is a method of detecting the onset of apnea, the method comprising sensing an audio signal, for example, near the subject; sensing breathing of the subject, for example, without contacting the subject, and generating a breathing-related signal corresponding to the sensed breathing; analyzing the audio signal and the breathing-related signal to detect the onset of apnea.

Another embodiment of the invention is an apparatus for detecting the onset of apnea, the apparatus comprising an audio sensor adapted to generate an audio signal; at least one senor adapted to sense breathing of the subject, for example, without contacting the subject, and generate a breathing-related signal corresponding to the sensed breathing; and an analyzer adapted to analyze the audio signal and the breathing-related signal to detect the onset of apnea.

Another embodiment of the invention is a method for detecting uterine contractions in a pregnant woman, the method comprising sensing motion of the woman, for example, without contacting the woman, and generating a signal corresponding to the sensed motion; and analyzing the signal to detect presence of labor contractions. In one aspect, sensing motion of the women comprises sensing motion in the lower abdomen, the pelvis, and the upper abdomen of the women and generating a motion-related signal for the lower abdomen, the pelvis, and the upper abdomen to detect the presence of labor contractions.

Another embodiment of the invention is an apparatus for detecting uterine contractions in a pregnant woman, the apparatus comprising at least one motion sensor adapted to detect motion of the woman, for example, without contacting the woman, and generate at least one signal corresponding to the sensed motion; and a signal analyzer adapted to analyze the at least one signal to detect the presence of labor contractions.

Another embodiment of the invention is a method for identifying rapid eye movement (REM) sleep in a subject, the method comprising sensing breathing of the subject, for example, without contacting the subject, and generating a breathing-related signal corresponding to the sensed breathing; and analyzing the breathing-related signal to detect an occurrence of REM sleep.

Another embodiment of the invention is an apparatus for identifying rapid eye movement (REM) sleep in a subject, the apparatus comprising at least one sensor adapted to sense breathing of the subject, for example, without contacting the subject, and generate a breathing-related signal corresponding to the sensed breathing; and a signal analyzer adapted to analyze the breathing-related signal to detect an occurrence of REM sleep.

Another embodiment of the invention is a method for simultaneous measurement of heart rate and respiration rate of a subject, the method comprising sensing motion of the subject and generating a sensed motion signal responsive to the sensed motion; determining a heart beat related signal from the sensed motion signal; determining a first breathing rate related signal from the heart beat related signal; determining a second breathing rate related signal directly from the sensed motion signal; and comparing the first breathing rate related signal with the second breathing rate related signal to determine validity of the heart rate related signal.

Another embodiment of the invention is a system for simultaneous measurement of heart rate and respiration rate of a subject, the system comprising at least one motion sensor adapted to detect motion of the subject and generate a sensed motion signal responsive to the sensed motion; and a signal analyzer adapted to determine a heart beat related signal from the sensed motion signal, adapted to determine a first breathing rate related signal from the heart beat related signal, adapted to determine a second breathing rate related signal directly from the sensed motion signal, and adapted to compare the first breathing rate related signal with the second breathing rate related signal to determine validity of the heart rate related signal.

Another embodiment of the invention is a method for monitoring change in body position of a subject, the method comprising sensing motion of the subject, for example, without contacting the subject, and generating a sensed motion signal representative of the sensed motion; determining a variation of the sensed motion signal; and comparing the variation to a criterion to determine whether the subject changed body position.

Another embodiment of the invention is system for monitoring change in body position of a subject, the system comprising at least one sensor adapted to sense motion of the subject, for example, without contacting the subject, and generate a motion signal representative of the sensed motion; means for determining a variation of the motion signal; and means for comparing the variation to a criterion to determine whether the subject changed body position.

Another embodiment of the invention is a method for monitoring a subject, the method comprising sensing a plurality of clinical parameters of the subject, for example, without contacting the subject, and generating a plurality of clinical parameter signals representative of the plurality of clinical parameters; combining the plurality of the clinical parameter signals, and analyzing the combined clinical parameter signals to monitor or predict a clinical event.

Another embodiment of the invention is a method for monitoring the condition of a subject having a respiratory illness, the method comprising determining a plurality of parameters for the subject over at least three days, for example, without contacting the subject; evaluating a respiratory illness score, S(D), based upon the parameters for each day, D; and comparing the respiratory illness score, S(D), for day D to the score of the subject for at least one day prior to day D to determine relative condition of the subject. In one aspect, respiratory illness score may be evaluated by the equation

$$S ( D ) = \frac{{\sum }_{1}^{n} CiPi}{N}$$

where Pᵢ is at least one of the pluralities of parameters; Cᵢ is a constant associated with one of the plurality of parameters Pᵢ; N a constant associated with the constant Cᵢ; and n is the number of parameters. The respiratory illness may be asthma or chronic obstructive pulmonary disease (COPD), among other respiratory illnesses.

Another embodiment of the invention is a method for detecting a respiration rate from a heart rate of a subject, the method comprising sensing a heart rate of the subject, for example, without contacting the subject, and generating a signal representative of the heart rate; and analyzing the heart rate signal to determine the respiration rate of the subject.

Another embodiment of the invention is a method for monitoring an onset of a respiratory episode in a subject, the method comprising sensing a plurality of respirations of the subject and generating a plurality of respiration signals corresponding to the plurality of respirations; combining the plurality of respiration signals to provide a characteristic respiration parameter of the subject; and predicting the onset of the respiratory episode from the characteristic respiration parameter. In one aspect, the combining the plurality of respiration signals to provide a characteristic respiration parameter comprises calculating a respiration score from the plurality of respiration signals.

Another embodiment of the invention is a method for determining restlessness of a subject, the method comprising sensing motion of the subject with a motion sensor which produces a electrical signal responsive to the sensed motion; filtering the sensed signal to generate an signal corresponding to heart rate of the subject; filtering the sensed signal to generate an signal corresponding the breathing rate of the subject; and comparing the signal corresponding to the heart rate with the signal corresponding to the breathing rate to determine a level of restlessness of the subject.

Another embodiment of the invention is a method for determining restlessness of a subject, the method comprising sensing motion of the subject with a motion sensor which produces a signal responsive to the sensed motion; determining a variation of the sensed motion signal over at least two time epochs; comparing the variation between the at least two time epochs to determine restlessness of the subject.

In some aspects of the invention, methods and systems are provided for identifying respiratory depression, for example, without touching or viewing the patient's body; for identifying and monitoring teeth gritting in sleep; for monitoring and predicting changes in blood oxygen level; and for monitoring the change in fluid distribution in a patient's body during sleep.

In some aspects of the invention, methods and systems are provided for measurement of heart rate, for example, by demodulating a high frequency spectrum of a ballistocardiography signal; and methods and systems are provided for evaluating the multiple body motion parameters of a subject during sleep, for example, without contacting or viewing the subject.

In some embodiments of the present invention, methods and systems for monitoring chronic medical conditions is provided. These methods and systems may include providing a motion acquisition module, a pattern analysis module, and an output module.

In some embodiments of the present invention, the systems described hereinabove are adapted to perform one or more of the methods described hereinabove, as appropriate. For example, a control unit of the systems may be adapted to carry out one or more steps of the methods (such as analytical steps), and/or a sensor of the systems may be adapted to carry out one or more of the sensing steps of the methods.

### BRIEF DESCRIPTION OF THE DRAWINGS

The subject matter, which is regarded as the invention, is particularly pointed out and distinctly claimed in the claims at the conclusion of this specification. The foregoing and other objects, features, and advantages of the invention will be readily understood from the following detailed description of aspects of the invention taken in conjunction with the accompanying drawings in which:

FIG. 1 is a schematic illustration of a system for monitoring a chronic medical condition of a subject in accordance with an embodiment of the present invention.

FIG. 2 is a schematic block diagram illustrating components of control unit of the system of FIG. 1 in accordance with an embodiment of the present invention.

FIG. 3 is a schematic block diagram illustrating a breathing pattern analysis module of the control unit of FIG. 2, in accordance with an embodiment of the present invention.

FIGS. 4A, 4B, and 4C are graphs illustrating the analysis of motion signals, measured in accordance with an embodiment of the present invention.

FIG. 5 is a graph illustrating breathing rate patterns of a chronic asthma patient, measured during an experiment conducted in accordance with an embodiment of the present invention.

FIGS. 6 and 7 are graphs of exemplary baseline and measured breathing rate and heart rate nighttime patterns, respectively, measured in accordance with an embodiment of the present invention.

FIGS. 8A and 8B are graphs showing different frequency components of a motion signal, in accordance with an embodiment of the present invention.

FIG. 9 includes graphs showing several signals in time and corresponding frequency domains, in accordance with an embodiment of the present invention.

FIGS. 10A, 10B, and 10C are graphs showing frequency spectra, measured in accordance with an embodiment of the present invention.

FIG. 11 includes graphs showing combined and decomposed maternal and fetal heartbeat signals, measured in accordance with an embodiment of the present invention.

FIG. 12 is a graph showing body movement, in accordance with an embodiment of the present invention.

FIG. 13 is a graph showing restlessness events during normal sleep and during a clinical episode of asthma, in accordance with an embodiment of the present invention.

FIGS. 14A and 14B are graphs showing power spectrum densities of signals measured in accordance with an embodiment of the present invention.

FIG. 15 is a graph showing the result of the clinical score calculation as measured and analyzed in accordance with an embodiment of the present invention for an asthma patient.

FIG. 16 is a graph showing the correlation of heart rate and respiration rate in an asthma patient in accordance with an embodiment of the present invention.

FIG. 17 is an additional graph showing the correlation of heart rate and respiration rate in an asthma patient in accordance with an embodiment of the present invention.

FIG. 18 is a graph of several parameters measured for an asthma patient during a change in the treatment regimen of an asthma patient in accordance with an embodiment of the present invention.

FIG. 19 is a graph of the mechanical pressure signal during a night long measurement of an asthma patient and below that a graph of the standard deviation of that mechanical pressure signal in accordance with an embodiment of the present invention.

FIG. 20 is a graph of the mechanical pressure signal during an augmented breath, sigh or deep inspiration measured on an asthma patient in accordance with an embodiment of the present invention.

FIG. 21 is an additional graph of the mechanical pressure signal as measured during an augmented breath, sigh or deep inspiration measured on an asthma patient in accordance with an embodiment of the present invention.

FIG. 22 is a graph of the mechanical pressure signal of a measured on an asthma patient showing several respiration cycles in accordance with an embodiment of the present invention.

FIG. 23 is a graph of the multiple respiration cycles shown in FIG. 22 correlated by their peaks and shifted vertically, for display purposes only, in accordance with an embodiment of the present invention.

FIG. 24 is a graph of the average respiration cycle calculated by averaging the aligned cycles of FIG. 23 and showing an indication of the inspiration/expiration and rest sections in accordance with an embodiment of the present invention.

FIG. 25 is a graph of the average nightly respiration rates and heart rates for an asthma patient in accordance with an embodiment of the present invention.

FIG. 26 is a graph of multiple heart beat cycles as measured on an asthma patient with the peaks of the heart beat signal marked in accordance with an embodiment of the present invention.

FIG. 27 is a graph of the instantaneous heart rate signal of an asthma patient as calculated using the R-R method in accordance with an embodiment of the present invention.

FIG. 28 is a graph of the power spectrums of the signal of the same asthma patient for the same period of time as the graph in FIG. 27 showing the power spectrum of the filtered respiration signal, the power spectrum of the filtered heart signal, and the power spectrum of the heart rate signal shown in FIG. 27 in accordance with an embodiment of the present invention.

FIG. 29 is a graph illustrating data related to an event of central sleep apnea as measured and analyzed by an embodiment of the present invention.

FIG. 30 is a graph illustrating motion and acoustic data as measured and analyzed by an embodiment of the present invention.

FIG. 31 is a graph illustrating different acoustic signals as measured by an embodiment of the present invention.

FIG. 32 is a graph illustrating an acoustic signal of a cough comprising 3 phases as measured by an embodiment of the present invention.

FIG. 33 is a graph illustrating an acoustic signal of two coughs comprising 2 phases each as measured by an embodiment of the present invention.

FIG. 34 is a graph illustrating the behavior of AR time-frequency characteristic of an acoustic signal of a cough as measured and analyzed by an embodiment of the present invention.

FIG. 35 is a graph illustrating the signal envelope of the acoustic signal of a cough as measured and analyzed by an embodiment of the present invention.

FIG. 36 is a graph illustrating the acoustic signal of a vocal sound as measured and analyzed by an embodiment of the present invention.

FIG. 37 is a graph illustrating the distribution of frequencies of the acoustic signal of the vocal sound of FIG. 51 as measured and analyzed using a maximum/minimum analysis method by an embodiment of the present invention.

FIG. 38 is a graph illustrating the distribution of frequencies of the acoustic signal of the vocal sound of FIG. 51 as measured and analyzed using AR method by an embodiment of the present invention.

FIG. 39 is a graph illustrating the simultaneous acoustic signal and the mechanical motion signal of a cough event as measured by an embodiment of the present invention.

FIG. 40 is a graph illustrating the signal measured by an embodiment of the present invention with a chronic asthma patient during quiet sleep and in a restless period in sleep.

FIG. 41 is a graph illustrating the signal measured by an embodiment of the present invention with a chronic asthma patient and the threshold defined at different times during the night.

FIG. 42 is a graph illustrating the signal measured by an embodiment of the present invention monitoring a chronic asthma patient showing several posture changes during sleep.

FIG. 43 is a graph illustrating the signal measured by an embodiment of the present invention monitoring and the power spectrum of that signal.

FIG. 44 is a graph illustrating the signal measured by an embodiment of the present invention monitoring a human subject and the power spectrum of the demodulated signal.

FIG. 45 is a graph illustrating the signal measured by an embodiment of the present invention monitoring a human subject during an experiment of voluntarily induced increased tremor and the corresponding time dependent total spectrum power at the frequency band of 3-9 Hz.

FIG. 46 is a graph illustrating the output signal by an embodiment of the present invention monitoring a subject showing the breathing rate and breathing rate variability during sleep and indicating REM periods.

FIG. 47 is a graph illustrating the signal measured by an embodiment of the present invention monitoring a chronic asthma patient showing the respiration rate as measured during two different nights.

FIG. 48 is a graph illustrating the signal measured by an embodiment of the present invention monitoring a chronic asthma patient showing the ratio of respiration rate at the end of each night compared to the beginning of that night.

FIG. 49 is a graph illustrating the results of monitoring a chronic asthma patient by an embodiment of the present invention showing the results of PCA analysis of the nightly respiration rate patterns.

FIG. 50 is a graph illustrating the breathing related signal measured by an embodiment of the present invention monitoring a congestive heart failure patient showing a Cheyne Stokes Respiration pattern.

FIG. 51 is a graph illustrating the analysis of the respiratory pattern shown in FIG. 50 and analyzed by an embodiment of the present invention to show the time between consecutive respiratory cycles.

FIG. 52 is a graph illustrating the demodulated signal measured by an embodiment of the present invention monitoring a congestive heart failure patient with Periodic Breathing and the power spectrum of the demodulated signal calculated by an embodiment of the present invention.

FIG. 53 is a graph illustrating the breathing related signal measured by an embodiment of the present invention monitoring a congestive heart failure patient with the peak of each respiration cycle marked.

FIG. 54 is a graph illustrating the breathing cycle time as calculated by an embodiment of the present invention on a signal as shown in FIG. 53.

### DETAILED DESCRIPTION OF EMBODIMENTS

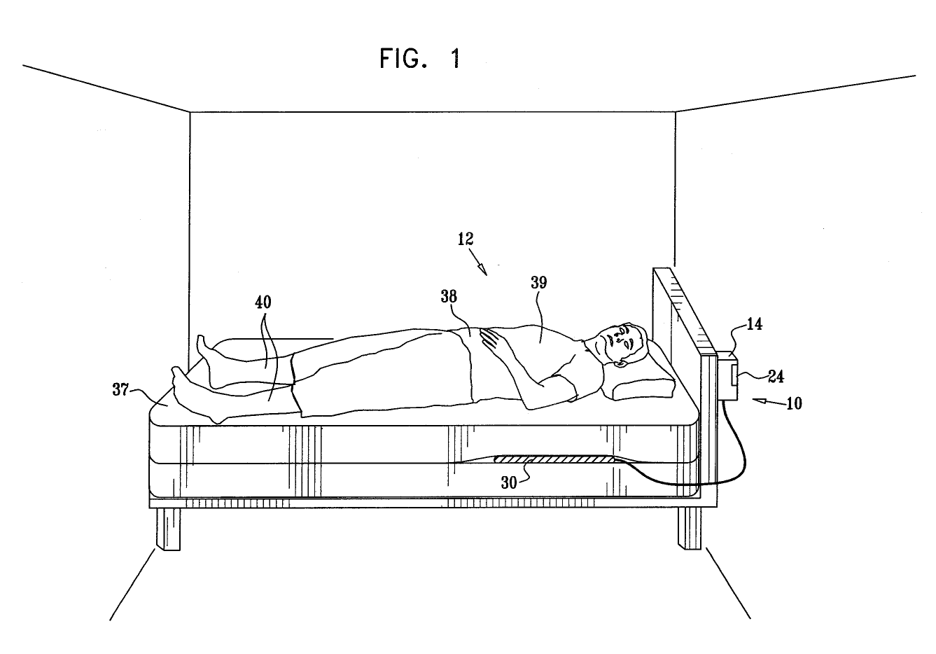

FIG. 1 is a schematic illustration of a system 10 for monitoring a chronic medical condition of a subject 12 in accordance with an embodiment of the present invention. System 10 typically comprises a motion sensor 30, a control unit 14, and a user interface (U/I) 24. For some applications, user interface 24 is integrated into control unit 14, as shown in the figure, while for other applications, the user interface and control unit are separate units. For some applications, motion sensor 30 is integrated into control unit 14, in which case user interface 24 is either also integrated into control unit 14 or remote from control unit 14.

As used herein, motion sensor 30 may be a “non-contact sensor,” that is, a sensor that does not contact the body or clothes of subject 12. Though in some aspects of the invention, sensor 30 may contact the body or clothes of subject 12, in many aspects, motion sensor 30 does not contact the body or clothes of subject 12. According to this aspect, by not contacting subject 12, sensor 30 may detect motion of patient 12 without discomforting patient 12. In some aspects, sensor 12 can perform its function without the knowledge of patient 12, for example, in special cases, without the consent of patient 12.

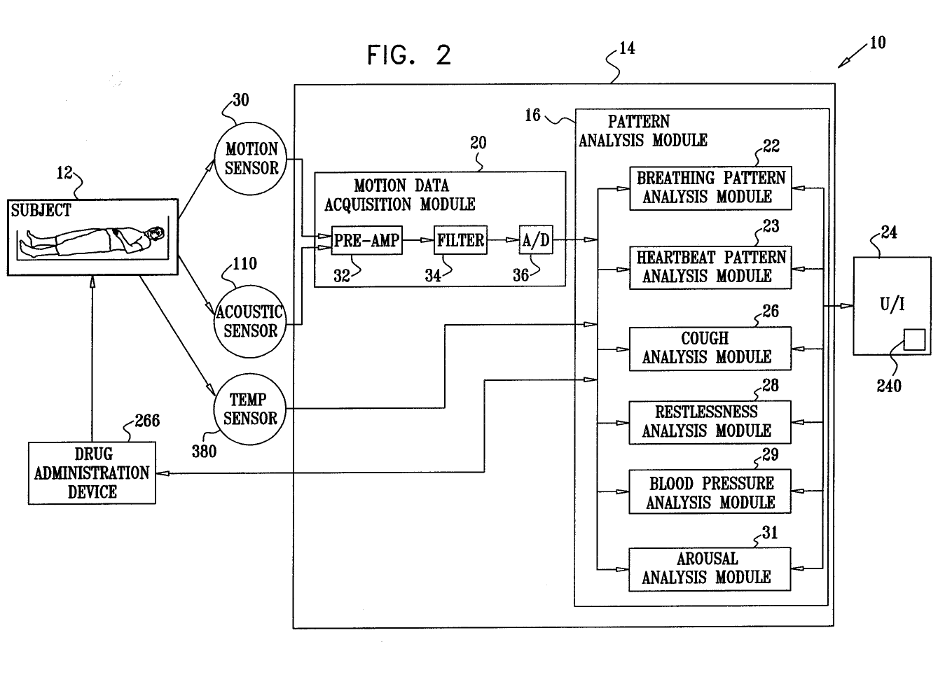

FIG. 2 is a schematic block diagram illustrating components of control unit 14 in accordance with an embodiment of the present invention. Control unit 14 typically comprises a motion data acquisition module 20 and a pattern analysis module 16. Pattern analysis module 16 typically comprises one or more of the following modules: a breathing pattern analysis module 22, a heartbeat pattern analysis module 23, a cough analysis module 26, a restlessness analysis module 28, a blood pressure analysis module 29, and an arousal analysis module 31. For some applications, two or more of analysis modules 20, 22, 23, 26, 28, 29, and 31 are packaged in a single housing. For other applications, the modules are packaged separately (for example, so as to enable remote analysis by one or more of the pattern analysis modules of breathing signals acquired locally by data acquisition module 20). For some applications, user interface 24 comprises a dedicated display unit such as an LCD or CRT monitor. Alternatively or additionally, user interface 24 includes a communication line for relaying the raw and/or processed data to a remote site for further analysis and/or interpretation.

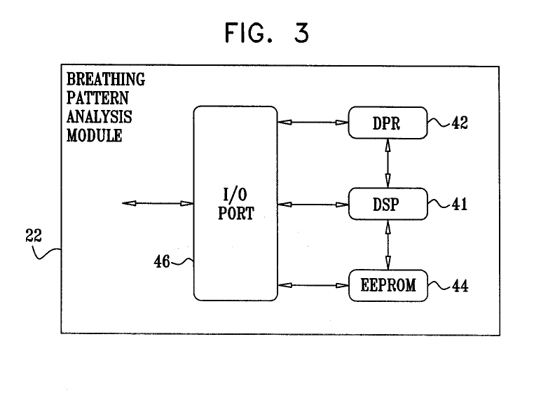

Breathing pattern analysis module 22 is adapted to extract breathing patterns from the motion data, as described herein below with reference to FIG. 3, and heartbeat pattern analysis module 23 is adapted to extract heartbeat patterns from the motion data. Alternatively or additionally, system 10 comprises another type of sensor, such as an acoustic sensor attached or directed at the subject's face, neck, chest, and/or back or placed under the mattress.

FIG. 3 is a schematic block diagram illustrating a breathing pattern analysis module 22 in accordance with an embodiment of the present invention. Breathing pattern analysis module 22 typically comprises a digital signal processor (DSP) 41, dual port RAM (DPR) 42, EEPROM 44, and an I/O port 46. Breathing pattern analysis module 22 is adapted to extract breathing patterns from the raw data generated by data acquisition module 20, and to perform processing and classification of the breathing patterns. Breathing pattern analysis module 22 analyzes changes in breathing patterns, typically during sleep. Responsively to the analysis, module 22 (a) predicts an approaching clinical episode, and/or (b) monitors episode severity and progression or shows or communicates other analysis results. Modules 23, 26, 28, 29, and 31 may be similar to module 22 shown in FIG. 3. For example, modules 23, 26, 28, 29, and 31 may include a digital signal processor, a dual port RAM, an EEPROM, and an I/O port similar to digital signal processor 41, dual port RAM 42, EEPROM 44, and an I/O port 46.

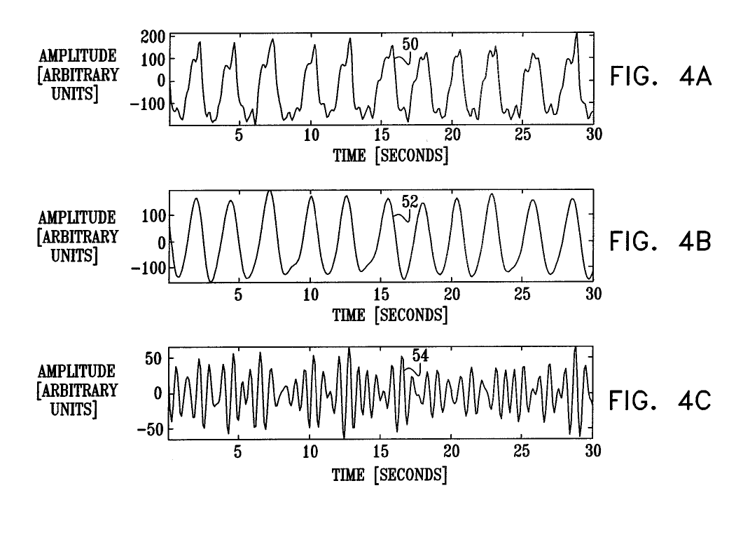

Reference is made to FIGS. 4A, 4B, and 4C which are graphs illustrating the analysis of motion signals measured in accordance with an embodiment of the present invention. Motion sensor 30 may comprise a vibration sensor, pressure sensor, or strain sensor, for example, a strain gauge, adapted to be installed under reclining surface 37, and to sense motion of subject 12. The motion of subject 12 sensed by sensor 30, for example, during sleep, may include regular breathing movement, heartbeat-related movement, and other, unrelated body movements, as discussed below, or combinations thereof. FIG. 4A shows raw mechanical signal 50 as measured by a piezoelectric sensor under a mattress, including the combined contributions of breathing- and heartbeat-related signals. Signal 50 was decomposed into a breathing-related component 52, shown in FIG. 4B, and a heartbeat-related component 54, shown in FIG. 4C, using techniques described herein below. All experimental results presented in the present application were measured using one or more piezoelectric sensors (nevertheless, the scope of the present invention includes performing measurements with other motion sensors 30, such as other pressure gauges or accelerometers.

In an embodiment of the present invention, data acquisition module 20 is adapted to non-invasively monitor breathing and heartbeat patterns of subject 12. Breathing pattern analysis module 22 and heartbeat pattern analysis module 23 are adapted to analyze the respective patterns in order to (a) predict an approaching clinical episode, such as an asthma attack or heart condition-related lung fluid buildup, and/or (b) monitor the severity and progression of a clinical episode as it occurs. User interface 24 is adapted to notify subject 12 and/or a healthcare worker of the predicted or occurring episode. Prediction of an approaching clinical episode facilitates early preventive treatment, which generally reduces the required dosage of medication, and/or lowers mortality and morbidity. When treating asthma, for example, such a reduced dosage generally minimizes the side-effects associated with high dosages typically required to reverse the inflammatory condition once the episode has begun.

Normal breathing patterns in sleep are likely to be subject to slow changes over days, weeks, months and years. Some changes are periodic due to periodic environmental changes like change in seasons, or to a periodic schedule such as a weekly schedule (for example outdoor play every Saturday), or biological cycles such as the menstrual cycle. Other changes might be monotonically progressive—for example, changes due to children growing up or adults aging. It is desirable to track these slow changes dynamically via an adaptive system.

In an embodiment of the present invention, system 10 is adapted to monitor parameters of the patient including breathing rate, heart rate, coughing counts, expiration/inspiration ratios, augmented breaths, deep inspirations, tremor, sleep cycle, and restlessness patterns, among other parameters. These parameters are defined herein as “clinical parameters.”

In an embodiment of the present invention, pattern analysis module 16 combines clinical parameter data generated from one or more of analysis modules 20, 22, 23, 26, 28, 29, and analyzes the data in order to predict and/or monitor a clinical event. For some applications, pattern analysis module 16 derives a score for each parameter based on the parameter's deviation from baseline values (either for the specific patient or based on population averages). Pattern analysis module 16 may combine the scores, such as by taking an average, maximum, standard deviation, or other function of the scores. The combined score is compared to one or more threshold values (which may be predetermined) to determine whether an episode is predicted, currently occurring, or neither predicted nor occurring, and/or to monitor the severity and progression of an occurring episode. For some applications, pattern analysis module 16 learns the criteria and/or functions for combining the individual parameter scores for the specific patient or patient group based on personal history. For example, pattern analysis module 16 may perform such learning by analyzing parameters measured prior to previous clinical events.

In one aspect, pattern analysis module 16 is adapted to analyze the respective patterns, for example, the patterns of slow changes mentioned above, in order to identify a change in baseline characteristic of the clinical parameters. For example, in order to identify the slow change in average respiration rate in sleep for a child due to growing up, a monthly average of the respiration rate in sleep is calculated. System 10 then calculates the rate of change in average respiration rate from one month to the next and displays that to the patient or healthcare professional. Additionally or alternatively, system 10 identifies that the average respiration rate in sleep during weekends is higher than on weekdays and uses in weekends a different baseline for comparison and decision on whether a clinical episodes is present or oncoming.

In one embodiment, system 10 monitors and logs the clinical condition of a patient over an extended period of time. During the same period of time, behavioral patterns, treatment practices and external parameters that may be affecting the patient's condition are monitored and logged as well. This information is input into system 10. System 10 calculates a score for the clinical condition of the patient based on the measured clinical parameters.

Although system 10 may monitor breathing and heartbeat patterns at any time, for some conditions it is generally most effective to monitor such patterns during sleep at night. When the subject is awake, physical and mental activities unrelated to the monitored condition often affect breathing and heartbeat patterns. Such unrelated activities generally have less influence during most night sleep. For some applications, system 10 monitors and records patterns throughout all or a large portion of a night. The resulting data set generally encompasses typical long-term respiratory and heartbeat patterns, and facilitates comprehensive analysis. Additionally, such a large data set enables rejection of segments contaminated with movement or other artifacts, while retaining sufficient data for a statistically significant analysis.

Reference is again made to FIG. 2. Data acquisition module 20 typically comprises circuitry for processing the raw motion signal generated by motion sensor 30, such as at least one pre-amplifier 32, at least one filter 34, and an analog-to-digital (A/D) converter 36. Filter 34 typically comprises a band-pass filter or a low-pass filter, serving as an anti-aliasing filter with a cut-off frequency of less than one half of the sampling rate. The low-passed data is typically digitized at a sampling rate of at least 10 Hz and stored in memory. For example, the anti-aliasing filter cut-off may be set to 10 Hz and the sampling rate set to 40 Hz. For some applications, filter 34 comprises a band-pass filter having a low cutoff frequency between about 0.03 Hz and about 0.2 Hz, e.g., about 0.05 Hz, and a high cutoff frequency between about 1 Hz and about 10 Hz, e.g., about 5 Hz. Alternatively or additionally, the output of motion sensor 30 is channeled through several signal-conditioning channels, each with its own gain and filtering settings tuned according to the desired signal. For example, for breathing signals, a relatively low gain and a frequency passband of up to about 5 Hz may be used, while for heartbeat signals, a moderate gain and a slightly higher frequency cutoff of about 10 Hz may be used. For some applications, motion sensor 30 is additionally used for registration of acoustic signals, for which a frequency passband of about 100 Hz to about 8 kHz is useful.

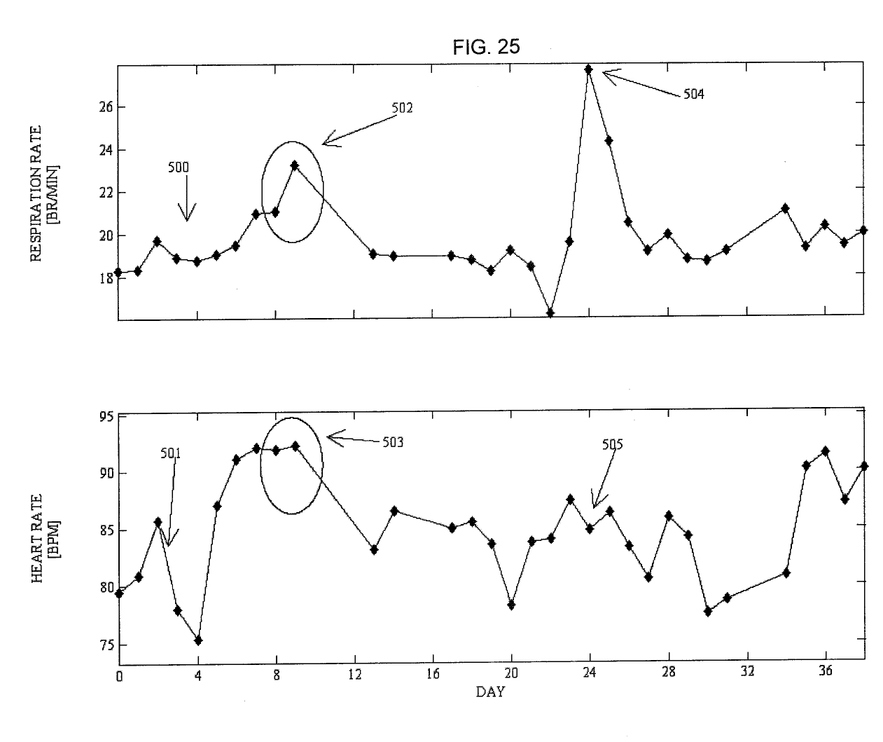

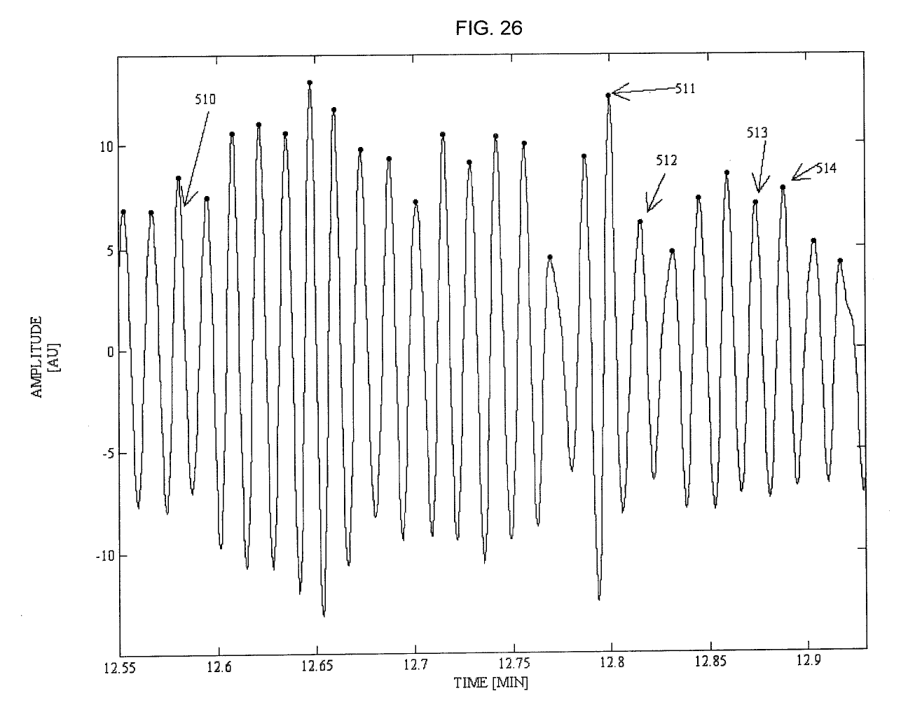

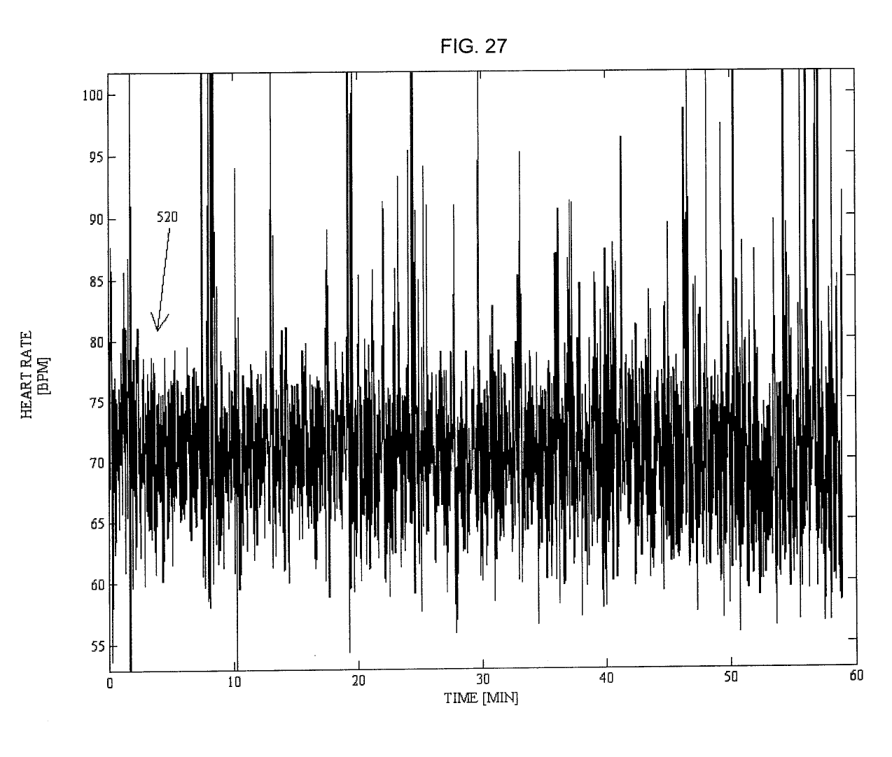

Chronic conditions often affect sleep cycles. For example, asthma affects the sleep cycle and the quality of sleep as described by Fitzpatrick and Engleman in Thorax, Vol. 46, pp. 569-573, which is incorporated herein by reference. In an embodiment of the present invention, system 10 is adapted to monitor heartbeat patterns of subject 12. The heart beat pattern is analyzed to identify peaks and measure distance between the peaks. FIG. 26 shows a typical signal measured by an embodiment of the present invention. Line 510 denotes the signal after a filter for the heartbeat signal (0.8-2.0 Hz). As is known in the art, the “R-R interval” is a characteristic of a heart beat signal, for example, an ECG trace. The R-R interval is the time period between successive R waves of the heart beat signal. According to aspects of the present invention, the R-R signal is calculated by measuring the time distance between each pair of peak, e.g., 511 to 512 and 513 to 514, and then dividing 60 seconds by that distance to receive the instantaneous heart rate in beats per minute (that is, 60 [secs./min.]/(R-R) [secs./beat]=60/(R-R) [beats/min.]). A sample result is shown in FIG. 27. This data is used to identify sleep stages using for example algorithms as described by Shinar et al. in Computers in Cardiology 2001; Vol. 28: 593-596 which is incorporated herein by reference.

Changes in length and periodicity of the different sleep stages are used as additional clinical parameters to identify an upcoming onset of a chronic condition, such as an asthma attack, congestive heart failure deterioration, cystic fibrosis related deterioration, diabetes hypoglycemia, epilepsy deterioration. In one embodiment, the above algorithm is used to identify the time and duration of deep sleep periods. In one embodiment, system 10 is used to identify the time, duration, and periodicity of REM sleep segments. This is then used as an additional clinical parameter for which a baseline is created and a change compared to baseline is identified and used to predict and monitor a clinical condition. For example, a change in the baseline periodicity of REM sleep for subject 12 may indicate the onset of an asthma attack or pulmonary edema.

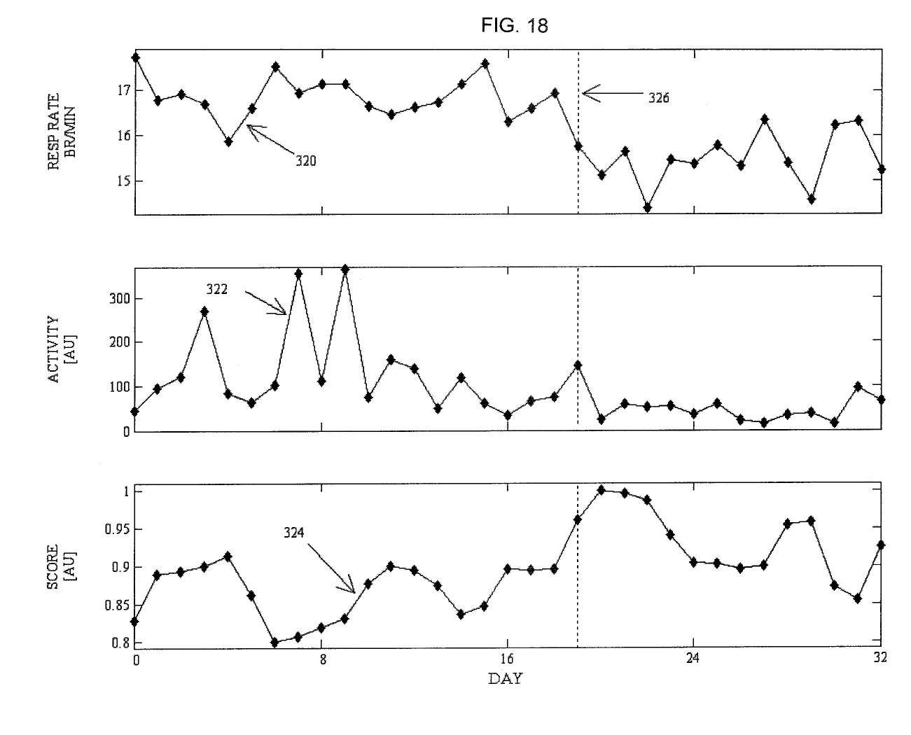

In an embodiment of the present invention, system 10 is adapted to monitor multiple clinical parameters such as respiration rate, heart rate, cough occurrence, body movement, deep inspirations, expiration/inspiration ratio, of subject 12. Pattern analysis module 16 is adapted to analyze the respective patterns in order to identify a change in the baseline pattern of the clinical parameters. In some cases, this change, whereas a new baseline is created significantly different from the previous baseline indicates, for example, a change in medication and provides the caregiver or healthcare professional with valuable feedback on the efficacy of treatment. FIG. 18, for example, shows actual results measured by an embodiment of the present invention on an asthma patient. Line 320 denotes the respiration rate average during sleep during the hours of 2:00 to 6:00 am for the patient. Line 322 denotes the activity level (restlessness) in sleep as calculated according to the present invention using the digital integration approach along the lines suggested by Ancoli-Israel S, Cole R, Alessi C et al. in the American Academy of Sleep Medicine Review Paper in SLEEP 2003;26(3):342-92 which is incorporated herein by reference. Line 324 denotes the asthma score calculated daily for the patient according to an embodiment of the present invention. Dotted line 326 denotes the date of a change in medication delivery device used by the monitored patient. In comparing the data calculated before and after the medication change, a statistically significant change in baseline was identified correlated with the medication change. A t-Test shows P<0.000001 for the average respiration rate, P<0.05 for the activity level, and P<0.004 for the Asthma score. The statistically significant changes show the physician that the change in medication is effective in improving the patient's clinical status.

In one embodiment, user interface 24 is adapted to notify subject 12 and/or a healthcare worker of the change in the baseline of the clinical parameters compared to the previous baseline, for example by performing t-Tests as described above. When treating a chronic condition, such an indication enables the patient or healthcare professional to optimize the dosage taken by the patient. For example, if the patient is taking medication which keeps him in good condition, the dosage may be decreased until a change in baseline compared to the starting baseline is identified. A dosage which is close to the minimum required to maintain the optimal baseline is then given to the patient. Such a reduced dosage generally minimizes the side-effects associated some of the asthma medications.

In one embodiment of the present invention, system 10 is adapted to monitor clinical parameters as defined herein above. Pattern analysis module 16 is adapted to analyze the respective patterns in order to identify changes due to medication and to provide feedback allowing optimization of the dosage of medication. For example, the medication given may be a type of beta-blocker. Beta-blockers are used to treat high blood pressure (hypertension), congestive heart failure (CHF), abnormal heart rhythms (arrhythmias), and chest pain (angina). Beta-blockers are sometimes used in Myocardial Infarction (MI) patients to prevent recurrence of MI. By measuring the heart rate patterns in sleep on a nightly basis, for example, the effect of the medication may be identified and the dosage increased or decreased until the optimal heart rate pattern is reached. The data is either reported to the patient or to the healthcare professional to adapt dosage or transmitted to an automatic dosage device which adapts the dosage accordingly.

In one embodiment, system 10 is used to identify the onset of unwanted side effects of medication, for example beta-blockers. The side effects include among others: wheezing, shortness of breath, slow heartbeat, and troubled sleep. These can be identified non-invasively by an embodiment of the present invention and the patient and/or caregiver is alerted.

Reference is again made to FIG. 1. In an embodiment of the present invention, motion sensor 30 comprises a pressure sensor (for example, a piezoelectric sensor) or an accelerometer, which is typically adapted to be installed in, on, or under a reclining surface 37 upon which the subject lies, e.g., sleeps, and to sense breathing- and heartbeat-related motion of the subject. Typically, reclining surface 37 comprises a mattress, a mattress covering, a sheet, a mattress pad, and/or a mattress cover. For some applications, motion sensor 30 is integrated into reclining surface 37, e.g., into a mattress, and the motion sensor and reclining surface are provided together as an integrated unit. For some applications, motion sensor 30 is adapted to be installed in, on, or under reclining surface 37 in a vicinity of an abdomen 38 or chest 39 of subject 12. Alternatively or additionally, motion sensor 30 is installed in, on, or under reclining surface 37 in a vicinity of a portion of subject 12 anatomically below a waist of the subject, such as in a vicinity of legs 40 of the subject. For some applications, such positioning provides a clearer pulse signal than positioning the sensor in a vicinity of abdomen 38 or chest 39 of the subject. For some applications, motion sensor 30 comprises a fiber optic sensor, for example as described by Butter and Hocker in Applied Optics 17: 2867-2869 (Sept. 15, 1978).

For some applications, pressure sensor (for example, the piezoelectric sensor) is encapsulated in a rigid compartment, which typically has a surface area of at least 10 cm², and a thickness of less than 5 mm. The sensor output is channeled to an electronic amplifier, such as a charge amplifier typically used with piezoelectric accelerometers and capacitive transducers to condition the extremely high output impedance of the transducer to a low impedance voltage suitable for transmission over long cables. The sensor and electronic amplifier translate the mechanical vibrations into electrical signals.

In an embodiment of the present invention, motion sensor 30 comprises a grid of multiple sensors, adapted to be installed in, on, or under reclining surface 37. The use of such a grid, rather than a single gauge, may improve breathing and heartbeat signal reception.

Breathing pattern analysis module 22 is adapted to extract breathing patterns from the motion data, as described herein below with reference to FIG. 3, and heartbeat pattern analysis module 23 is adapted to extract heartbeat patterns from the motion data. Alternatively or additionally, system 10 comprises another type of sensor, such as an acoustic or air-flow sensor, attached or directed at the subject's face, neck, chest, and/or back.

Reference is again made to FIG. 1. User interface 24 typically comprises a dedicated display unit, such as an LCD or CRT monitor. Alternatively or additionally, the output module comprises a wireless or wired communication port for relaying the acquired raw data and/or processed data to a remote site for further analysis, interpretation, expert review, and/or clinical follow-up. For example, the data may be transferred over a telephone line, and/or over the Internet or another wide-area network, either wirelessly or via wires.

In an embodiment of the present invention, motion data acquisition module 20 extracts breathing-related signals by performing spectral filtering in the range of about 0.05 to about 0.8 Hz, and heartbeat-related signals by performing spectral filtering in the range of about 0.8 to 5.0 Hz. For some applications, motion data acquisition module 20 adapts the spectral filtering based on the age of subject 12. For example, small children typically have higher breathing and heart rates, and therefore spectral filtering is typically set more tightly to the higher end of the frequency ranges, such as between about 0.1 and about 0.8 Hz for breathing, and between about 1.2 and about 5 Hz for heartbeat. For adults, spectral filtering is typically set more tightly to the lower end of the frequency ranges, such as between about 0.05 and about 0.5 Hz for breathing, and between about 0.5 and 2.5 Hz for heartbeat.

In some cases of non-invasive monitoring of clinical parameters, the quality of signal measured is dependent on patient size and weight, patient posture and location and mechanical characteristics of supporting devices such as bed mattresses. In some embodiments, a criterion is implemented for determining whether a specific measurement (e.g., during one minute) is of high quality and can be displayed to the patient or used in any follow on analysis. Such a criterion may be for example the amplitude of the measured signal, the amplitude of the relevant peak in the power spectrum of the measured signal, or other parameters. In mechanical measurements, the respiration signal is in most cases stronger and more clearly measured than the heart rate signal. In some body postures, in some embodiments, the heart rate related signal is so much smaller than the respiration signal that harmonics of the respiration signal may interfere with measurement of the heart rate. Therefore, in one embodiment, motion data acquisition module 20 extracts breathing-related signals by performing spectral filtering in the range of about 0.05 to about 0.8 Hz, and heartbeat-related signals by performing spectral filtering in the range of about 0.8 to 5.0 Hz. For each of the filtered signals a power spectrum is calculated and a largest peak is identified. The ratio of the heart rate related largest peak to the respiration related largest peak is calculated. This ratio is compared to a criterion which would typically be in the range of 0.02-0.25, for example 0.05. If the ratio is below that criterion, the heart rate measurement is disqualified and no measured value is provided for that time epoch. FIGS. 14A and 14B show the power spectrum of measured signal by an embodiment of the present invention. Peak 274 corresponds to the largest peak of the respiration signal and peak 276 corresponds to the largest peak of the heart rate signal. In FIG. 14A the ratio of the two peaks would be below the criterion and in FIG. 14B the ratio is above the criterion as set in that specific embodiment.

In an embodiment of the present invention, motion data acquisition module 20 extracts breathing-related signals by performing spectral filtering in the range of about 0.05 to about 0.8 Hz, and heartbeat-related signals by performing spectral filtering in the range of about 0.8 to 5.0 Hz. For each of the filtered signals a power spectrum is calculated and largest peak is identified. The amplitude of the peak corresponding to the second harmonic of the respiration rate is taken. The ratio of the heart rate related largest peak to the respiration related second harmonic peak is calculated. This ratio is compared to a criterion which would typically be in the range of 0.04-0.50, for example 0.10. If the ratio is below that criterion, the heart rate measurement is disqualified and no value is displayed or used for further analysis in that time segment.

In an embodiment of the present invention, motion data acquisition module 20 extracts breathing-related signals by performing spectral filtering in the range of about 0.05 to about 0.8 Hz, and heartbeat-related signals by performing spectral filtering in the range of about 0.8 to 5.0 Hz. For each of the filtered signals, a power spectrum is calculated and largest peak is identified. The ratio of the heart rate related peak to the respiration related peak is calculated. That ratio is plotted for the duration of the night. This ratio is generally expected to remain constant for as long at the subject is lying in the same position. For each two consecutive time epochs (an epoch typically being between 30-300 seconds, for example 60 seconds) the percentage of change of that ratio between the two epochs is calculated. Each time that ratio changes by more than a defined threshold (typically 10%-50%, for example 25%) system 10 considers it to be caused by a change in body posture. The frequency and timing of these changes is measured as an indication for restlessness in sleep.

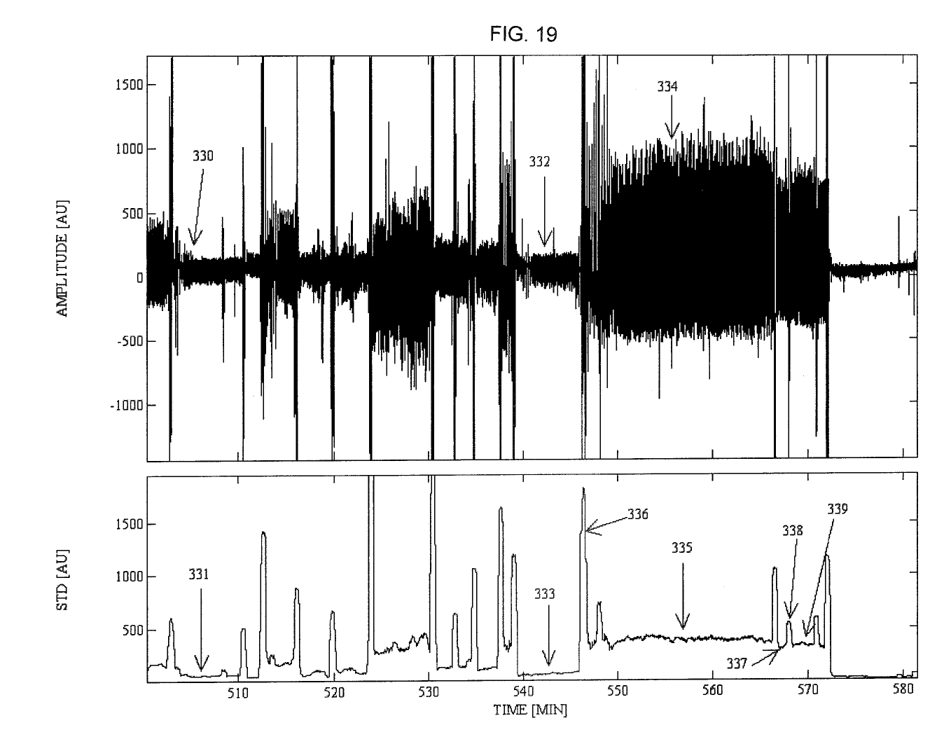

In an embodiment of the present invention the standard deviation (STD) of the measured signal is calculated for each time epoch, for example, one minute. The STD of the signal during consecutive minutes is expected to be quite similar during sleep unless the subject changes sleeping positions. A criterion for the extent of change in STD between consecutive minutes is defined, typically 10%-50%, for example, 25%. Each time a change of larger magnitude than the criterion is identified, an event is defined and counted. The total number of such events and their distribution during the sleeping period is logged as an indication of body position change. In one embodiment, such an event is logged only if a change in STD is identified simultaneously with a restlessness event. FIG. 19 shows the mechanical signal as measured by an embodiment of the present invention and the STD for each time epoch in that measurement. Line 330 shows the mechanical pressure signal as measured; area 332 has an STD that is shown in area 333; area 334 has an STD which is shown in area 335. The STD level shown in 335 is significantly higher than shown in 333. Between 335 and 333 is an area of significant restlessness marked as 336. System 10 therefore identifies event 336 as a change in body posture. On the other hand, 337 and 339 show a similar level of STD. Therefore system 10 does not identify event 338 as a change in body posture. The number and distribution of body posture changes during sleep is an indication to the level of restlessness in sleep which is a clinical parameter used to identify clinical conditions.

In an embodiment of the present invention, system 10 is used in conjunction with a Nitric Oxide monitor such as developed by Aperon Biosystems Corp. of Menlo Park, Calif., USA and Aerocrine AB of Solna, Sweden. The data measured by the Nitric Oxide meter is communicated into pattern analysis module 16 and used as an additional clinical parameter in conjunction with other clinical parameters measured by system 10 in order to identify the onset of a clinical episode, for example an asthma episode.

In an embodiment of the present invention, the acoustic sensor 110 is implemented with a membrane such as that usually present in a stethoscope in order to efficiently sense the audio signal. This membrane can be placed under a mattress, mattress pad or mattress cover.

In an embodiment of the present invention, system 10 is used to identify the onset of epilepsy seizures by a characteristic change in the pattern of respiration, heart rate, and tremor. The result of the analysis by system 10 is used to determine the timing of Vagus Nerve Stimulation (VNS). VNS is designed to prevent seizures by sending regular, mild pulses of electrical energy to the brain via the vagus nerve. These pulses are supplied by a device similar to a pacemaker, for example the VNS devices developed by Cyberonics of Houston, Tex.

Patients suffering from asthma often reach the Emergency Room. Upon presenting at the Emergency Room, they are sometimes erroneously diagnosed to be suffering from anxiety attack. This has been known to lead to clinical deterioration and may even cause death. In one embodiment, system 10 differentiates between anxiety attacks and asthma attacks. During sleep, anxiety is to a large extent habituated and thus does not present the same respiration patterns as measured in an asthma attack. Thus, system 10 verifies that subject 12 is suffering from an asthma attack and not an anxiety attack if it identified during sleep the characteristic respiration pattern changes described herein. This information is communicated to the patient, care taker, physician, or any other entity that may make clinical determination regarding the patient.

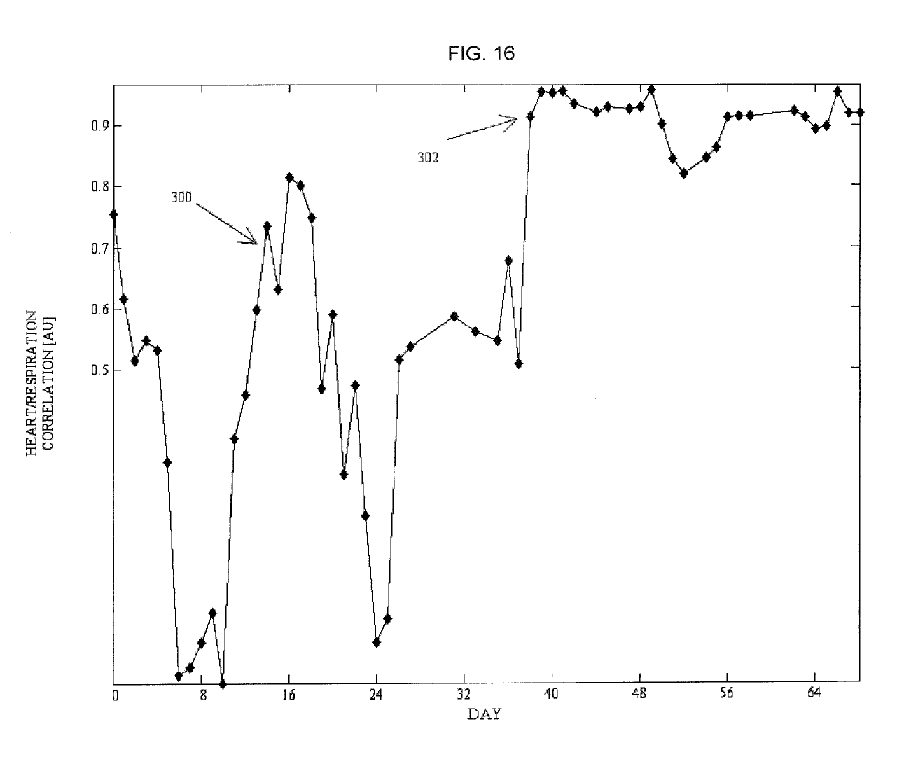

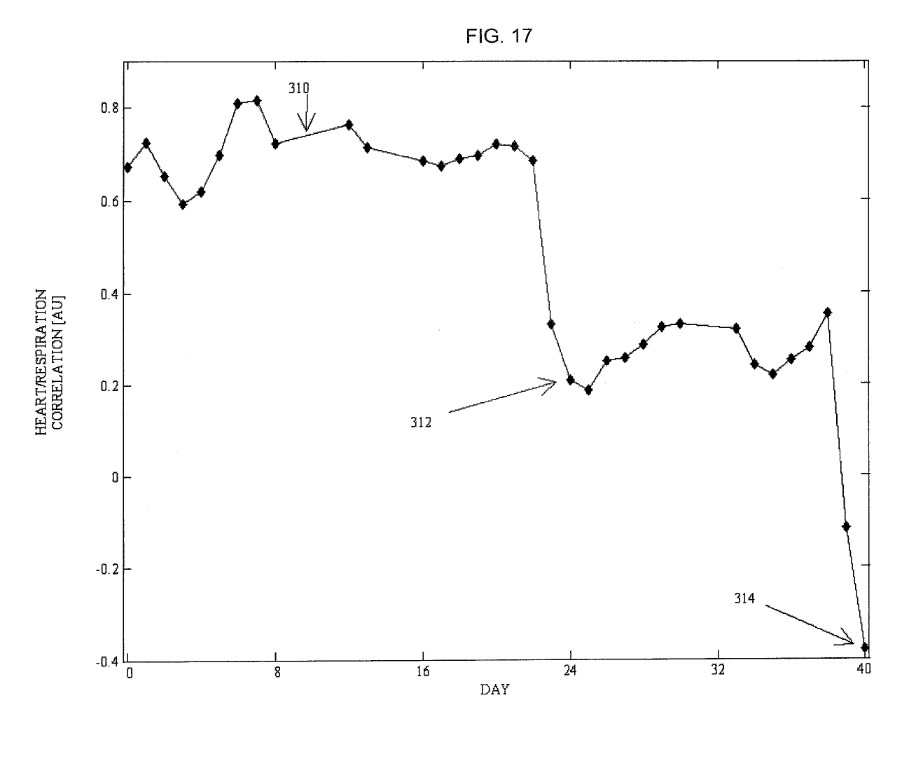

In one embodiment, system 10 calculates the average respiration rate and heart rate for predefined time segments. Such time segments can be minutes, hours, or days. By analyzing the history of the patient the system can calculate the correlation of respiration rate and heart rate patterns. When an onset of an asthma attack approaches the correlation of heart rate and respiration rate pattern shows a clear change. For each night the respiration rate and heart rate in sleep during the hours of 11:00 pm to 6:00 am is averaged. For each date, a respiration vector of length N with the average respiration rate of the last N nights and a heart rate vector of length N with the average heart rate for the last N nights is defined. N is typically between 3 and 30, for example 10. The correlation coefficient of the heart rate vector and the respiration vector is calculated for each date by system 10. A moving window of several days is used to calculate correlation coefficient changes between the respiration and heart rate vectors. A steady correlation coefficient pattern over at least several days is required to identify a significant change of correlation coefficient from one time interval to another. A significant change is defined as a change in the correlation coefficient level of a magnitude larger than the typical correlation coefficient variation in the previous time interval, e.g., a change larger than 3 standard deviations of the correlation coefficient signal in the previous time interval. System 10 identifies such a significant change as an indication for an eminent clinical episode. FIG. 16 and FIG. 17 show the correlation coefficient results for two different asthma patients. Lines 300 and 310 show the correlation coefficient calculated between the heart rate vector and respiration vector with N=10 in an embodiment of the present invention. Points 302, 312, and 314 represent dates of asthma exacerbations and clearly a significant change in correlation coefficient level is seen on or before those dates.

In one embodiment, system 10 measures respiration rate, heart rate during sleep and identifies restlessness events. The correlation of changes in respiration rate and heart rate patterns with the occurrence of restlessness events is used as an indicator for the onset of a clinical episode such as an asthma exacerbation, COPD deterioration or CHF deterioration. For example, an increased correlation between restlessness event timing and increases in heart and respiration rates are a positive indicator for an asthma exacerbation.

Premature babies, preemies, often need to be closely monitored at home or at the hospital to provide early warning of deterioration of condition due to infection, for example. In one embodiment, system 10 is used to closely monitor preemies in a contact-less manner and provide a warning to a parent or healthcare professional upon any change in clinical parameters measured.

In one embodiment, system 10 is used to monitor chronic patients of asthma. System 10 differentiates between an event of fever and an event of asthma deterioration by identifying different clinical parameters for each. FIG. 25 shows the respiration rate and heart rate pattern for an asthma patient monitored with an embodiment of the present invention. Each data point represents the average during the hours of 11:00 pm-6:00 am of the respiration rate and heart rate during sleep. The days marked as 502 and 503 are identified by the system as fever events and the day marked as 504 and 505 is identified as an asthma event. The differentiation by system 10 is done as follows: in 502 and 503 the relative increase in heart rate is much higher than in respiration rate and the increase in heart rate occurs before the increase in respiration rate. On the other hand, in an asthma event, the respiration rate has an earlier and much more significant increase than the heart rate.

In one embodiment, system 10 measures the clinical parameters of subject 12 while in bed, for example with a contact-less sensor. In order to analyze variation compared to baseline in the clinical parameters, system 10 discards any data in which the patient was awake and uses only measurements while the subject was asleep. Identification of sleep is done using the R-R methods described herein above or the periodicity of the respiration pattern.

In one embodiment, system 10 discards any data while subject 12 showed significant restlessness. Thus for example, the first few minutes the patient is in bed and is still tossing and turning, with his large body movements having significantly stronger signals than the cyclic respiration pattern, are discarded from this analysis.

In one embodiment, during sleep, sleep stage is identified using techniques described herein above. For each identified sleep stage, the average respiration rate, heart rate and other clinical parameters are calculated. This data is compared to baseline defined for that subject for each identified sleep stage, in order to identify the onset or progress of a clinical episode.

In one embodiment, for each night, for each hour of sleep, counted from the onset of sleep, the average respiration rate, heart rate and other clinical parameters are calculated. This data is compared to baseline in order to identify the onset or progress of a clinical episode.

In one embodiment, for each night, for each hour, the average respiration rate, heart rate and other clinical parameters are calculated. This data is compared to baseline in order to identify the onset or progress of a clinical episode. For example, the average respiration rate in sleep during 2:00 AM-3:00 AM is calculated and compared to baseline for that subject in order to identify the onset or progress of a clinical episode.

In one embodiment, system 10 identifies a trend of change of one or more of the clinical parameters measured as an indication in order to identify the onset or progress of a clinical episode. For example, when system 10 identifies a consecutive increase in respiration rate over 3 nights, it indicates that an asthma exacerbation is likely.

In one embodiment, system 10 monitors and logs the clinical condition of a patient over an extended period of time. During the same period of time, behavioral patterns, treatment practices and external parameters that may be affecting the patient's condition are monitored and logged as well. This information is input into system 10. System 10 calculates a score for the clinical condition of the patient based on the measured clinical parameters. System 10 calculates the correlation coefficient of that clinical score with behavioral, treatment and external patterns. Positive correlation between the score and a pattern indicates to the patient or physician a possible causal connection between that parameter and the patient's clinical condition. For example, System 10 correlates the changes in the clinical condition of an asthma patient with the several parameters: weather, outdoor play, use of beta agonists and cleaning of the home or other interventions by asthma support groups such as Healthy Home Resources of Pittsburgh, Pa. For example, system 10 then identifies that each time the house is cleaned from dust mites by representatives of Healthy Home Resources, the asthma score of the patient shows an improvement by 5%. That information is presented to the patient, caregiver, or healthcare professional in order to adapt the lifestyle of the patient for optimal quality of life.

In one embodiment, multiple systems 10 are used to monitor patients in a living or working in proximity, for example in inner city blocks or in a large workplace, the clinical condition of each patients is monitored by a system 10. The clinical scores of the patients are correlated with each other and with behavioral, external, and clinical parameters to evaluate the possible general impact of such parameters. Positive correlation between clinical scores of multiple subjects with external, clinical or behavioral parameters is a strong indication for the causal relation between the parameter and the clinical condition of the subjects. This can be valuable for large employers that have groups of employees working in situations that can risk their health condition.

In one embodiment, the system calculates an asthma score based on the different parameters. For example, the formula for the asthma score may be:

$$S ( D ) = \frac{\begin{matrix}20 {R}_{a} ( D ) + 20 {R}^{′} ( D ) + 20 {R}_{b} ( D ) + 10 {HR}_{a} ( D ) + \\ 10 {HR}^{′} ( D ) + AC ( D ) + 5 SE ( D ) + 5 DI ( D )\end{matrix}}{N}$$

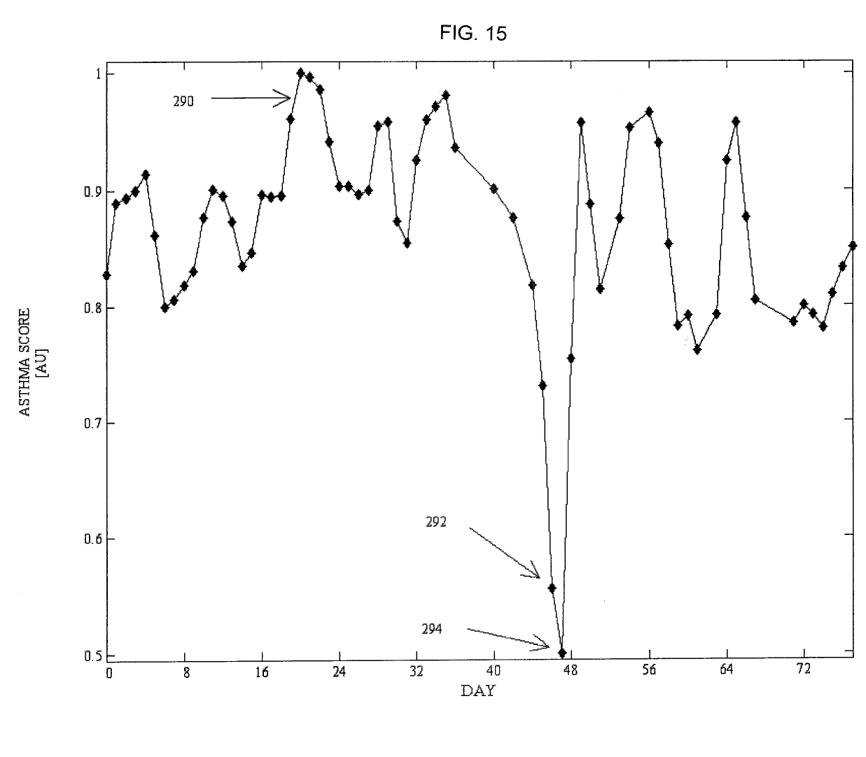

S(D)—Score for Date D
Rₐ(D)—Average respiration rate divided by the average respiration rate for all previous measured nights.
R′(D)—First derivative of the respiration rate calculated as follows:

$${R}^{′} ( D ) = \frac{R ( D ) - R ( D - 1 )}{R ( D - 1 )}$$
where R (D) is the average respiration rate of the subject for day D and R(D-1) is the average respiration rate of the subject for the day prior to day D;
R_{b}(D)—Average respiration rate for the night prior to date D divided by the average respiration rate over the previous 3 nights.
HRₐ(D)—Average heart rate divided by the average heart rate for all previous measured nights.
HR′(D)—First derivative of the average heart rate calculated as follows:

$${HR}^{′} ( D ) = \frac{HR ( D ) - HR ( D - 1 )}{HR ( D - 1 )}$$
where HR(D) is the average heart rate of the subject for day D and HR(D-1) is the average heart rate of the subject for the day prior to day D;
AC(D)—is the measure of activity level during sleep (restlessness) divided by the average of that measure for all previously measured nights.
SE(D)—Sleep efficiency as for that night divided by the average sleep efficiency for all previously measured nights
DI(D)—Deep Inspirations for that night divided by the average number of deep inspirations for all previously measured nights
N—is an integer dependent upon the illness under consideration, among other things, and may have a value between 80 and 110, typically, 88 to 92, for example, about 91.

Where each of the above parameters is calculated for the duration of the sleep time or specific hours during the night prior to date D. FIG. 15 shows an example of a similarly calculated asthma score, for a value of N of 91, but inverted to make the higher score indicate better clinical condition and normalized between 1.0 and 0.5. Line 290 is a graph of such a score calculated for an asthma patient. The day denoted by arrow 294 represents a date of an asthma exacerbation.

The values of Rₐ(D), HRₐ(D), AC(D), SE(D), and DI(D) may be calculated for at least three days prior to day D, for example, for at least three successive days immediately prior to day D. Alternatively, Rₐ(D), HRₐ(D), AC(D), SE(D), and DI(D) may be calculated as a ratio of that date's parameter and the average over K nights where K would typically be in the range of 7 to 365, for example, K may be 30. K may also be successive nights, for example, K successive nights before day D. Alternatively, Rₐ(D), HRₐ(D), AC(D), SE(D), and DI(D) can be calculated as a ratio of that date's parameter and the average over the past K nights that have not included an exacerbation of the chronic condition. This exacerbation being identified either manually through user input or automatically by system 10. In one embodiment, the average heart rate for each minute of sleep is calculated and then the standard deviation of that time series is calculated. This standard deviation is added as an additional parameter to, for example, a score equation similar to the above asthma score equation for the patient.

In one embodiment, system 10 is used to monitor the patients' long-term status and identify any clinical change caused by an alteration in the patients' therapeutic regime. For example, Pfizer Inc. of New York, N.Y. is in final regulatory approval stages of an inhaled insulin treatment called Exubera for diabetic patients. However, there are concerns that the inhaled drug may affect respiratory function. In one embodiment, system 10 is used to monitor respiratory and heart function in a contact-less manner before and after the use of Exubera by a patient to identify whether there is any affect on respiratory function by monitoring changes in clinical parameters. This enables early identification of side effects such as respiration related side effects of the drug and therefore enable wider use of the drug even for patients who may be considered at higher risk of respiratory system damage such as asthma and COPD patients.

In one embodiment, system 10 includes a motion sensor 30 that is implemented on top of a mattress. For example the sensor is implemented in a pillow or a “teddy bear” and so becomes easily movable from one bed to another and easy to travel with for children and adults.

In one embodiment, sensor 30 senses frequencies higher than respiration and heart rate yet lower than the acoustic range for example in the range of 3 Hz to 20 Hz. These frequencies are used to identify tremor and coughs.

In one embodiment system 10 calculates a disease related score over a period of several days. The variability of that score over a time period of several days, for example two weeks, is measured and presented to the patient and/or healthcare professional as an estimate of the stability of the disease status of the patient.

In one embodiment, system 10 measures the status of a chronic patient while he is on his regular set of medication, then for a limited period of time a higher dose or stronger medication is given in order to measure a reference “optimal” baseline that is achieved when the patient is under the stronger medication. This optimal baseline is then used as reference in order to identify whether the patient is held close to his optimal performance with the regular set of medication. If not, the healthcare professional may decide to change the medication and/or offer additional treatment. For example, if for an asthma patient, a week long course of oral steroids is shown to reduce the average nightly respiration rate by more than 3 breaths per minute then the healthcare professional may decide that the current standard medication is not strong enough and a different long term medication is required. Or, an asthma patient that is not taking any anti-inflammatory medication, may be given a 2 week course of inhaled corticostereoids, if a significant improvement in respiration pattern is identified (i.e. reduction in average respiration rate and/or significant change in expiration/inspiration ratio, or a significant reduction in score variability, etc.) then the healthcare professional may decide to prescribe the patient daily use of this medication.

In one embodiment, system 10 is used to collect patient clinical parameters and build a personal database for the patient. Over an extended time period of months and years this database can provide the patient and healthcare professional a valuable perspective on long term/slow trend processes taking place. This can be used to compare patient trends to population averages to help diagnose conditions and to assist in treatment decision making. For example, long term data on sleep respiration rates is used to draw a graph showing respiration rate versus age curve. For children, respiration rate is expected to decrease as age increases. For some asthma patients, the respiration rate does not decrease with age. This can help diagnose asthma or assist in treatment decision. This serves as a prognosis tool showing whether the patient's condition is improving (curve gradually getting closer to population average) or deteriorating (curve showing gradual increase in difference from population average). The logged parameters are not limited to the respiration rate, all the parameters previously mentioned can be logged by this system. In young children, system 10 may be used to log such data compared to population average and identify patients whose parameter pattern indicate potential for asthma. Early identification and early treatment allows more effective prevention of severe exacerbations reducing treatment costs and patient suffering.

For some applications, motion data acquisition module 20 extracts breathing rate and heart rate from the filtered signal using zero-crossings or power spectrum analyses.

As mentioned above, motion of the subject during sleep includes regular breathing-related and heartbeat-related movements as well as other, unrelated body movements. In general, breathing-related motion is the dominant contributor to body motion during sleep. Pattern analysis module 16 is adapted to substantially eliminate the portion of the motion signal received from motion data acquisition module 20 that represents motion unrelated to breathing and heartbeat. For example, the pattern analysis module may remove segments of the signal contaminated by non-breathing- and non-heartbeat-related motion. While breathing- and heartbeat-related motion is periodic, other motion is generally random and non-predictable. For some applications, the pattern analysis module eliminates the non-breathing- and non-heartbeat-related motion using frequency-domain spectral analysis or time-domain regression analysis. Techniques for applying these analysis techniques will be evident to those skilled in art who have read the present application. For some applications, pattern analysis module 16 uses statistical methods, such as linear prediction or outlier analysis, to remove non-breathing-related and non-heartbeat-related motion from the signal. Motion data acquisition module 20 typically digitizes the motion data at a sampling rate of at least 10 Hz, although lower frequencies are suitable for some applications.

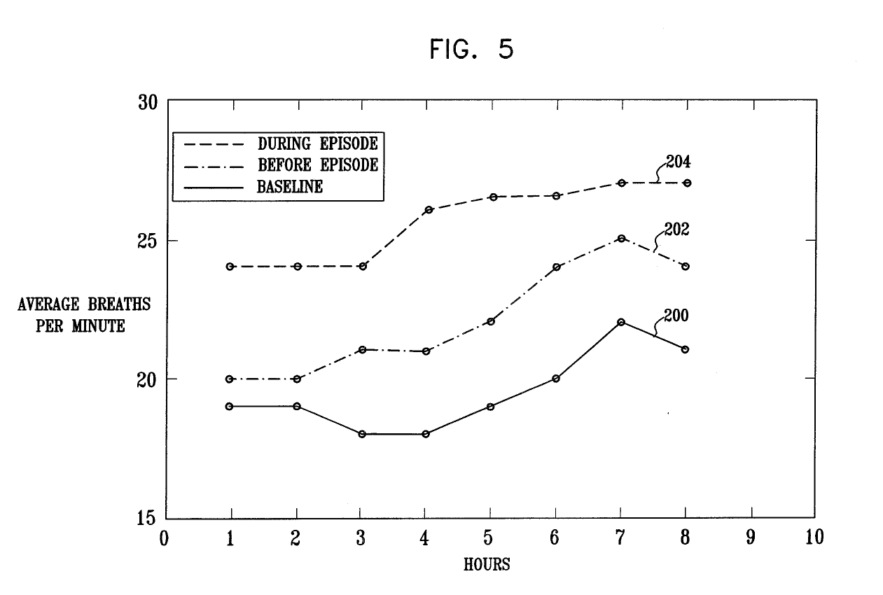

Breathing pattern analysis module 22 is typically adapted to extract breathing patterns from a train of transient breathing pulses, each pulse including one inhalation-exhalation cycle. Breathing patterns during night sleep generally fall into one of several categories, including:
relatively fast-changing, random breathing patterns, which occur mainly during REM sleep;
cyclic breathing rate variability patterns, whose typical duration ranges from several seconds to several minutes, e.g. Cheyne-Stokes Respiration (CSR) or periodic breathing;
slow trends in breathing rates (typically, during normal sleep of a healthy subject, such slow trends include segmented, substantially monotonically declining breathing rates usually lasting several hours; for subjects suffering chronically from certain conditions, such as asthma, the monotonic decline may be less pronounced or absent, as discussed, for example, herein below with reference to FIG. 5);
interruptions in breathing patterns such as coughing and other sleep disturbances; and
interruptions in breathing patterns caused by momentary waking.

These breathing patterns are associated with various physiological parameters, such as sleep-stage, anxiety, and body temperature. For example, REM sleep is usually accompanied by randomly variable breathing patterns, while deep sleep stages are usually accompanied by more regular and stable patterns. Abnormally high body temperature may accelerate breathing rate, but usually maintains normal cyclic breathing rate variability patterns. Psychological variables such as anxiety are also modulators of breathing patterns during sleep, yet their effect is normally reduced with sleep progression. Interruptions in breathing patterns such as coughing or that caused by momentary waking may be normal, associated with asthma, or associated with other unrelated pathology, and are assessed in context.

In an embodiment of the present invention, pattern analysis module 16 is configured to predict the onset of an asthma attack, and/or monitor its severity and progression. Pattern analysis modules 22 and 23 typically analyze changes in breathing rate patterns, breathing rate variability patterns, heart rate patterns, and/or heart rate variability patterns in combination to predict the onset of an asthma attack. For some applications, breathing and/or heart rates are extracted from the signal by computing the Fourier transform of the filtered signal, and finding the frequency corresponding to the highest spectral peak value within allowed ranges corresponding to breathing and heart rate, or by using a zero-crossing method, or by finding the peaks of the time-domain signal and averaging the inter-pulse time over one minute to find heart beats per minute. For some applications, such averaging is performed after removing outlying values.

Although breathing rate typically slightly increases prior to the onset of an attack, this increase alone is not always a specific marker of the onset of an attack. Therefore, in order to more accurately predict the onset of an attack, and monitor the severity and progression of an attack, in an embodiment of the present invention, breathing pattern analysis module 22 additionally analyzes changes in breathing rate variability patterns. For some applications, module 22 compares one or more of the following patterns to respective baseline patterns, and interprets a deviation from baseline as indicative of (a) the onset of an attack, and/or (b) the severity of an attack in progress:
a slow trend breathing rate pattern. Module 22 interprets as indicative of an approaching or progressing attack an increase vs. baseline, for example, for generally healthy subjects, an attenuation of the typical segmented, monotonic decline of breathing rate typically over at least 1 hour, e.g., over at least 2, 3, or 4 hours, or the transformation of this decline into an increasing breathing rate pattern, depending on the severity of the attack;
a breathing rate pattern. Module 22 interprets as indicative of an approaching or progressing attack an increase or lack of decrease in breathing rate during the first several hours of sleep, e.g., during the first 2, 3, or 4 hours of sleep.
a breathing rate variability pattern. Module 22 interprets as indicative of an approaching or progressing attack a decrease in breathing rate variability. Such a decrease generally occurs as the onset of an episode approaches, and intensifies with the progression of shortness of breath during an attack;
a breathing duty-cycle pattern. Module 22 interprets a substantial increase in the breathing duty-cycle as indicative of an approaching or progressing attack. Breathing duty-cycle patterns include, but are not limited to, inspirium time/total breath cycle time, expirium time/total breath cycle time, and (inspirium+expirium time)/total breath cycle time;
a change in breathing rate pattern towards the end of night sleep (typically between about 3:00 A.M. and about 6:00 A.M.); and
interruptions in breathing pattern such as caused by coughs, sleep disturbances, or waking. Module 22 quantifies these events, and determines their relevance to prediction of potential asthma attacks.

Pattern analysis modules 22 and 23 typically determine baseline patterns by analyzing breathing and/or heart rate patterns, respectively, of the subject during non-symptomatic nights. Alternatively or additionally, modules 22 and 23 are programmed with baseline patterns based on population averages. For some applications, such population averages are segmented by characteristic traits such as age, height, weight, and gender.

In an embodiment of the present invention, pattern analysis module 16 determines the onset of an attack, and/or the severity of an attack in progress, by comparing the measured breathing rate pattern to a baseline breathing rate pattern, and/or the measured heart rate pattern to a baseline heart rate pattern.

In an embodiment of the present invention, breathing pattern analysis module 22 passes the respiration rate pattern calculated for the subject's sleep time through a low pass filter (e.g., a Finite Impulse Response filter) to reduce short-term effects such as REM sleep. For some applications, heartbeat pattern analysis module 23 performs similar filtering on the heart rate data.

Reference is made to FIG. 5, which is a graph illustrating breathing rate patterns of a chronic asthma patient, measured during an experiment conducted in accordance with an embodiment of the present invention. Breathing of the asthma patient was monitored during sleep on several nights. The patient's breathing rate was averaged for each hour of sleep (excluding periods of rapid eye movement (REM) sleep, which were removed using a low pass filter, which reduces the short-term effect of REM sleep; alternatively, REM sleep is identified and removed from consideration). During the first approximately two months that the patient was monitored, the patient did not experience any episodes of asthma. A line 200 is representative of a typical slow trend breathing pattern recorded during this non-episodic period, and thus represents a baseline slow trend breathing rate pattern for this patient. It should be noted that, unlike the monotonic decline in breathing rate typically observed in non-asthmatic patients, the baseline breathing rate pattern of the chronically asthmatic patient of the experiment reflects an initial decline in breathing rate during the first few hours of sleep, followed by a gradual increase in breathing rate throughout most of the rest of the night.

Lines 202 and 204 were recorded on two successive nights at the conclusion of the approximately two-month period, line 202 on the first of these two nights, and line 204 on the second of these two nights. The patient experienced an episode of asthma during the second of these nights. Lines 202 and 204 thus represent a pre-episodic slow trend breathing rate pattern and an episodic slow trend breathing rate pattern, respectively. As can be seen in the graph, the patient's breathing rate was elevated by about 1-3 breaths per minute vs. baseline during all hours of the pre-episodic night, and was even further elevated vs. baseline during the episodic night.

Using techniques described herein, breathing pattern analysis module 22 compares the pattern of line 202 with the baseline pattern of line 200, in order to predict that the patient may experience an asthmatic episode. Module 22 compares the pattern of line 204 with the baseline pattern of line 200 in order to assess a progression of the asthmatic episode.

In an embodiment of the present invention, the deviation from baseline is defined as the cumulative deviation of the measured pattern from the baseline pattern. A threshold indicative of a clinical condition is set equal to a certain number of standard errors (e.g., one standard error). Alternatively or additionally, other measures of deviation between measured and baseline patterns are used, such as correlation coefficient, mean square error, maximal difference between the patterns, and the area between the patterns. Further alternatively or additionally, pattern analysis module 16 uses a weighted analysis emphasizing specific regions along the patterns, for example, by giving a double weight to the first two hours of sleep or the hours of 3:00-6:00 a.m.

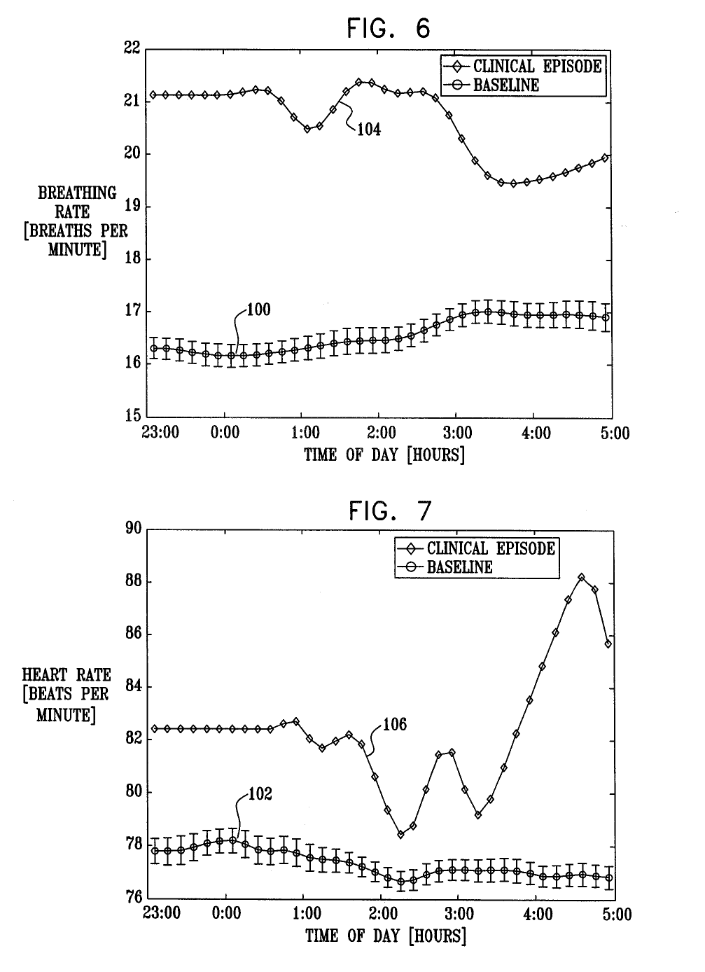

FIGS. 6 and 7 are graphs of exemplary baseline and measured breathing rate and heart rate nighttime patterns, respectively, measured in accordance with an embodiment of the present invention. Lines 100 and 102 (FIGS. 6 and 7, respectively) represent normal baseline patterns in the absence of an asthma attack. The bars represent one standard error. Lines 104 and 106 (FIGS. 6 and 7, respectively) represent patterns during nights prior to an onset of an asthma attack. Detection of the change in pattern between lines 100 and 102 and lines 104 and 106, respectively, enables the early prediction of the approaching asthma attack.

In an embodiment of the present invention, pattern analysis module 16 is configured to predict the onset of a clinical manifestation of heart failure, and/or monitor its severity and progression. Module 16 typically determines that an episode is imminent when the module detects increased breathing rate accompanied by increased heart rate, and/or when the monitored breathing and/or heartbeat patterns have specific characteristics that relate to heart failure, such as characteristics that are indicative of apnea, Cheyne-Stokes Respiration, and/or periodic breathing.

In an embodiment of the present invention, breathing cycles are divided into successive segments of inspirium and expirium. Breathing pattern analysis module 22 interprets as indicative of an approaching or progressing attack a trend towards greater duration of the expirium segments in proportion to the inspirium during sleep (typically night sleep). In another embodiment, the duty cycle of breathing activity (duration of expirium plus inspirium segments) versus no respiratory motion is interpreted as an indicator of an approaching or progressing attack.

Reference is again made to FIG. 2. In an embodiment of the present invention, system 10 further comprises an acoustic sensor 110 for measurement of breathing-related sounds such as those caused by wheezing or coughing. (For some applications, in which breathing sensor 30 comprises a pressure gauge, acoustic sensor 110 is integrated with the pressure gauge. For example, a single sensor may be used for both acoustic sensing and measuring body motion. Alternatively, acoustic sensor 110 is a separate component.) Pattern analysis module 16 processes such breathing sounds independently, or time-locked to expirium and/or inspirium, e.g., by using spectral averaging to enhance the signal-to-noise ratio of wheezing sounds. For some applications, the level of wheezing and its timing with respect to the timing of inspirium and expirium provides additional information for predicting an upcoming asthma attack and/or monitoring the severity and progression of an attack. For example, for most patients, wheezing taking place during expiration is considered to be a more reliable indication of an asthma exacerbation than wheezing during inspiration.

Wheezing can be attributed to specific parts of the breathing cycle (mainly inspirium and expirium), and thus provides a useful insight regarding the type of upcoming or progressing respiratory distress. In addition, wheezing can be filtered according to the periodicity of the breathing cycle, thus enhancing identification of breathing-related sounds of the obstructed airways, and improving the ability to reject ambient noises that are not related to the breathing activity. Periodic, breathing-cycle-related wheezing can provide additional insight regarding the type of upcoming or progressing respiratory distress.

In an embodiment of the present invention, pattern analysis module 16 comprises cough analysis module 26, which is adapted to detect and/or assess coughing episodes associated with approaching or occurring clinical episodes. In asthma, mild coughing is often an important early pre-episode marker indicating an upcoming onset of a clinical asthma episode (see, for example, the above-mentioned article by Chang AB). In congestive heart failure (CHF), coughing may provide an early warning of fluid retention in the lungs caused by worsening of heart failure or developing cardiovascular insufficiency.

For some applications, coughing sounds are extracted from motion sensor 30 installed in, on, or under a reclining surface, typically using acoustic band filtering of between about 50 Hz and about 8 kHz, e.g., between about 100 Hz and about 1 kHz. Alternatively, the signal is filtered into two or more frequency bands, and motion data acquisition module 20 uses at least one frequency band of typically very low frequencies in the range of up to 10 Hz for registering body movements, and at least one other frequency band of a higher frequency range, such as between about 50 Hz and about 8 kHz, for registering acoustic sound. For some applications, the module uses a narrower acoustic band, such as between about 150 Hz and about 1 kHz.

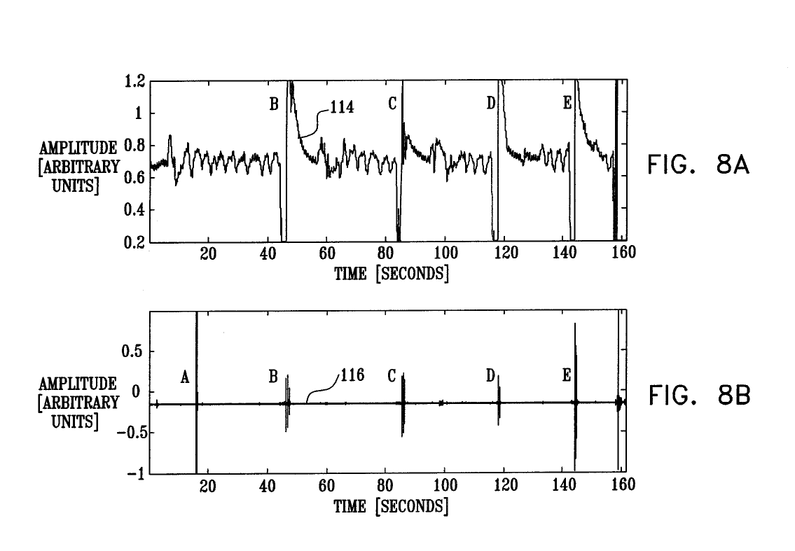

Reference is made to FIGS. 8A and 8B, which are graphs showing different frequency components of a motion signal, in accordance with an embodiment of the present invention. Coughing events comprise simultaneous body movement and bursts of non-vocal sounds followed by vocal sounds. Cough analysis module 26 extracts coughing events by correlating coughing signals from the acoustic signal with body movement signals from the motion signal. Typically, module 26 relies on both mechanical and acoustical components for positive detection of coughing events. FIG. 8A shows a low-frequency (less than 5 Hz) component 114 of the measured signal, and FIG. 8B shows a high-frequency (200 Hz to 1 kHz) component 116 of the measured signal. Cough analysis module 26 typically identifies as coughs only events that are present in both low- and high-frequency components 114 and 116. For example, high-frequency event A in component 116 is not accompanied by a corresponding low-frequency event in component 114. Module 26 therefore does not identify event A as a cough. On the other hand, high-frequency events B, C, D, and E in component 116 are accompanied by corresponding low-frequency events in component 114, and are therefore identified as coughs. For some applications, cough analysis module 26 utilizes techniques described in one or more of the above-mentioned articles by Korpas J et al., Piirila P et al., and Salmi T et al.

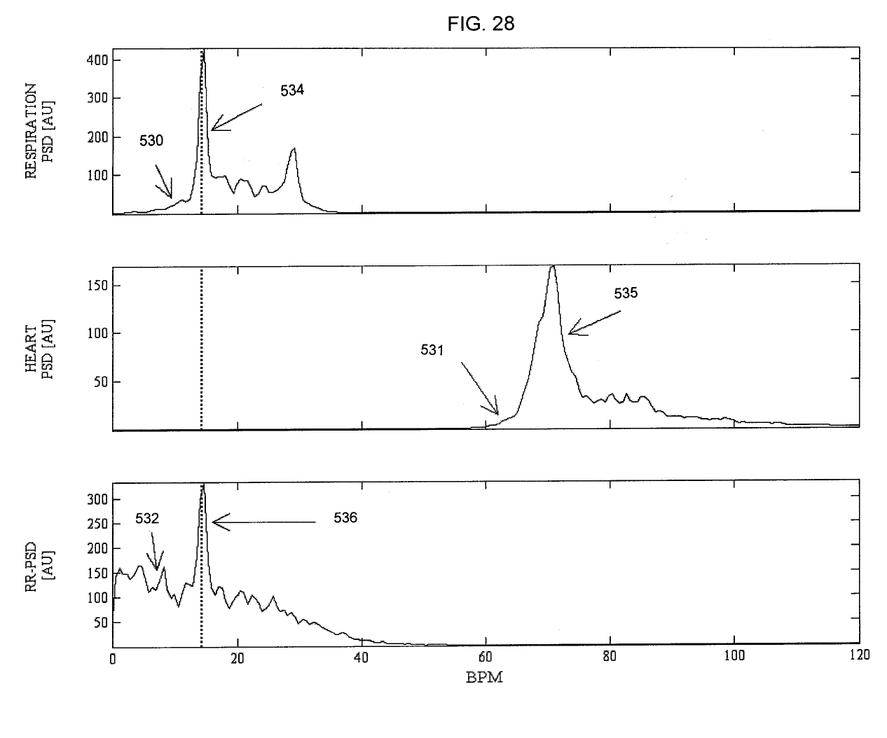

In an embodiment of the present invention, pattern analysis module 16 extracts breathing rate from a continuous heart rate signal using frequency demodulation, e.g., standard FM demodulation techniques. For example, the R-R interval is calculated by identifying the peaks of the heart beat signal using a standard peak detection algorithm. FIG. 26 shows the heartbeat signal as measured on an asthmatic child. FIG. 27 shows the R-R signal calculated from the heartbeat signal. FIG. 28 shows the power spectrum of the R-R signal (line 532) and the power spectrum of the respiration signal (line 530) both display a clear peak (peaks 534 and 536) corresponding to the respiration rate.

In another embodiment, the R-R signal is used in order to calculate the ratio of expiration to inspiration time of the subject. This ratio is indicative of the status of the subject's respiratory system. Due to sinus-arrhythmia, R-R intervals are expected to increase during expiration and decrease during inspiration. By calculating the ratio of the time the R-R signal is increasing to the time the R-R signal is decreasing and averaging over multiple cycles (to increase both accuracy and precision) the expiration to inspiration ratio is calculated.

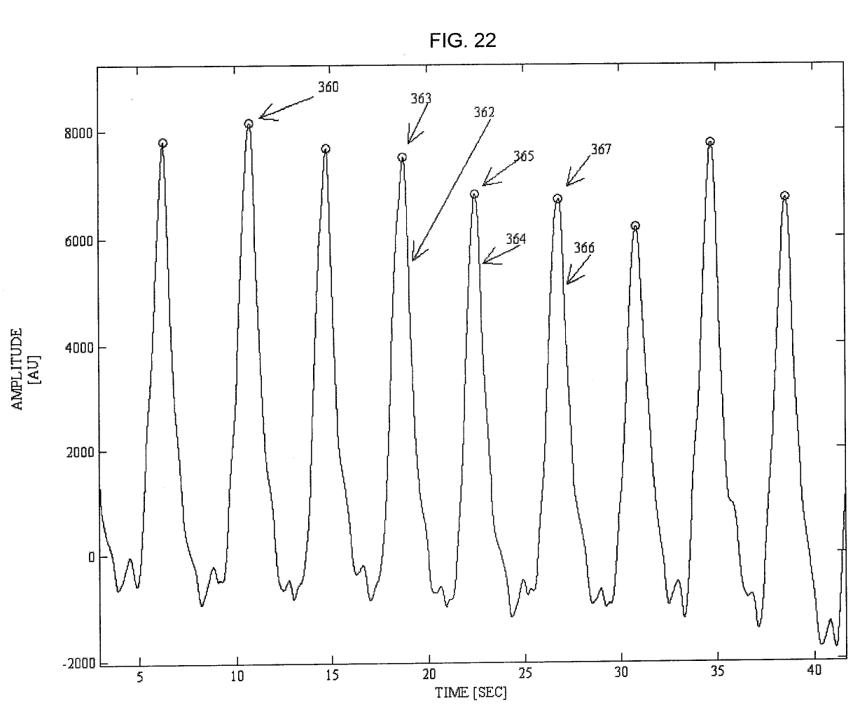

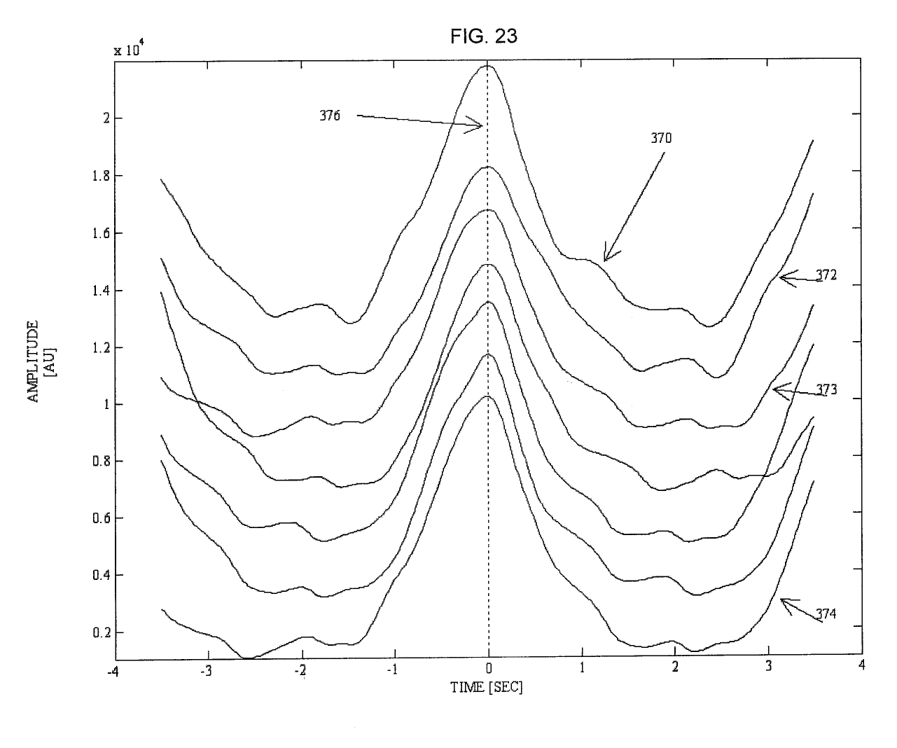

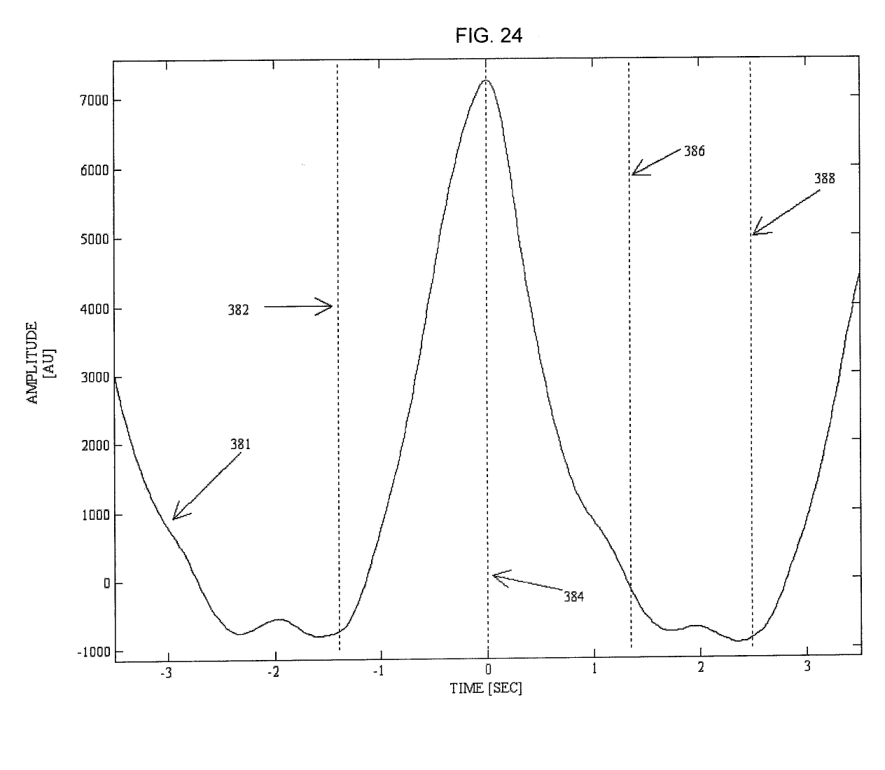

In another embodiment, principal respiration parameters such as duty cycle and expiration/inspiration ratio are extracted from the respiration related pressure signal. A normal respiration pattern is comprised of repeating signal complexes comprised of inspiration, respiration, and resting segments. Assuming signal stationarity over short time periods, as expected during most sleep stages, small inter-complex variations can be averaged out using synchronized ensemble averaging of aligned respiration signal complexes. Synchronized averaging is implemented utilizing signal peak attributes, corresponding to transition from inspiration to expiration, as alignment points. The resulting high-quality averaged respiration signal complex is used for identification of principal respiration parameters, where the rise-time indicates an inspiration segment, fall-time indicates an expiration segment, and the time period between the end of an expiration segment and the start of the next inspiration segment indicates a resting segment. Changes in respiration parameters such as inspiration/expiration segment ratios, shortening of resting periods and duty cycle, as well as changes in signal complex waveform, may be used for identification of an approaching asthma episode and to monitor the progression or remission of an ongoing episode. For example, FIG. 22 shows a mechanically measured respiration signal, with identified peaks 365, 366, and 367. FIG. 23 shows the respiration cycles of FIG. 22 aligned with each other according to the location of their peaks and shifted vertically for display purposes only. FIG. 24 shows the results of averaging the aligned respiration cycles of FIG. 23. Line 381 shows the average shape of the respiration cycle measured for that patient. The section of the cycle from 382 to 384 corresponds to the inspiration. The section from 384 to 386 denotes the expiration, and the section from 386 to 388 is the rest period.

In some embodiments, a mechanical sensor may display an inverted respiration signal. The correct orientation of the signal is received by either using the pulse signal. Thus the increased heart rate is expected during inspiration. Alternatively, the location of the rest period is used to identify the correct orientation since it is generally expected to appear after the expiration. This is possible because the heart rate signal generally displays a normal breathing-related sinus-arrhythmia pattern.

In an embodiment of the present invention, pattern analysis module 16 extracts breathing rate from a continuous heart rate signal using amplitude demodulation, e.g., using standard AM demodulation techniques. This is possible because respiration-related chest wall movement induces mechanical modulation of the heartbeat signal.

In an embodiment of the present invention, pattern analysis module 16 uses an amplitude- and/or frequency-demodulated heart rate signal to confirm adequate capture of the breathing and heart rate signals, by comparing the breathing rate signal with the demodulated sinus-arrhythmia pattern extracted from the heart-rate signal. For some applications, the sinus-arrhythmia pattern is frequency-demodulated by taking a series of time differences between successive heart beats, providing a non-biased estimate of the ongoing breathing pattern. Alternatively or additionally, the heart beat is amplitude-demodulated using high-pass filtering, full-wave rectification, and low-pass filtering.

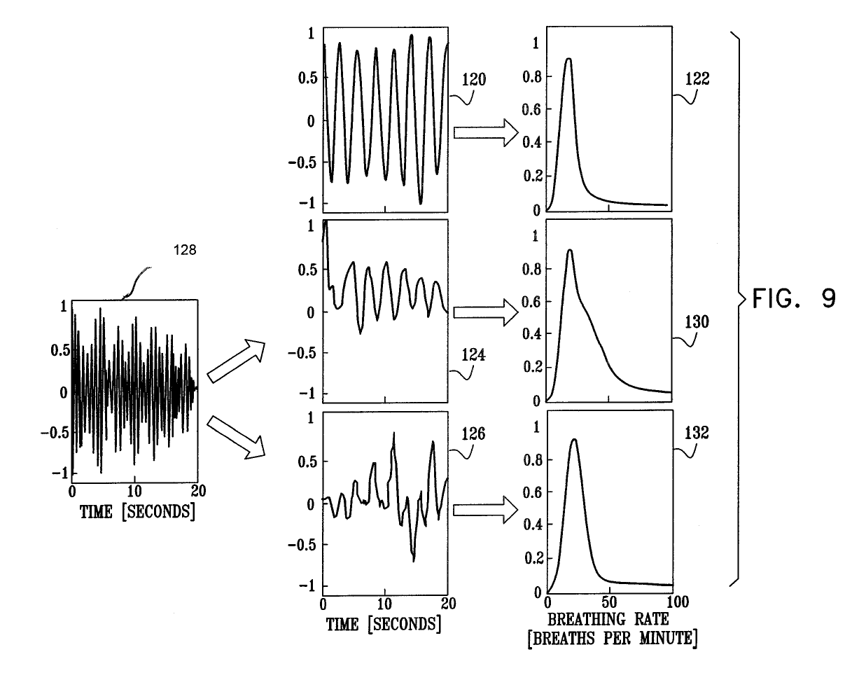

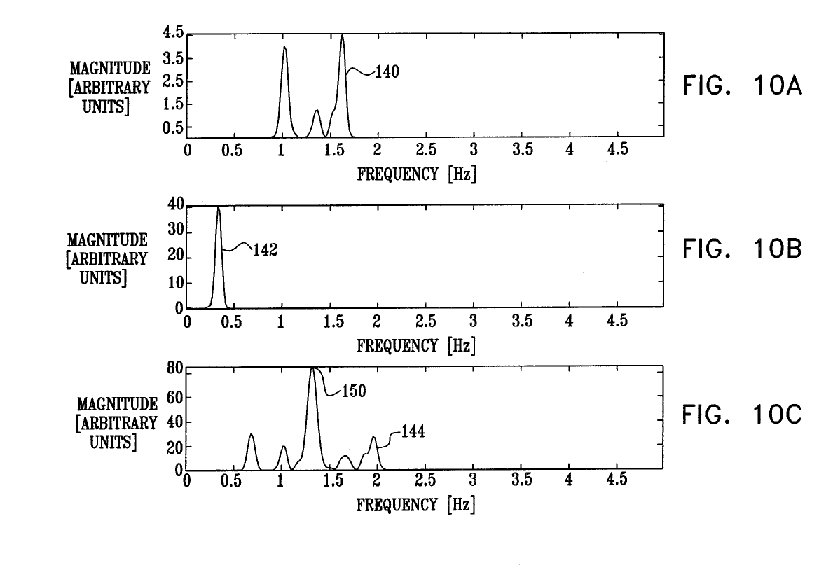

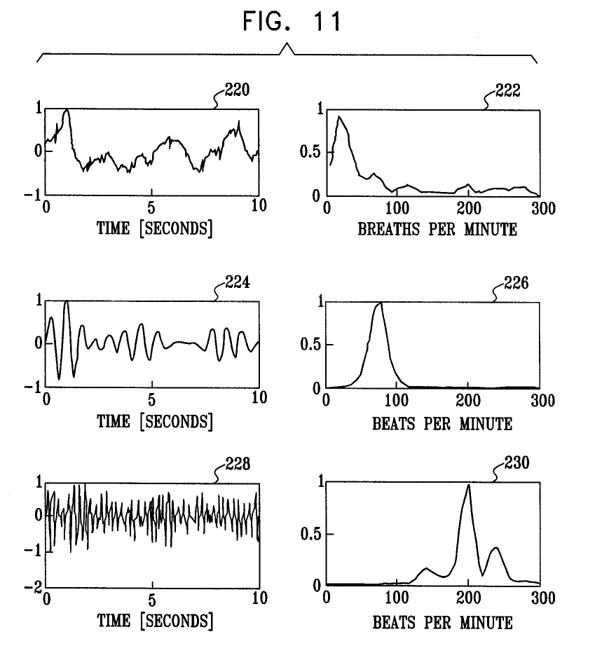

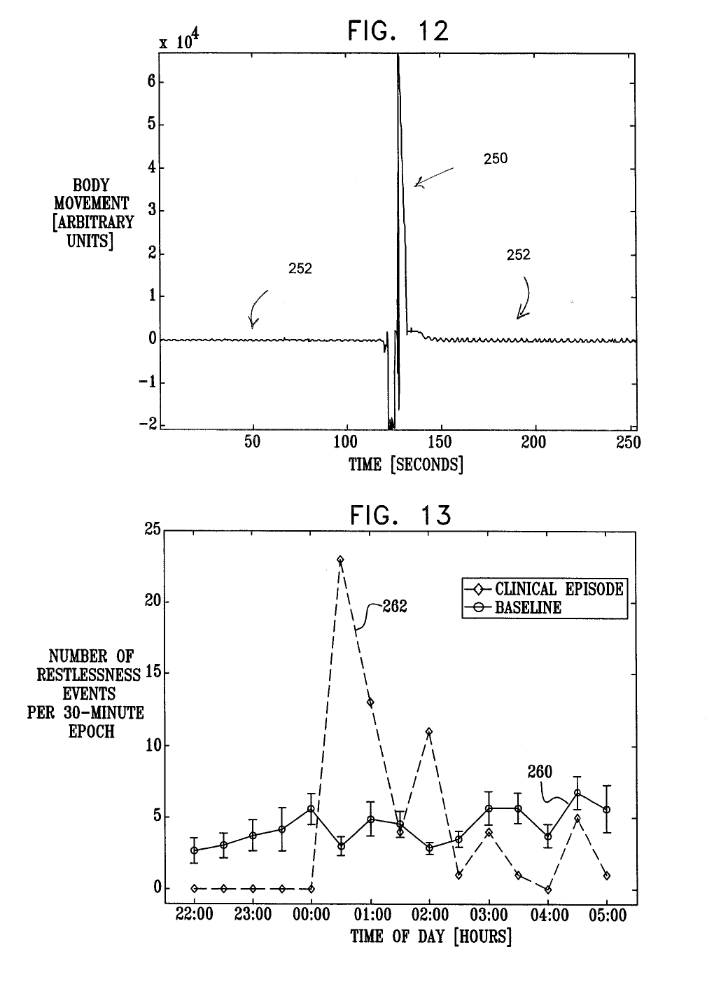

Reference is made to FIG. 9, which includes graphs showing several signals in time and corresponding frequency domains, in accordance with an embodiment of the present invention. Graphs 120 and 122 show a respiration signal in the time and frequency domains, respectively. Graphs 124 and 126 show amplitude-demodulated and frequency-demodulated respiratory patterns, respectively, both of which were derived from the heartbeat signal shown in a graph 128. Graphs 130 and 132 show the respiration signals derived from graphs 124 and 126, respectively, in the frequency domain.

These graphs demonstrate the similarity between (a) breathing rate pattern derived directly from a respiration signal, as shown in graphs 120 and 122, and (b) breathing rate pattern derived indirectly from a heartbeat signal, as shown in graphs 124, 126, 130, and 132. This similarity is particularly pronounced in the frequency domain, as shown in graphs 122, 130, and 132.

In an embodiment of the present invention, pattern analysis module 16 derives a heartbeat signal from a breathing-related signal. This approach may be useful, for example, if the breathing-related signal is clearer than the directly monitored heartbeat signal. This sometimes occurs because the breathing-related signal is generated by more significant mechanical body movement than is the heartbeat-related signal.

In an embodiment of the present invention, the measured breathing-related signal is used to demodulate the heartbeat-related signal and thus enable improved detection of the heartbeat-related signal. For some applications, breathing pattern analysis module 22 extracts breathing-related signals using spectral filtering in the range of about 0.05 to about 0.8 Hz, and heartbeat pattern analysis module 23 extracts heartbeat-related signals using filtering of in the range of about 0.8 to about 5 Hz. Heartbeat pattern analysis module 23 demodulates the heartbeat-related signal using the breathing-related signal, such as by multiplying the heartbeat-related signal by the breathing-related signal. This demodulation creates a clearer demodulated signal of the heart rate-related signal, thereby enabling its improved detection. In some cases, the power spectrum of the demodulated signal will show a clear peak corresponding to the demodulated heart rate.

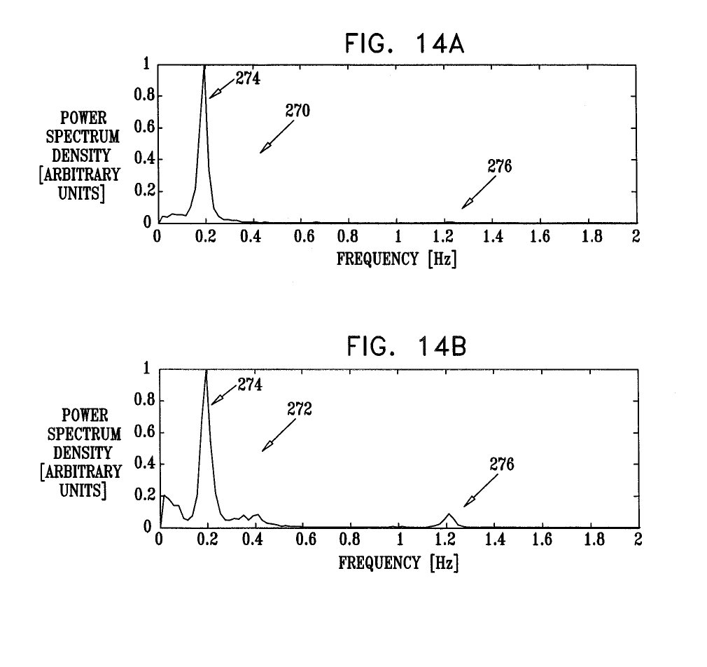

FIGS. 10A, 10B, and 10C are graphs showing frequency spectra, measured in accordance with an embodiment of the present invention. FIG. 10A shows a frequency spectrum signal 140 of a raw heartbeat-related signal (raw signal not shown), and FIG. 10B shows a breathing-related frequency spectrum signal 142, as measured simultaneously. FIG. 10C shows a demodulated spectrum signal 144 that is the product of breathing-related spectrum signal 142 (FIG. 10B) and heartbeat-related spectrum signal 140 (FIG. 10A). A clear peak 150 can be seen in demodulated spectrum signal 144, which represents the demodulated heartbeat frequency.

For some applications, the breathing-related signal used in the demodulation is filtered with a reduced top cut-off frequency (for example 0.5 Hz, instead of the 0.8 Hz mentioned above). Such a reduction generally ensures that only the basic sine wave shape of the breathing-related signal is used in the demodulation calculation.

In an embodiment of the present invention, breathing pattern analysis module 22 is configured to detect, typically during night sleep, an abnormal breathing pattern associated with CHF, such as tachypnea, Cheyne-Stokes Respiration (CSR), or periodic breathing.

In an embodiment of the present invention, system 10 is adapted to determine fetal heart rate. Typically, maternal heart rate in a relaxed setting is below 100 beats per minute (BPM), while healthy fetal heart rate is typically above 110 BPM. Heartbeat pattern analysis module 23 of system 10 distinguishes the fetal heart signal from the maternal heart signal, typically using lower pass-band filtering for the maternal heartbeat signal, and higher pass-band filtering to obtain the fetal heartbeat signal.

FIG. 11 includes graphs showing combined and decomposed maternal and fetal heartbeat signals, measured in accordance with an embodiment of the present invention. Graphs 220 and 222 show a measured combined maternal and fetal respiration and heart signal, in the time and frequency domains, respectively. The signal shown in graph 220 was decomposed into its two constituents: (1) maternal heart signal, shown in the time and frequency domains in graphs 224 and 226, respectively, and (2) fetal heart signal, shown in the time and frequency domains in graphs 228 and 230, respectively.

In an embodiment of the present invention, the maternal breathing signal is used to differentiate or confirm maternal heartbeat patterns by matching the maternal breathing pattern with the maternal heart sinus-arrhythmia pattern. This is possible because, as mentioned above, the maternal pulse is frequency- and amplitude-modulated by the maternal breathing rate. Confirmation that maternal heartbeat has been correctly identified enables the identification of fetal heartbeat pattern.

In an embodiment of the present invention, the maternal breathing-related signal (which is often stronger than the fetal heartbeat-related signal) is used to demodulate the fetal heartbeat-related signal. This is possible because in some cases the fetal heart rate signal is amplitude-modulated by the maternal respiration signal. In these cases, the maternal respiration signal, which is relatively easy to detect, is used to extract the fetal heart rate signal, which is relatively difficult to detect, from background noise. For example, the fetal heart rate signal may determined by: (1) determining the maternal respiration rate using techniques described hereinabove; (2) passing the motion signal through a band pass filter appropriate for fetal heart rate (e.g., about 1.2 Hz to about 3 Hz); (3) multiplying the filtered signal by the respiration signal; (4) performing a Fast Fourier Transform on the resulting signal; and (5) identifying a peak in the transformed signal as corresponding to the fetal heart rate.

In an embodiment of the present invention, system 10 is adapted to measure fetal motion patterns, which have an amplitude or frequency characteristic which is different from maternal movement. The signal generated by fetal motion is weaker than the signal generated by maternal motion, and has a higher frequency (when analyzed in the frequency domain) than the signal generated by maternal motion. In addition, fetal motion is generally registered primarily (or at least most strongly) by the abdominal sensors, while maternal motion is generally registered both by the abdominal sensors and other sensors (e.g., leg sensors). For some applications, system 10 comprises a plurality of motion sensors 30, and system 10 monitors high frequency movement in the vicinity of the mother's abdomen, in order to identify and count fetal movements.

In an embodiment of the present invention, system 10 is configured to monitor sleep cycles by monitoring cardiac and respiratory data, and to identify that a sleeping user is in an optimal sleep stage for awakening, such as light sleep or REM sleep. Upon detection of such sleep stage during a user-selected timeframe for awakening, system 10 drives user interface 24 to generate a visible and/or auditory signal to awaken the user. For some applications, techniques described in the above-mentioned article by Shinar Z et al. are used for obtaining sleep staging information from respiration and heart rate data, mutatis mutandis. In this embodiment, motion sensor 30 is typically installed in, on, or under reclining surface 37 (FIG. 1). For some applications, only certain components of system 10 are used, rather than the complete system, such as motion data acquisition module 20, motion sensor 30, breathing pattern analysis module 22, and/or heartbeat pattern analysis module 23 (FIG. 2).

In an embodiment of the present invention, system 10 performs continuous monitoring and registration, on a night-to-night basis, of multi-sign data, including life signs and auxiliary signs, such as breathing patterns, heartbeat patterns, movement events, and coughing. The registered multi-sign data is used to construct a personalized patient file, which serves as a reference for tracking of pathophysiological deviations from normal patterns.

In an embodiment of the present invention, a plurality of measured parameters are combined using the following formula:
F=A1*ΔP1+A2*ΔP2+ . . . +An*ΔPn (Equation 1)
where Ai is the relative weight given to parameter Pi, and ΔPi is the difference between the value of Pi for a given night and a baseline value defined for Pi. F is typically calculated on an hourly or a nightly basis and compared to a reference value that is predefined or determined based on personal history. If the value of F exceeds the reference value, the system alerts the subject and/or a healthcare worker. As appropriate for any of the parameters Pi, the absolute value of ΔPi may be evaluated, instead of the signed value of ΔPi. As appropriate for any of the parameters Pi, the square, square root, exponential, log, or any other similar function may be evaluated. Alternatively or additionally, for any of the parameters Pi, instead of using ΔPi, a value generated by inputting ΔPi into a lookup table is used. Further alternatively or additionally, the resulting function F is entered into a lookup table (either predefined or learned) in order to interpret the result.

In an embodiment of the present invention, a plurality of parameters is combined by calculating a score for each parameter and applying a function to combine the scores, such as Equation 1. For some applications, each score represents a probability of an occurrence of the value of the parameter if a clinical episode is not imminent within a certain time period, e.g., within the next 1 hour, 4 hours, 24 hours, or 48 hours. The function estimates a combined probability of an occurrence of the values of the parameters in combination if the clinical episode is not imminent within the time period. For example, for n monitored parameters, each with a respective threshold t(i), and a probability p(i) of crossing threshold t(i) when a clinical episode is not imminent, a binomial distribution is calculated to indicate the probability that an observed combination of threshold crossings is random. If the probability of observing the combination is low, then an alarm signal is generated or other action taken. For example, probability of observing the combination may be compared to a threshold that is either predefined or learned by system 10. If the probability is less than the threshold, system 10 generates an alarm indicating that there is a high probability than an episode is imminent. For some applications, the scores for each parameter are weighted, as described above with reference to Equation 1.

In an embodiment of the present invention, system 10 is adapted to learn the above-described thresholds, weights, and/or probabilities. For some applications, system 10 uses the following method for performing such learning:
upon each occurrence of an episode, the subject or a healthcare worker enters an indication of the occurrence of the episode into system 10 via user interface 24. Alternatively or additionally, the system itself identifies an episode by detecting parameters clearly indicative of an episode (e.g., a respiration rate of over 30 breathers per minute). Further alternatively, system 10 determines that an episode has occurred based on input from drug administration device 266 (e.g., the system interprets a level of usage of an inhaler beyond a certain threshold as indicative of an occurrence of an episode).
from time to time (e.g., once every two weeks), system 10 compares actual episodes with episodes about which the system provided a warning;
for each correctly predicted episode, false negative, and false positive, the system checks the accuracy of the prediction given by the system according to the current thresholds, weights, and probability distribution; and
responsively to this check, the system incrementally adjusts one or more of the thresholds, weights, or probability distributions.

For example, some asthma patients have coughs that precede their attacks, while other patients do not. Every two weeks, the system checks whether cough symptoms occurred prior to each attack. The system accordingly adjusts the threshold up or down by a certain percentage (e.g., 5%) for each false positive or false negative. For example, for some applications, for each correctly predicted attack, the system adjusts the weight of the cough parameter (for example, if there was substantial coughing prior to the most recent five attacks, the system increases the weight of the cough parameter). Alternatively or additionally, the system may adjust the weight of the coughing parameter for false positives or false negatives.

In an embodiment of the present invention, system 10 monitors and analyzes episodes of nocturnal restlessness and/or awakening, which are symptoms of several chronic conditions, such as asthma and CHF. Typically, system 10 quantifies these episodes to provide an objective measure of nocturnal restlessness and/or awakening. As described hereinabove, system 10 analyzes a cyclical motion signal of the subject in the frequency domain, and identifies peaks in the frequency domain signal corresponding to respiration rate and heart rate (and, optionally, corresponding harmonics). Body motion of the subject generates a sudden, generally stronger non-cyclical component in the motion signal. System 10 interprets an occurrence of such non-cyclical motion to be a restlessness episode if such motion is transient (e.g., has a duration of between about 2 and about 10 seconds), after which the periodic respiration/heart beat signal returns. System 10 interprets an occurrence of such non-cyclical motion to be an awaking event if such motion continues for more than a certain period of time, or if there is no periodic signal for more than a certain period of time (both of which conditions indicate that the subject is no longer in bed).

In an embodiment of the present invention, system 10 monitors and analyzes episodes of nocturnal restlessness and/or awakening, which are symptoms of several chronic conditions, such as asthma and CHF. Typically, system 10 quantifies these episodes to provide an objective measure of nocturnal restlessness and/or awakening. As described hereinabove, system 10 analyzes the motion signal of the subject in the frequency domain, and identifies peaks in the frequency domain signal corresponding to respiration rate and heart rate (and, optionally, corresponding harmonics). Body motion of the subject generates a sudden, generally stronger non-cyclical component in the motion signal. System 10 divides the monitored period into time epochs of a duration that includes several respiration cycles, typically between 30 and 300 seconds, for example 60 seconds. Each epoch is identified as ‘quiet’ or ‘noisy’. An epoch is identified as quiet if its power spectrum has a peak in the range expected for respiration for that subject (e.g. 0.2-0.5 Hz). The standard deviation of the mechanical signal is calculated for each quiet epoch. The restlessness level is calculated as follows: initially system 10 defines a threshold level for each time epoch. The threshold is defined, for example, in reference to the standard deviation of the data in a ‘quiet’ epoch and is valid for the consecutive ‘noisy’ epochs. For example, the threshold is defined as 2-10 times the standard deviation, for example 3 times the standard deviation. For each time epoch, the area of the mechanical data signal above the corresponding threshold estimates the restlessness of that duration as shown in the digital integration method in Ancoli-Israel S, Cole R, Alessi C et al. in the American Academy of Sleep Medicine Review Paper in SLEEP 2003;26(3):342-92.

Another indication of respiratory pattern change is the existence of enhanced respiration movements such as: augmented breaths (sighs) and deep inspirations, for example, as described by Hark et al. in Ann Allergy Asthma Immunol. 2005 February;94(2):247-50 and by Delmore and Koller in Pflugers Arch. 1977 Nov. 25;372(1):1-6 and Kaspali, et al. in the Journal of Applied Physiology, August 2000, 89: 711-720. In an embodiment of the present invention, system 10 monitors and analyzes events of augmented breaths (also known as ‘sighs’) and deep inspirations. Typically, system 10 quantifies these events and measures their number and rate at different segments of the night and in some cases in different sleep stages. This serves as an additional clinical parameter for the evaluation of the patient's clinical status. An event of deep inspiration or sigh is calculated as follows: initially the end-inspiration and end-expiration times are located (similar to R wave detection on ECG signals). From these two parameters the breathing length (time between two successive end-inspiration events) and breathing depth (respiration amplitude at end-inspiration minus respiration amplitude at end-expiration) are calculated. A breathing cycle is defined as a sigh/augmented breath or deep inspiration if it is significantly deeper than a normal respiration cycle and for example, the following requirements occur: 1) the depth is between 1.5-3 times the average depth of nearest 12 cycles, 2) the length is between 1-2 times the averaged length of nearest 12 cycles, and 3) the standard deviations of the length and of the depth of nearest 12 cycles is less than 20%.

In another embodiment, system 10 is used to differentiate between sigh dyspnea and asthma.

Some asthma patients take short-term medication on an extensive basis much more than recommended by healthcare professionals. In some cases, for example teenaged patients, this is done in an irresponsible manner and without reporting to the parent, guardian, or healthcare professional. In some cases excessive use of such medication, e.g. bronchodilators, reduces the effectiveness of treatment and may result in an insufficient relief in case of asthma emergency. There is therefore a need to identify excessive use of bronchodilators. Bronchodilators have a characteristic effect on heart rate and respiration rate that usually subsides within 4-6 hours. In one embodiment the system identifies this pattern and logs the number and dates of apparent use of bronchodilators. It then informs the patient, caregiver, or healthcare professional of the usage statistics of the bronchodilators.

Patients with sleep apnea are often treated with Continuous Positive Airway Pressure (CPAP) systems. In many cases it is beneficial to sense the respiration rate and heart rate in order to optimize the use of CPAP devices. In one embodiment of the present invention, motion data acquisition module 20 extracts breathing-related signals by performing spectral filtering in the range of about 0.05 to about 0.8 Hz, and heartbeat-related signals by performing spectral filtering in the range of about 0.8 to 5.0 Hz. The respiration rate and heart rate patterns as well as, in some cases, other clinical parameters measured by system 10 are used to optimize the operation of the CPAP device.

Reference is made to FIG. 12, which is a graph showing body movement, in accordance with an embodiment of the present invention. In this embodiment, system 10 monitors restlessness manifested by excessive body movement during sleep. System 10 quantifies the restlessness to provide an objective measure of nocturnal restlessness. As seen in FIG. 12, a restlessness event 250 is characterized by a substantial increase in body movement, compared to normal sleep periods 252. In this embodiment, motion sensor 30 is typically installed in, on, or under reclining surface 37 (FIG. 1). For some applications, system 10 classifies a time segment as indicative of restlessness when the standard deviation of the measured motion signal during the time segment is at least a certain multiple of the average standard deviation of the motion signal during at least a portion of the sleep period. For example, the multiple may be between about 2 and about 5, such as about 3. Alternatively, system 10 uses other mathematical and/or statistical indicators of deviation, such as the frequency domain analysis techniques described above. Alternatively, system 10 uses an integrator function J(i) which is defined by the following equation:
J(i)=(1-alpha)*J(i-1)+alpha*abs(X(i)) (Equation 2)
where X(i) is the raw signal as sampled from motion sensor 30. If for example, X(i) has 10 samples per second, appropriate values for alpha would be between 0.01 and 0.1, e.g., 0.05. The signal J is typically averaged for the whole night, and a standard deviation is calculated. If at any point, J(i) exceeds the average by more than two times the standard deviation for a period lasting at least 2 seconds, a restlessness event is defined.

For some applications, once such restlessness events are identified, system 10 counts the number of events per time epoch (for example, each time epoch may have a duration of 30 minutes). To detect a clinical episode (such as of any of the conditions described herein), system 10 compares measured night patterns with a reference pattern, according to certain criteria. For example, system 10 may generate a clinical episode warning if a restlessness event is detected in more than a certain percentage of time epochs (e.g., more than 10%, 20%, or 30%). Alternatively, system 10 generates a clinical episode warning if the total number of restlessness events per night exceeds a threshold value. For some applications, the reference pattern or threshold value is determined based on population averages, while for other applications, the reference pattern or threshold value is determined by averaging the data from the subject over several non-symptomatic nights.

Reference is made to FIG. 13, which is a graph showing restlessness events during normal sleep and during a clinical episode of asthma, in accordance with an embodiment of the present invention. A line 260 shows the number of restlessness events per 30-minute epoch during normal sleep (the bars indicate standard error). A line 262 shows the number of restlessness events per 30-minute epoch during a night characterized by a clinical episode of asthma.

In an embodiment of the present invention, system 10 monitors episodes of arousal because of general restlessness or coughing, in order to provide additional evidence for certain pathologies such as an approaching or progressing asthma episode.

In an embodiment of the present invention, system 10 records monitored parameters such as respiration, heart rate, and/or coughing during sleep at night. The system analyzes the recorded parameters either continuously or after the conclusion of sleep, such as in the morning, to predict an approaching clinical episode. In the morning, or later in the day, system 10 drives user interface 24 to alert the subject about the approaching clinical event. Such approaching clinical events generally do not occur until at least several hours after system 10 predicts their approach, such as at least 12 or 24 hours. Therefore, delaying notification until the morning or later in the day still generally provides sufficient time for the subject to begin preventive treatment before clinical manifestation of the episode begins, without needlessly interrupting the subject's sleep. For some applications, system 10 analyzes the parameters to estimate a severity and/or urgency of the approaching clinical episode, and to determine whether to wake the subject responsively to the severity and/or urgency.

For applications in which system 10 detects worsening of a clinical episode already in progress, or that an episode will begin within a relatively short period of time (e.g., within four hours), system 10 provides a warning without delay to enable fast treatment of the worsening episode. In addition, system 10 typically records and continuously analyzes monitored parameters throughout sleep.

In an embodiment of the present invention, system 10 is configured to detect episodes of pulse irregularity, such as during ventricular fibrillation or cardiac arrest, and to provide an immediate alert upon detection of such an irregularity. Alternatively or additionally, upon detection of such an irregularity, system 10 automatically administers an appropriate electric or magnetic shock. For example, user interface 24 may comprise an implantable or external cardioverter/defibrillator, as is known in the art.

In an embodiment of the present invention, motion sensor 30 and all or a portion of motion data acquisition module 20 are packaged in a biocompatible housing (or in multiple housings) adapted to be implanted in subject 12. The implantable components comprise a wireless transmitter, which is adapted to transmit the acquired signals to an external receiver using a transmission technology such as RF (e.g., using the Bluetooth® or ZigBee protocols, or a proprietary protocol) or ultrasound. Alternatively, one or more of analysis modules 22, 23, 26, 28, 29, or 31, and/or user interface 24 are also adapted to be implanted in the subject, either in the same housing as the other implantable components, or in separate housings. Further alternatively, motion sensor 30 is adapted to be implanted in subject 12, while motion data acquisition module 20 is adapted to be external to the subject, and in communication with motion sensor 30 either wirelessly or via wires.

In an embodiment of the present invention, user interface 24 is configured to accept input of information regarding medical treatment the subject is currently receiving, such as drug and dosage information. Prophylactic or clinical pharmacological treatments may affect physiological parameters such as respiration, heart rate, coughing, and restlessness. For example, respiration patterns of asthma patients may be affected by usage of bronchodilator medication. Pattern analysis module 16 therefore takes the entered information into account when assessing deviations of measured parameters from baseline parameters. For example, breathing pattern analysis module 22 may disregard a slight increase of about 10% in respiration rate compared to baseline if the increase occurs within about one hour after use of bronchodilator medication and lasts up to 8 hours thereafter.

Reference is again made to FIG. 2. For some applications, drug treatment information is directly transmitted to system 10 from a drug administration device 266, rather than manually entered into user interface 24. Such drug information treatment may include, for example, which drug has been administered (and/or the drug's active ingredients), the dosage of the administered drug, and/or the timing of the administration. For some applications, system 10 takes the drug treatment information into account when determining the dosage and/or drug administration timing information that the system provides to drug administration device 266. Transmission of data to system 10 may be performed wirelessly or via wires. For example, drug administration device 266 may comprise a commercially-available drug administration device having communication capability, such as the Nebulizer Chronolog (Medtrac Technologies, Inc., Lakewood, Colo., USA), or the Doser (MEDITRACK Products, Hudson, Mass.).

In an embodiment of the present invention, system 10 automatically detects and extracts parameter pattern changes related to a specific pharmacological treatment, and considers the extracted pattern changes in assessment of parameter deviation from baseline patterns. For example, an increase of about 10% in respiration rate of an asthma patient, followed by a return to normal after about 6 to 8 hours, may be identified by system 10 as being associated with use of a bronchodilator.

Reference is yet again made to FIG. 2. In an embodiment of the present invention, system 10 is used in an automatic closed-loop with drug administration device 266. The drug administration device delivers a drug to subject 12. System 10 monitors the clinical effect of the drug, and provides feedback to the drug administration device to maintain or update the drug dosage. For some applications, drug administration device 266 comprises one or more of the following: a nebulizer, an inhaler, a vaporizer (e.g., in a room in which the subject is), a continuous positive airway pressure device, a spraying system, or an intravenous drug administration system. Alternatively or additionally, system 10 is configured to determine the optimal level of humidity in the room in which the subject is, in order to optimize one or more physiological parameters of the subject, and to drive a vaporizer or other humidifying device to appropriately control the humidity. Further alternatively or additionally, system 10 is configured to determine the optimal room temperature, in order to optimize one or more physiological parameters of the subject, and to drive an air conditioner and/or heater to appropriately control the temperature.

For some applications, drug treatment information is directly transmitted to system 10 from drug administration device 266, rather than manually entered into user interface 24. Such drug information treatment may include, for example, which drug has been administered (and/or the drug's active ingredients), the dosage of the administered drug, and/or the timing of the administration. For some applications, system 10 takes the drug treatment information into account when determining the dosage and/or drug administration timing information that the system provides to drug administration device 266.

For some applications, drug administration device 266 regulates the dosage of several drugs. For example, the drug administration device may regulate the dosage of drugs belonging to one or more of the following categories: bronchodilators, anti-inflammatories, antibiotics, and placebos. For some applications for treating asthma patients, drug administration device 266 comprises a metered-dose inhaler (MDI) comprising three chambers holding several types of drugs, such as a bronchodilator, an anti-inflammatory agent, and a placebo. When subject 12 wakes up in the morning, system 10 determines the current condition of the subject, and, responsively thereto, determines the appropriate dosage combination of the three drugs. System 10 communicates this dosage information to the MDI, which prepares the relevant combination to be inhaled. The subject activates the MDI for automatic administration of the appropriate combination and dosage of medications. These techniques obviate the need for the subject to know or control the drug combination delivered by the MDI. The techniques described in this paragraph are also appropriate for drug administration devices other than MDIs.

Reference is made to FIGS. 14A and 14B, which are graphs showing power spectrum densities of signals measured in accordance with an embodiment of the present invention. Lines 270 and 272 in FIGS. 14A and 14B, respectively, show the power spectrum density of signals measured under the abdomen and the legs, respectively. Peaks 274 and 276 correspond to the subject's respiration rate and heart rate, respectively. As can be seen in the graphs, for some applications heart rate is more clearly detectable in the signal measured under the legs.

Reference is again made to FIG. 2. In an embodiment of the present invention, system 10 comprises a temperature sensor 380 for measurement of body temperature. For some applications, temperature sensor 380 comprises an integrated infrared sensor for measurement of body temperature. Body temperature is a vital sign indicative of general status of systemic infection and inflammation. Global rise in body temperature is used as a first screening tool in medical diagnostics.

In an embodiment of the present invention, system 10 is configured to identify early signs of an onset of hypoglycemia in a diabetic subject. The system identifies an increase in a level of physiological tremor as being indicative of such onset, and/or an increase in the level of tremor in combination with other parameters described hereinabove, such as heart rate, respiration rate, and/or awakenings, and/or a change in the heart beat pattern indicative of palpitations (by analyzing the timing between peaks of the heart beat signal, using techniques described herein). Typically, the system detects physiologic tremor by monitoring body motion at between about 4 Hz and about 18 Hz, such as between about 8 Hz and about 12 Hz. Alternatively, the system identifies the increase in the level of physiological tremor as being indicative of an onset or progression of a condition selected from the list consisting of: Parkinson's disease, Alzheimer's disease, stroke, essential tremor, epilepsy, stress, fibrillation, and anaphylactic shock. For some applications, system 10 is adapted to drive user interface 24 to display one or more properties of the detected physiological tremor, such as an amplitude or spectrum image of the tremor. For example, system 10 may be used as a bedside hospital vital signs diagnostic system. For some applications, the hypoglycemia is identified by analyzing the heart signal to identify palpitations. Palpitations are identified as an increase in the heart rate and/or an increase in the irregularity of the heart beat (patients often characterize palpitations as “missing heart beats”).

In an embodiment of the present invention, system 10 monitors a subset of the physiological parameters described hereinabove, such as respiration rate, heart rate, cough count, blood pressure changes, expiration/inspiration ratio, respiration harmonics ratio, and tremor at multiple time points during the night. Pattern analysis module 16 assigns a score to each monitored parameter, and combines the scores to derive a compound score. The following is an exemplary formula for such a combination:
Combined Score=Const1*(Average Night Heart Rate−Baseline Heart Rate)+Const2*(Average Night Breathing Rate−Baseline Breathing Rate)+Const3*(Number of Night Coughs)+Const4*(Average Breathing Rate in Hour3−Average Breathing Rate in Hour2) (Equation 3)

Pattern analysis module 16 compares the combination score to a first threshold and a second threshold greater than the first. If the combination score is between the first and second thresholds, system 10 generates an alarm indicative of a future predicted clinical episode. If the combination score is greater than the second threshold, the system generates an alarm indicative of a currently occurring clinical episode. Alternatively, the scores and combination scores are vectors.

For some applications, these techniques are used in conjunction with the zone disease management methodology widely used by asthma patients, in which a “green” zone indicates no asthma symptoms, a “yellow” zone indicates a low level of attack, and a “red” zone indicates a high level of attack. System 10 drives user interface 24 to generate a green zone indication if the combination score is less than the first threshold, a yellow zone indication if the combination score is between the first and second thresholds, and a red zone indication if the combination score is greater than the second threshold.

For some applications, system 10 is configured to wake the subject from night sleep with an immediate alert if the combination score is greater than the second threshold, and to wait until morning to notify the subject if the combination score is between the first and second thresholds. The immediate alert may include an alarm sound and/or a light. A message which notifies the subject in the morning of a predicted onset of symptoms may be initially outputted from a user interface at any time after calculation of the combination score, in a manner that does not awaken the subject.

For some applications, system 10 is adapted to learn one or both of the thresholds, one or more of the parameters, and/or one or more of the constants used to generate the combination score. Techniques described hereinabove for such learning may be used.

In an embodiment of the present invention, system 10 comprises a plurality of motion sensors 30, such as a first sensor in a vicinity of abdomen 38 or chest 39 (FIG. 1), and a second sensor in a vicinity of legs 40. Pattern analysis module 16 determines the time delay between the pulse signal measured in the sensor under the abdomen or chest and the pulse signal measured under the legs. For example, the module may measure the time delay by performing a cross correlation between the heartbeat signals using a time window less than the respiration cycle time, such as between about 1 and 3 heart beat cycles. Alternatively, the module may identify the peaks in the heartbeat signals, and calculate the time differences between the peaks in each signal. Module 16 uses the time difference to calculate a blood pressure change signal on a continuous basis, for example as described in the above-mentioned U.S. Pat. No. 6,599,251 to Chen et al., mutatis mutandis. Module 16 calculates the amplitude in the change in blood pressure over a full inspiration/expiration cycle, and compares the amplitude to a threshold, such as 10 mmHg, or to a baseline value, either previously measured for the subject or based on a population average. Module 16 interprets an amplitude greater than the threshold as indicative of pulsus paradoxus. Alternatively or additionally, the system displays the amplitude and/or logs the amplitude to form a baseline for the specific patient which is later used to identify a change in condition.

In some cases, an increase in the average delay of the heart beat from the area of the heart to the extremities of the limbs is used as an indication of a deterioration in heart performance.

Some embodiments described herein relate to a set of vital signs and physiological behaviors that are monitored in order to predict and/or monitor clinical episodes. In some cases, it is useful to combine some of these capabilities to improve the monitoring and/or prediction capabilities of system 10, for example, for detecting the onset of hypoglycemia in a diabetic patient, as described hereinabove.

In an embodiment of the present invention, system 10 is adapted to count the number of arousals during a night. For some applications, such a count serves as an indication for the onset of asthma attacks, diabetes deterioration (e.g., waking up to drink water), small bowel and/or colon related diseases, or prostate problems (e.g., waking up to urinate). In an embodiment, the identification of arousals is performed using techniques described hereinabove, and/or in the above-referenced article by Shinar Z et al. (1998).

In an embodiment of the present invention, system 10 is adapted to monitor a geriatric subject, typically without contacting or viewing the subject or clothes the subject is wearing. For example, system 10 may be configured to monitor one or more of respiration rate, heart rate, coughs, sleep time, wake up events, and restlessness in sleep. For some applications, system 10 analyzes one or more of these parameters to determine when the subject is attempting to get out of bed without assistance, and notifies a healthcare worker. Death or injury is often caused by patients' attempts to get out of bed without assistance.

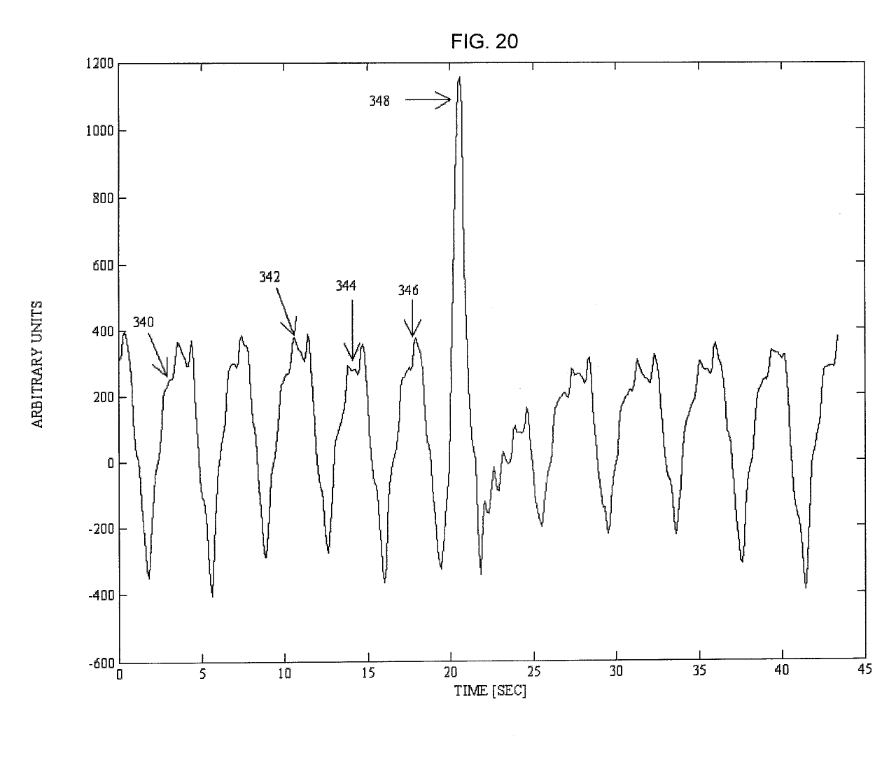

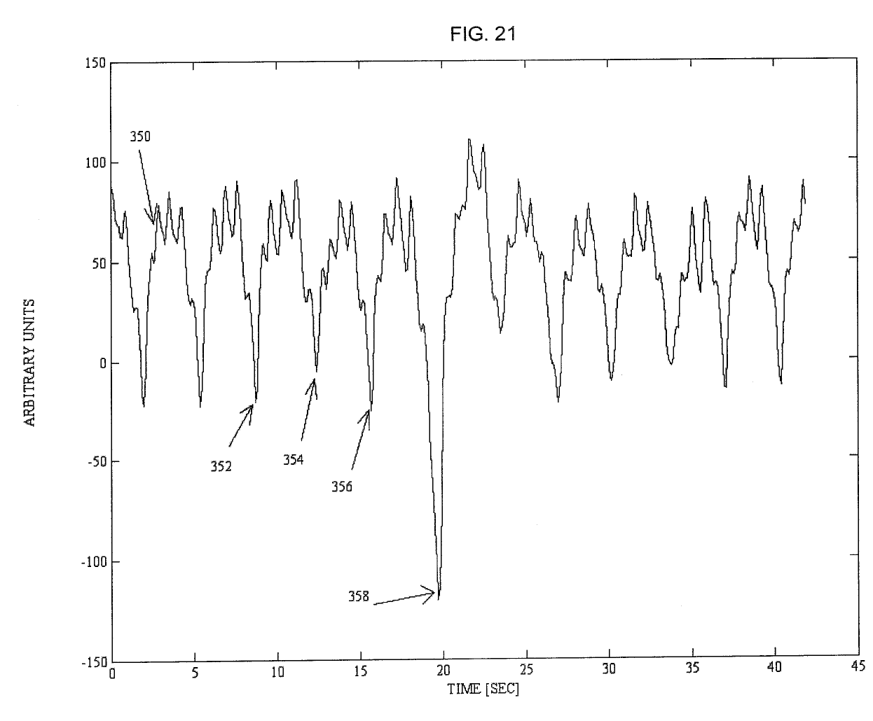

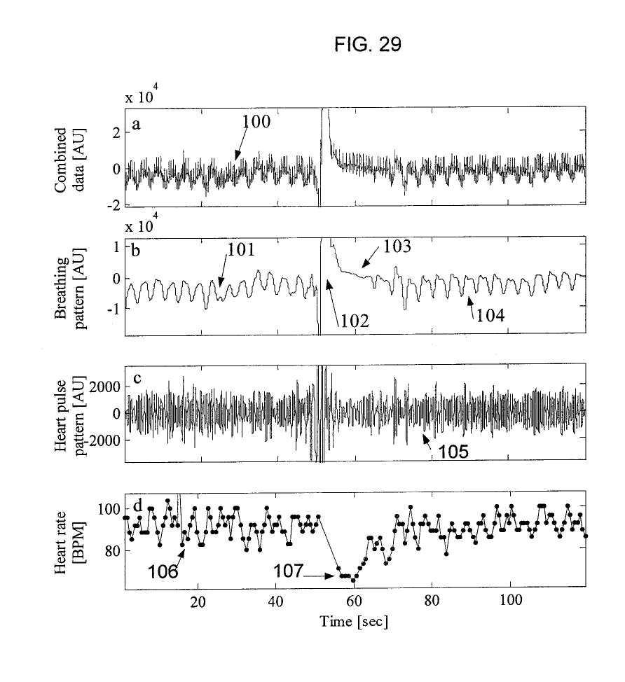

In an embodiment of the present invention, system 10 is adapted to monitor breathing and pulse (or heartbeat) patterns in order to recognize Central Sleep Apnea (CSA) episodes. FIGS. 29A-D illustrate an example of a CSA episode, as recorded by system 10, obtained from a 7-year-old asthmatic patient during the night. FIG. 29A shows the combined breathing and pulse signals (line 100), for example, as detected by motion sensor 30 in FIGS. 1 and 2. The corresponding breathing pattern extracted from the combined signal 100 is shown in FIG. 29B. Note that the quiet and steady breathing pattern 101 that is followed by a single deep breath cycle 102 and then a 18.7 second interval with no breathing effort, epoch 103, and finally, the breathing pattern returns to normal, epoch 104. Line 105 in FIG. 29C denotes the heart pulse or heartbeat signal derived from the combined signal 100 shown in FIG. 20A. The corresponding beat-to-beat heart rate is shown in FIG. 29D and denoted by line 106. Note the immediate decrease in heart rate during the CSA episode, epoch 107.

Obstructive sleep apnea (OSA) is a disorder in which complete or partial obstruction of the airway during sleep occurs due to a collision of the pharynx into the upper airway that blocks breathing. As a result, the patient suffers from loud snoring, oxyhemoglobin desaturations and frequent arousals. These arousals may occur hundreds of times each night but do not fully awaken the patient, who remains unaware of the loud snoring, choking, and gasping for air that are typically associated with obstructive sleep apnea. In contrast to central sleep apnea, OSA includes futile inspiratory efforts.

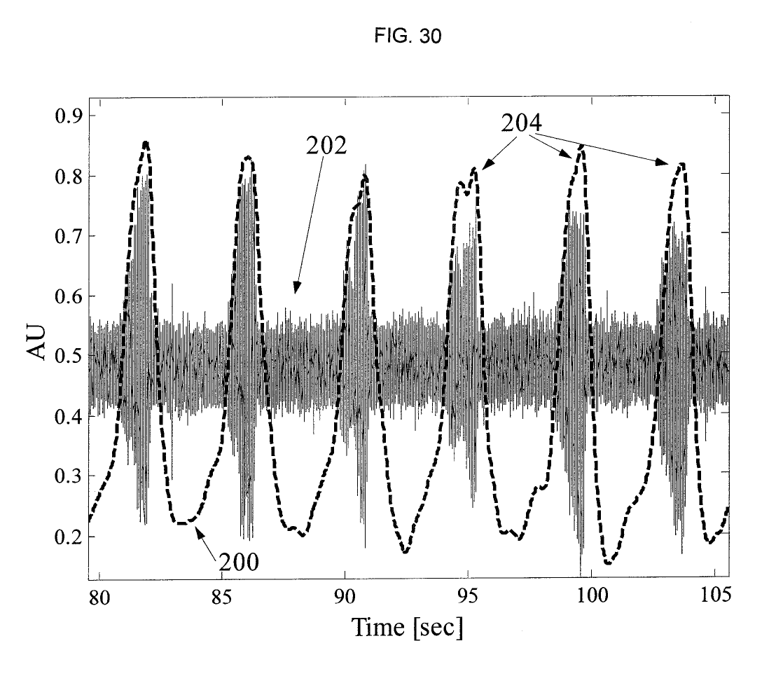

In one embodiment, system 10 monitors breathing patterns through the mechanical channel and the acoustic or audio signals, for example, snoring, through the audio channel. Snoring is identified as a significant acoustic signal that is time correlated with the breathing pattern. The system recognizes epochs, that is, time periods, that include loud snoring. The system marks events as partial OSA when the audio signal decreases although the breathing effort remains constant or even increases. FIG. 30 shows an example of partial OSA as recorded by the system, obtained from an 8-year-old asthmatic patient during the night. Line 200 in FIG. 30 denotes the breathing pattern and line 202 denotes the associated audio signal. The breathing efforts in the last 3 cycles, 204, are similar to the efforts in the first 3 cycles, whereas the audio amplitude in the last 3 cycles, 204, are significantly decreased compared to the audio amplitude during the first 3 cycles. In one embodiment, system 10 also monitors the heart rate simultaneously with the above and verifies a suspicious apnea event by looking for the characteristic change in heart rate.

In one embodiment, the system monitors breathing patterns through the mechanical channel and snoring through the audio signal. The system recognizes increasing respiratory motion with decreasing audio signal leading up to a restlessness event. The system identifies this pattern as a probable OSA pattern.

In one embodiment of the present invention, the system identifies the recurring pattern of OSA or CSA for the subject and identifies the pattern that precedes the apnea event, for example, the gradually decreasing amplitude of the respiration motion before CSA in a patient suffering from Cheyne Stokes Respiration (CSR) or the initial labored breathing with reduced audio signal of OSA or the deep inspiration before CSA. Upon identifying the pattern that precedes the apnea event, system 10 immediately activates a therapeutic device to prevent the apnea event from taking its full course. The therapeutic device can be, for example, a Continuous Positive Airway Pressure (CPAP) system which is placed on the patients face continuously but only activated on an as needed basis. Once the respiration pattern returns to normal, or the apnea at least subsides, and the therapeutic device is no longer needed, system 10 turns off the therapeutic device until the next oncoming apnea event is identified. In such a way the system prevents apnea events while not having to constantly operate the therapeutic device which may make falling asleep more difficult or have other side effects.

In one embodiment, system 10 monitors respiratory rate and identifies respiratory depression as a significant decrease in respiration rate compared to baseline. Upon detection of respiratory depression the system indicates that information and in some cases activates an alarm through user interface module 24. The system is useful, for example, for monitoring post operative patients as well as patients who have been treated with opioids, barbiturates, etc. In some instances, the use of such a monitoring system to detect and alarm upon a respiratory depression enables the clinician to use such drugs where otherwise they would not be used. In other cases, it enables the clinician to increase the dosage of these drugs.

In one embodiment, system 10 detects changes in respiration rate, heart rate, and body motion that indicate that the patient is suffering from pain. In one embodiment, the system activates, upon detection of pain, drug administration device 266 in order to alleviate the pain automatically with predefined dosage of the appropriate medication.

In one embodiment, motion sensor 30 is implemented as an accelerometer that is mounted on the body of subject 12, implanted in the body, or in a contact-less manner under the mattress, mattress pad, mattress cover, or in the pillow.

In one embodiment, the motion sensor 30 provides a 3 dimensional motion signal (e.g. a 3 dimensional accelerometer). Such a breakdown into axes enables improved separation between mechanical signals resulting from respiratory motion and from the heart beat. The signal resulting from heart beat (cardio-ballistic effect) is generally strongest in the axis that is parallel to the length of the body from head to toe while the respiratory signal is strongest in the axis that is parallel to depth of the body from the backbone to the chest.

In the treatment of premature ejaculation, it is necessary to have a monitor for the length and frequency of sexual intercourse. In one embodiment system 10 is used to monitor sexual intercourse. The motion sensor detects the rhythmic motion of sexual intercourse. Pattern analysis module 16 identifies the characteristic frequencies of motion indicative of sexual intercourse and may in addition analyze characteristic audio signals indicative of sexual intercourse. The system logs the duration and frequency of sexual intercourse.

In one embodiment, motion sensor 30 is implemented as a piezo-electric sensor. In one embodiment, motion sensor 30 is implemented in a mechanical structure that is designed to resonate at a frequency that is close to the frequency of the heart rate in order to maximize the sensitivity of the sensor to the pulse measurement.

In one embodiment, motion sensor 30 is placed in a pillow or in the vicinity of the head of subject 12 while he sleeps in order to identify teeth gritting.

In one embodiment, system 10 monitors respiration pattern, heart rate pattern and detects changes in pattern that precede changes in blood oxygen level. The system then serves as an early warning system for change in blood oxygen level. In some cases the changes in heart beat pattern and respiration rate and respiration motion pattern precede the changes in blood oxygen level. System 10 has blood oxygen level meter and learns the characteristic changes in heart beat pattern, respiration rate pattern and respiration motion pattern that precede the change in blood oxygen level for the subject 12. Upon detecting these learned patterns the system then provides an earlier warning of a change in blood oxygen than is possible with just the blood oxygen level meter.

In one embodiment the system 10 is installed in an automobile with the sensor installed in the driver's seat. System 10 monitors the driver's respiratory, heart and motion pattern to identify signs that indicate that the driver is falling asleep or otherwise losing his capacity to drive the car (intoxication, heart attack, etc.). In one embodiment system 10 is installed in a chair in which the patient is used to sitting at home or at work.

In one embodiment, system 10 is installed in a wheel chair and performs continuous monitoring of subject 12 while he/she sits in the wheel chair. In one embodiment, system 10 includes one sensor in a wheel chair and one sensor in the bed. The data from both sensors is relayed to a single pattern analysis module 16 using wired or wireless communication. This enables system 10 to have a more extensive monitoring of the patient throughout the daily routine. In one embodiment, system 10 is implemented as a watch worn on the hand of subject 12.

In one embodiment, system 10 is used to analyze the respiration and heart rate pattern of a Congestive Heart Failure (CHF) patient and to identify the change in pattern characteristic of pulmonary edema. In one embodiment, system 10 identifies the change in the cardio-ballistic effect measured in the vicinity of the subject's legs which is indicative of edema in the legs. In some cases, patients who enter the bed with edema at the beginning of the night have the fluids move to the area of the abdomen while they lie horizontally during the night. System 10 identifies the change in these parameters along the night and provides an estimated measure of the level of edema and the level of change.

In one embodiment, pattern analysis module 16 is adapted to identify preterm labor in a pregnant woman. Preterm labor is the leading cause of perinatal morbidity and mortality in the United States. Early diagnosis of preterm labor enables effective tocolytic therapy to prevent full labor. In one embodiment, system 10 is adapted to identify the mechanical signal of contractions. In one embodiment, motion sensor 30 is adapted to include multiple sensors located in the vicinity of the legs, pelvis, lower abdomen, and upper abdomen. Pattern analysis module 16 identifies a mechanical signal that is strongest in the area of the lower abdomen and pelvis and weaker in the upper abdomen as a signal indicative of contractions. In one embodiment, system 16 is adapted to differentiate between Braxton Hicks contractions and normal contractions in order to minimize false alarms of preterm labor. In one embodiment, differentiation between regular contractions and Braxton Hicks contractions is done by comparing the frequency and strength of the contractions. In one embodiment, the strength of the contraction mechanical signal is normalized by the strength of the rhythmic heart and respiration signals. In one embodiment, the system logs the contractions and alerts the subject or a clinician upon having the number or hourly rate of contractions exceed a predefined threshold.

In one embodiment, system 10 is installed within a bed mattress. The display is either integrated into the mattress as well or projected from the mattress onto the wall or ceiling. In one embodiment, the data displayed or projected is used for the purpose of biofeedback in order to help the subject reduce respiration rate and heart rate as a treatment for stress. In one embodiment, the embedded system includes also a weight sensor. This is used both for the identification of CHF deterioration as well as for calculation of drug dosage per weight.

In the analysis of the heart rate signal, in some cases it is useful to minimize the respiration related signal. In one embodiment, the pattern analysis module 16 analyzes the breathing related signal and identifies the time segments when there is no respiration related movement—in most cases there is such a brief period as part of every breathing cycle. During that brief period the system identifies the heart rate related signal and analyzes it effectively with minimal interference from the respiration signal.

In one embodiment there are several mechanical sensors, such as, weight sensors, may be distributed along the mattress. The system calculates the subject's weight distribution between the different sensors. If the subject is suffering from edema a larger portion of his weight is expected to be found in the area of the legs which enables detection of the edema. In another embodiment, the system detects the change in weight distribution along the night. If the subject is suffering from edema, the fluids are expected to move from the area of the legs to the upper torso due to gravity and this change in weight distribution is used as an indication for the existence of edema. In one embodiment, the plurality of sensors is implemented in an air mattress placed above, below, or instead of the standard bed mattress. The air mattress is divided into compartments—each compartment has a separate pressure sensor. The pressure measured by the sensor in each compartment is indicative of the weight of the patient's body in that area of the bed. The mechanical sensors may be pressure sensors; vibration sensors; strain sensors, such as, strain gauges; accelerometers; or any sensor adapted to detect a motion or load.

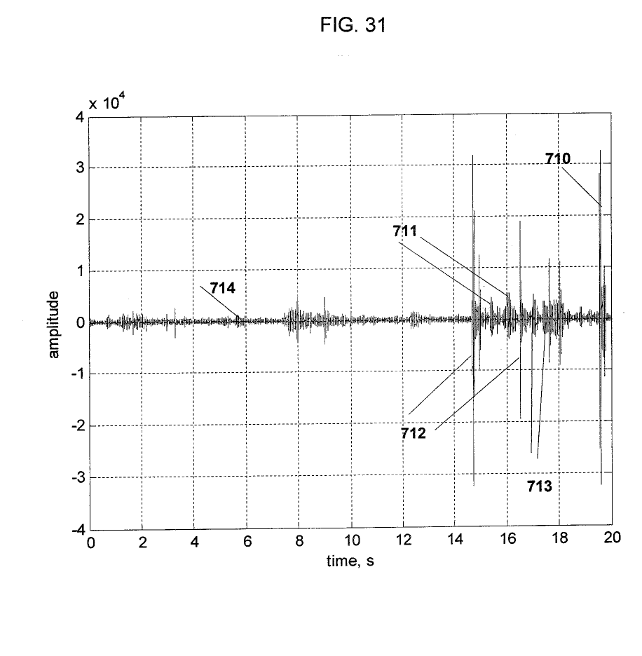

In one embodiment of the present invention, system 10 provides cough monitoring. In that embodiment, system 10 measures the number of cough events during the monitoring period and the time of each cough occurrence. In one embodiment, system 10 detects cough using acoustic recording of the ambient audio signal in the vicinity of subject 12, for example, by sensing an audio signal near the subject, such as by placing a microphone within 50 cm of the subject. The system digitally analyzes the signal recorded from the acoustic sensor which is part of system 10 and identifies acoustical events that are larger than the background noise level. System 10 distinguishes between cough and non-cough acoustical events. The latter may be human generated speech, laughing, sneezing or snore, mechanical high amplitude impulse-like noise, TV, radio, etc. FIG. 31 shows an example of the recorded segment with different acoustic events: cough 710, speech 711, mechanical high amplitude impulse-like noise 712, and mechanical “murmur” 713 all much higher than general noise level 714.

In one embodiment, the time intervals that include acoustical events are selected using signal energy and amplitude thresholds. Thresholds are calculated per constant length segment of the acoustical record that includes a number of events and noise intervals. The segment is divided to frames of fixed small length. In one embodiment the frames do not overlap. In another embodiment the frames with overlapping are used. For each frame signal energy and maximum amplitude are calculated and corresponding distributions of their values are obtained. Thresholds are extracted from these distributions following usual tail considerations. Frames for which the values calculated are higher than the thresholds are united in intervals with acoustical events. Very short and too long intervals and intervals with small number of amplitudes over threshold are rejected.

In one embodiment, in order to detect a cough the system first rejects signals that are identified as vocal or that have a length that is shorter or longer than thresholds and then examines the specific frequency change pattern that is indicative of a cough.

Relevant background material about the three-phase cough structure is available in: “Towards a quantitative description of asthmatic cough sounds.” C. W. Thorpe, L. J. Toop, K. P. Dawson. Eur. Respir. J, 1992, 5, 685-692. The cough is considered as a sequence of the initial glottal opening burst—phase 1, the quieter middle phase—phase 2, and (sometimes) the final closing burst—phase 3.

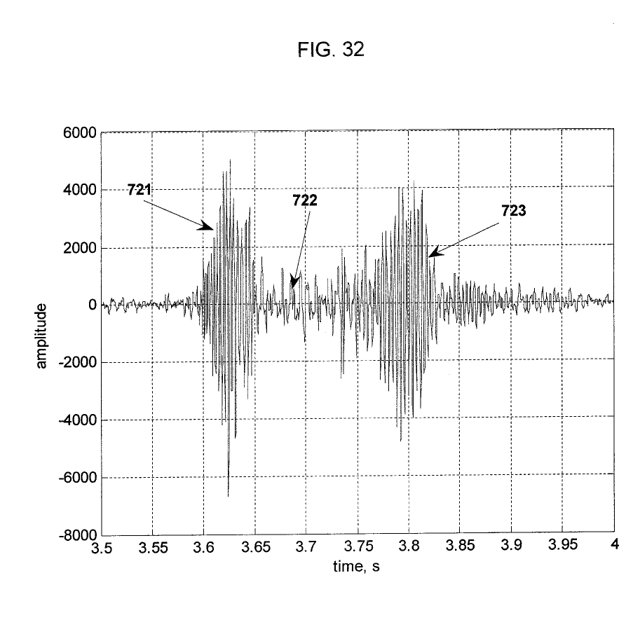

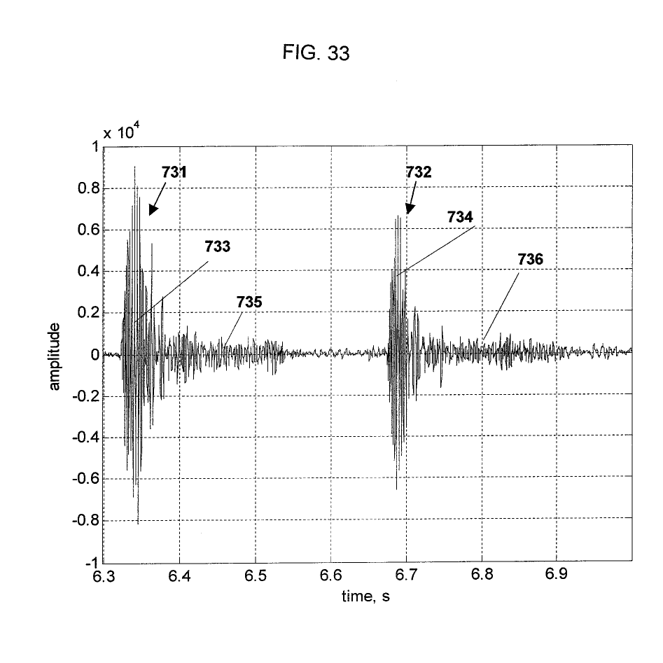

FIG. 32 shows an example of the 3-phase cough: phase 1—short initial burst 721, phase 2—722 and phase 3—723. FIG. 33 shows an example of the two sequential 2-phase coughs 731 and 732—both coughs without phase 3. First phases 733 and 734 are short, about 0.04-0.05 seconds (secs.) in duration. Duration of second phases 735, 736 is about 0.17 secs.

In one embodiment, system 10 uses only phase 1 in order to identify the cough. System 10 recognizes the pattern of phase 1 using spectral estimation based on the Autoregressive (AR) method. An AR model is calculated per sliding window that moves over the time interval including the acoustical event. The AR model is then analyzed to calculate the power spectral distribution (PSD) over the window. Frequencies that correspond to maxima points of PSD (there may be more than one) are taken as characteristic frequencies for that time window. By attributing to each maxima point the start time of the window, one gets the time-frequency characteristic(s) of the time interval.

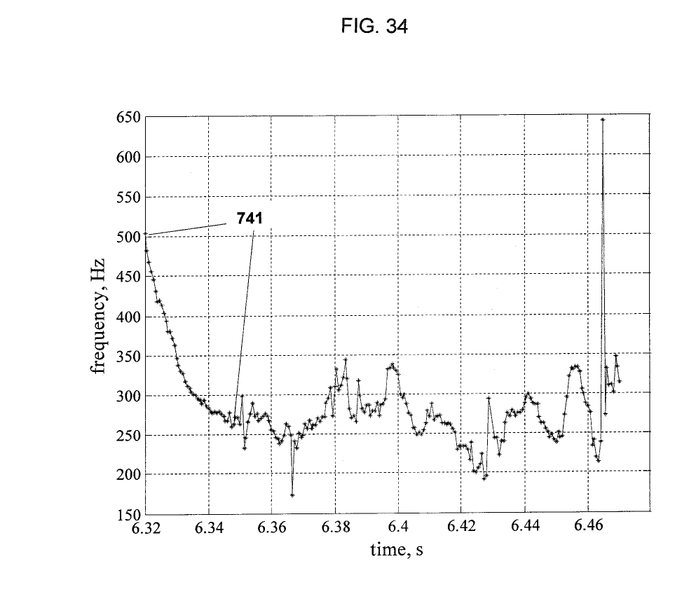

In one embodiment, phase 1 of the cough is identified by looking for a significant decrease of time-frequency characteristic over a significant part of the time interval's duration. FIG. 34 is a graph illustrating the behavior of AR time-frequency characteristic over an interval that includes cough phases 1 and 2. It corresponds to the first cough 731 on FIG. 33. The duration of phase1 is about 0.04 secs. It corresponds to signal in the interval about 6.32-6.36 secs. Significant frequency decrease 741 takes place over interval 6.32-6.35 secs. This enables the system to detect phase 1 and accordingly identify the cough and its time.

In one embodiment, the length and shifting of the sliding window should satisfy two conditions:
1. The length must be long to include enough sampling points for AR model calculation
2. The length and the shift must be short to get the representative number of points in the time-frequency characteristic.

In one embodiment the order of the AR model is a predefined constant. In another embodiment the order of the AR model is calculated using Minimum Descriptive Length algorithm or any similar algorithm.

In one embodiment only one highest maximum frequency per sliding window is taken for analysis. In another embodiment two maxima frequencies per sliding window are taken for analysis.

In one embodiment, an additional or alternative characteristic of the acoustical signal used to identify cough is the envelope of the acoustical signal in the time domain. The envelope is calculated as a set of points representing standard deviation per moving window with proper scaling and smoothing. In one embodiment, standard filtering like non-linear weighted least mean square is used. The form of cough event envelope depends on presence of phase 3. If only phases 1 and 2 are present then the envelope has specific geometry with single maximum. If all three phases are present then the envelope has two-hump form. In one embodiment, the system uses the envelope analysis to identify coughs and to differentiate between coughs with phase 3 and coughs without phase 3. In one embodiment, the data regarding coughs with and without phase 3 is displayed to a patient, clinician or used by system 10 as a clinical parameter data for determining the condition of the patient and any change compared to baseline.

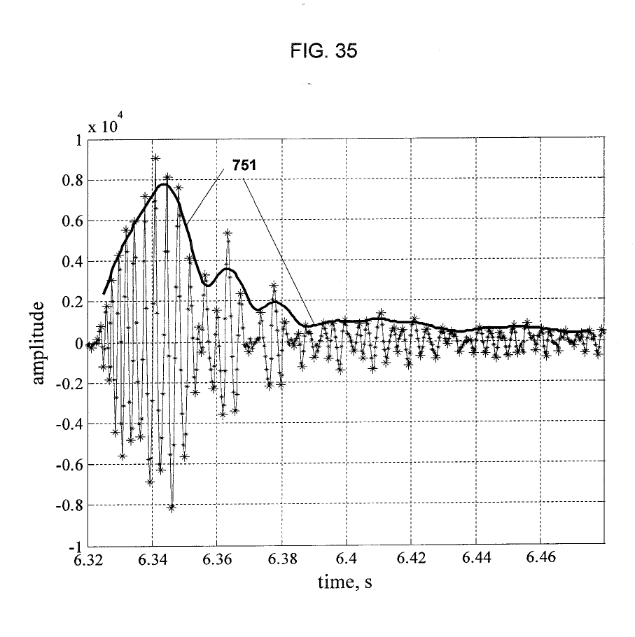

In one embodiment the cough envelope detection is based on calculation of the number and location of intersection points between the above mentioned envelope and least mean square polynomial estimation of that envelope. In another embodiment a Dynamic Time Warping algorithm is applied to test the envelope. FIG. 35 presents the envelope 751 of the same cough event as at FIG. 33 (738) and FIG. 34.

In one embodiment, specific patterns that characterize non-cough acoustical events are calculated using frequencies related to signal amplitude zero-crossing points and time-frequency AR characteristic(s) calculated as described above. In one embodiment, the pattern that distinguishes the vocal, i.e., non-cough acoustical event from cough events is the concentration of frequencies around small number of fixed values. If this pattern is identified using either zero-crossing and/or AR methods then the event is considered as vocal and not a cough.

In one embodiment, zero-crossing frequency calculation is replaced by maximum/minimum detection. In one embodiment, a combination maximum, minimum and zero-crossing analysis is used in order to smooth the resulting frequency distribution.

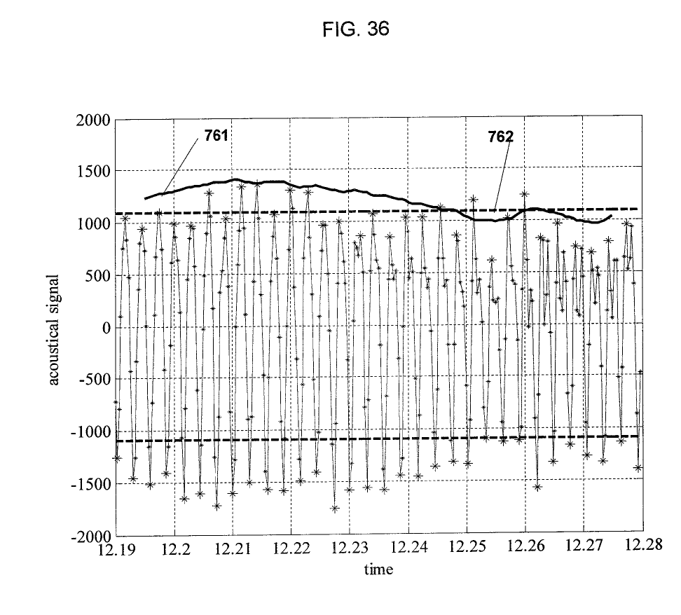

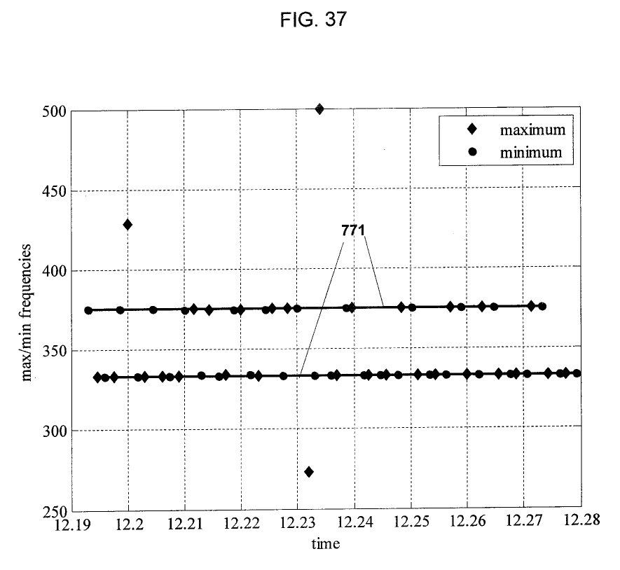

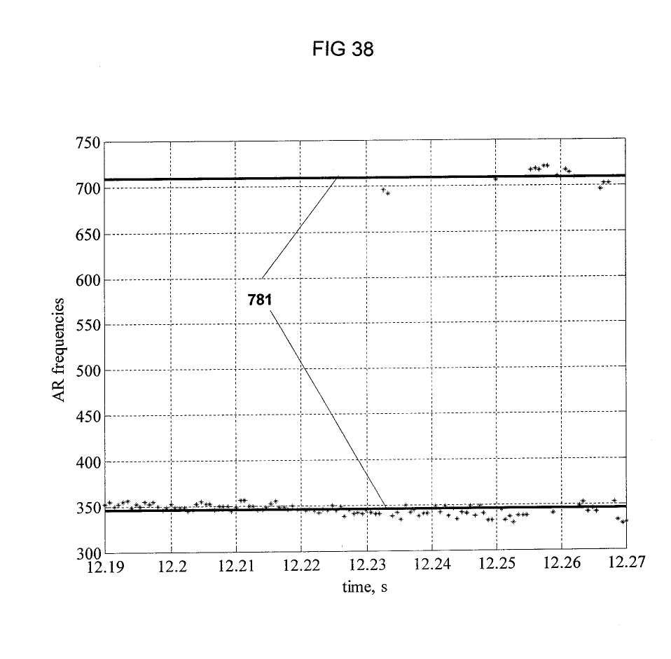

FIGS. 36, 37, and 38 show an example of vocal acoustical event and its patterns as measured by an embodiment of the present invention. FIG. 36 presents the recorded signal, its envelope 761 and amplitude threshold 762. FIG. 37 presents the distribution of maximum/minimum frequencies. Localization of frequencies (except 3 points) around 2 values 771 shows the vocal pattern. In some instances, the frequencies may be distributed around a larger number of values. FIG. 38 shows the distribution of AR frequencies. Localization of AR frequencies around 2 values shows the vocal pattern.

In one embodiment, cough is detected using a combination of an acoustical signal measured by acoustic sensor 110 (see FIG. 2) and a mechanical motion signal measured by motion sensor 30. The mechanical signal not associated with cough may include among others the following:
1. Breathing motion, i.e., a periodic signal with 1-6 sec period, and heart beat vibration with a 0.3-2 second period;
2. Non-stationary dynamics due to body restlessness with time constant of about 1 sec.;
3. Transitive processes associated with the sensor with a time constant of about 10 seconds; and/or
4. External mechanical impulse.

For the purposes of this disclosure, mechanical dynamics is called slow over a specific interval if the signal may be approximated by an exponent with time constant greater than 1 second. A quiet mechanical event is defined as one having a time interval when mechanical signal represents breathing, heartbeat, or slow dynamics.

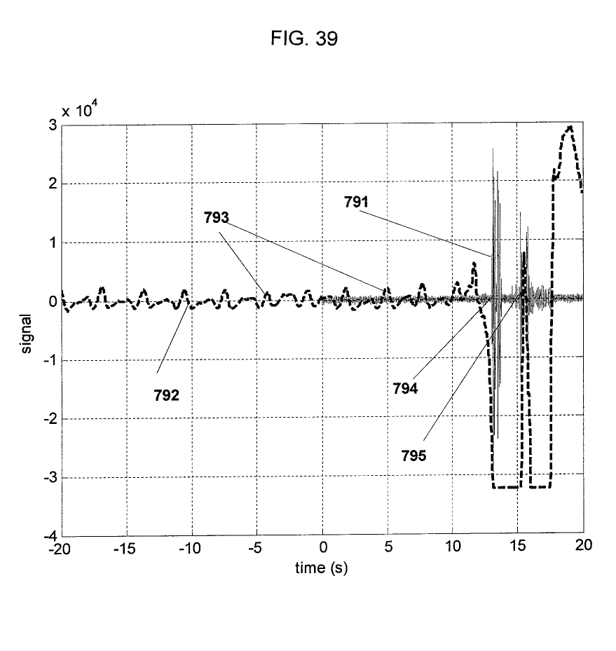

In one embodiment, cough analysis module 26 of system 10 marks or identifies a cough when the appropriate acoustical signal is accompanied by a simultaneous strong and fast body motion signal compared to that of a normal motion signal, for example, only due to respiratory motion. For example, in one embodiment, module 26 continuously calculates the first derivative of the respiratory motion signal and sets a criterion, for example, of at least 3 times the level of that first derivative of the respiration signal, for example, the relatively steady-state motion signal before the cough episode (as indicated, for example, by 793 in FIG. 39). A combined motion/acoustic event is marked as a cough if, in addition to the acoustic criteria discussed above, the first derivative of the motion signal exceeds that of the criterion at the same time. In some embodiments, an exception to the rule may be allowed in cases when the mechanical sensor signal reaches saturation level.

FIG. 39 shows an example of the cough pattern mechanical signal as measured by an embodiment of the present invention—that is, a significant amplitude change due to body movement induced by cough. In FIG. 39, there are presented simultaneously sound or audio signal 791 and mechanical motion sensor signal 792. The mechanical signal 792 is presented for the same time segment as the audio signal 791 and for a previous time segment. The cough episode is shown as the increase in amplitude of audio signal 791 identified at 794. Before the cough episode 794, the mechanical signal 792 represents breathing pattern 793. In the close vicinity of the cough episode 794, initial burst (phase 1) takes place with a large amplitude and very fast mechanical movement perturbation (significant decrease in mechanical signal 792). There is the same pattern—that is, a significant change (increase) of the mechanical signal—near the phase 1 related to the second cough episode 795.

In one embodiment the system detects an acoustic signature for the cough that is different for cough with fluids in the lungs (pulmonary edema) and for cough without fluids in the lungs (normal condition). This enables earlier warning for deterioration of congestive heart failure deteriorations. In one embodiment the system detects a cough signature that is different for a smoking person as compared to a non smoking person.

In one embodiment, system 10 includes at least 2 acoustic sensors. One sensor is placed under the mattress or sheet and the other is placed, for example, at the bedside. Correlation of the at least two sensors allows improved identification of the source of the sound. For example, sound that is received only by the sensor placed under the mattress is interpreted as being caused by a mechanical source in the bed, e.g., a hand hitting the mattress. Sound that is received by the external acoustic sensor but not by the sensor in the bed may be caused by a source outside the bed.

Sleep Disturbances

Sleep disturbances have been associated with asthma in children and adult patients. Restless sleep has been reported in more than 80% of adult asthmatic patients and in 61% of the asthmatic children (see (a) Fitzpatrick, M. F., et al., “Snoring, asthma and sleep disturbances in Britain: A community based survey,” Eur. Respiratory J 1993;6:531-5; (b) Jobanputra P., et al., “Management of acute asthma attacks in general practice,” Br J Gen Pract 1991;41:410-3; (c) Lim T. O., et al., “Morbidity associated with asthma and audit of asthma treatment in outpatient clinics,” Singapore Med J 1992;33:174-6; and (d) Madge, P. J., et al., “Home nebuliser use in children with asthma in two Scottish Health Board Areas,” Scott Med J 1995:40:141-3, which are all incorporated herein by reference).

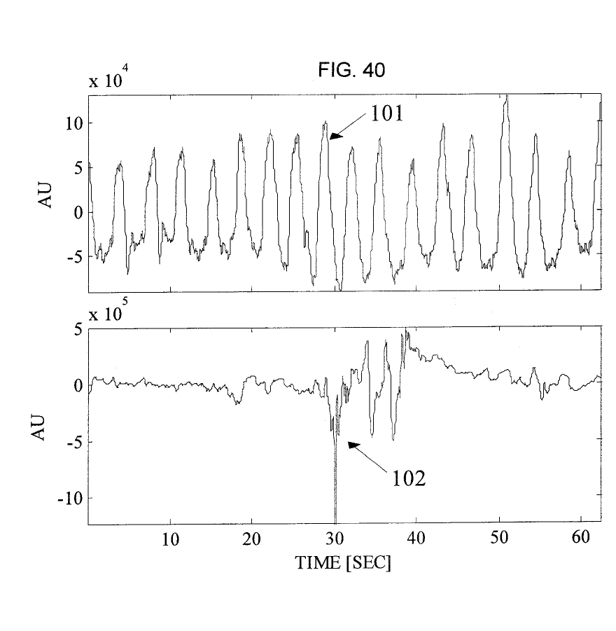

In one embodiment, system 10 distinguishes between quiet sleep and sleep disturbances. During quiet sleep, the system measures periodic motion of the body related to respiration or heartbeat, whereas during restless periods the system senses mainly the sudden body motion. FIG. 40 shows an example of quiet sleep (line 101) and a restless event (line 102) as measured by an embodiment of the present invention. In this disclosure, “quiet sleep” is considered to be any time period in which the subject lies quietly on the bed and a cyclical respiratory signal is detected, even though the subject may actually be awake.

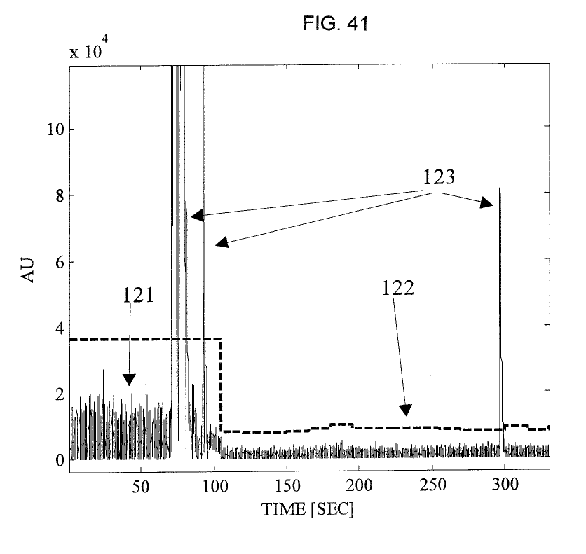

In one embodiment, in order to detect restless events, a threshold level is defined according to the amplitude of the signal during quiet sleep. For example, system 10 detects an epoch with periodic respiratory motion and defines the threshold as 5 times the standard deviation of the signal in that time epoch. The threshold remains constant until a new epoch with similar characteristics is detected. FIG. 41 shows an example of the data signal acquired by an embodiment of the present invention (absolute value shown as line 121) and the threshold level defined by the algorithm described above (line 122). Note that the threshold level is not affected by the sleep disturbances (peaks 123).

In one embodiment, several parameters are defined in order to evaluate the quality of sleep:
1) Total time of restless sleep—the cumulative time that the data signal is above the threshold.
2) The total power of disturbances—the area (integral) of the data above the threshold.
3) Sleep efficiency—the ratio between epochs with quiet sleep and the total sleep epochs.

In one embodiment, system 10 additionally detects arousal events according to the duration of each restless event. For example, a restless event that lasts longer than 15 seconds is defined as an arousal.

In one embodiment, system 10 adds the above defined restlessness values to the clinical parameters as defined herein above, and defines a baseline and a clinical score which includes these parameters.

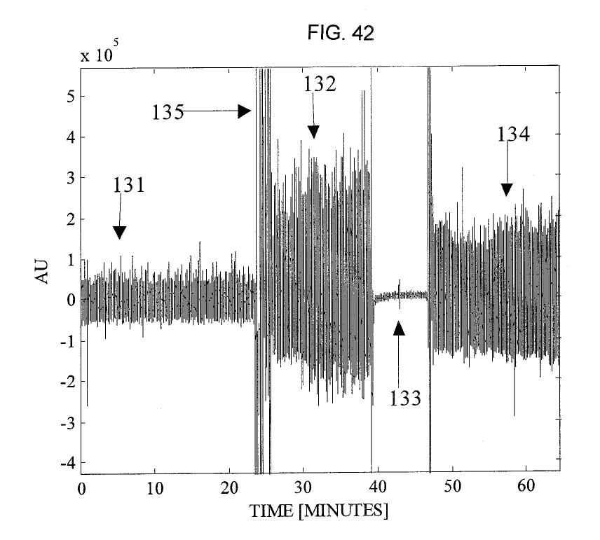

Another parameter related to the quality of the sleep is the number of changes in sleep posture. In one embodiment, system 10 detects a change in sleep posture according to the amplitude of respiratory induced signal. FIG. 42 shows an example of three changes in sleep posture that occurred during a period of 25 minutes as measured with respect to a human patient, in accordance with an embodiment of the present invention. Areas 131, 132, 133, and 134 show four different sleep postures as indicated by the significant change in signal amplitude. Note that in this case each change in posture is accompanied by a restless event (peaks 135, for example).

Heart Rate

In one embodiment, system 10 is adapted to sense respiration motion as well as heart beat. In one embodiment, pattern analysis module 16 differentiates between respiration and heart beat signals using band pass filters with appropriate cutoff frequencies. For example, a filter of 1-1.5 Hz (corresponding to 60-90 BPM) can be used for patients with expected heart rate range of 70-80 BPM. After filtering, the device calculates a Fourier transform for each epoch and the main spectral peak is considered to represent the heart rate.

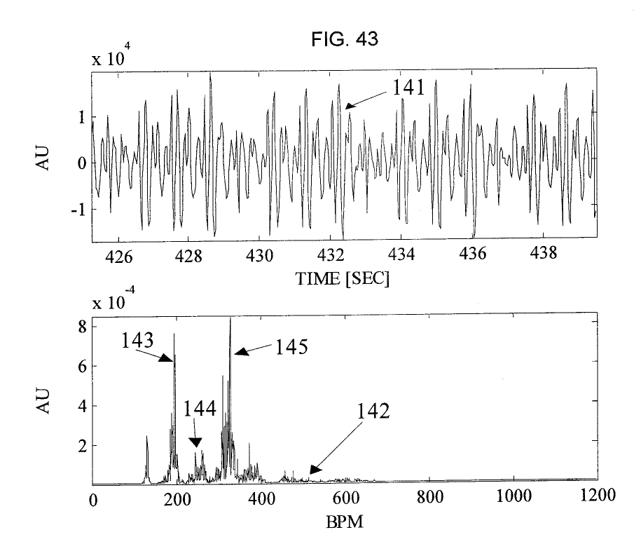

In some cases, especially when the heart rate is relatively low, higher harmonics of the respiration rate may appear in the spectrum of the heart channel and may affect the measurement of the heart rate. In one embodiment, system 10 uses a band pass filter which eliminates most of the respiratory harmonics (as well as the basic frequency of the heart rate), using, for example, a pass band of 2-10 Hz. In a Fourier analysis of the resulting signal, the basic frequency of the heart rate is no longer the highest peak. However, the harmonics of the heart rate signal are still present. Heart beat pattern analysis module 23 identifies these peaks and calculates the heart rate by calculating the distance between consecutive peaks. FIG. 43 shows an example of the time series calculated in one example using the above-defined filter (line 141) and the corresponding power spectrum (line 142). In this example, peaks 143, 144, and 145 are identified and the heart rate is calculated as the BPM difference between peak 144 and 145 or peak 143 and 144, or half the difference between peak 145 and 143. The existence of peak 144 exactly at the halfway point between peaks 143 and 145 provides verification that the distance between peaks 143 and 145 should be divided by two in order to get the correct heart rate.

In another embodiment, system 10 calculates the heart rate using an amplitude demodulation method. In this method, a band pass filter which rejects the basic heart rate frequency as well as most of the respiratory harmonics is used. For example, the band pass filter may be tuned to 2-10 Hz. The absolute value of the filtered signal is calculated, and a low pass filter with appropriate cutoff frequency (e.g., 3 Hz) is applied to the absolute value signal result. Finally, the power spectrum is calculated and its main peak, which corresponds to the heart rate, is identified.

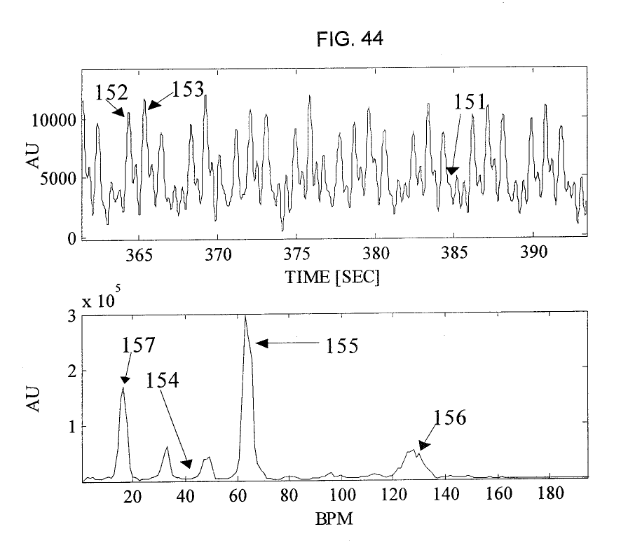

FIG. 44 shows results of such analysis performed by an embodiment of the present invention. Line 151 indicates the demodulated measured time series following the above band pass filter. Arrows 152 and 153 point to successive heart beat cycles. Line 154 shows the corresponding power spectrum of the absolute value of the time series and peak 155 indicates its main peak, which reflects the heart rate. In addition, peak 156 indicates the second harmonic of the heart rate and peak 157 indicates the respiration rate.

Tremor

There are multiple clinical uses for the measurement of tremor. One application is the monitoring of diabetic patients to identify hypoglycemia. Typically, tremor-related oscillations exist in a frequency band of 3-18 Hz. In one embodiment, motion data acquisition module 20 and pattern analysis module 16 are adapted to digitize and analyze data at those frequencies. A significant change in the energy measured in this frequency range is attributed to a change in the level of tremor, and the change in the spectrum of the signal is attributed to a change in the spectrum of the tremor.

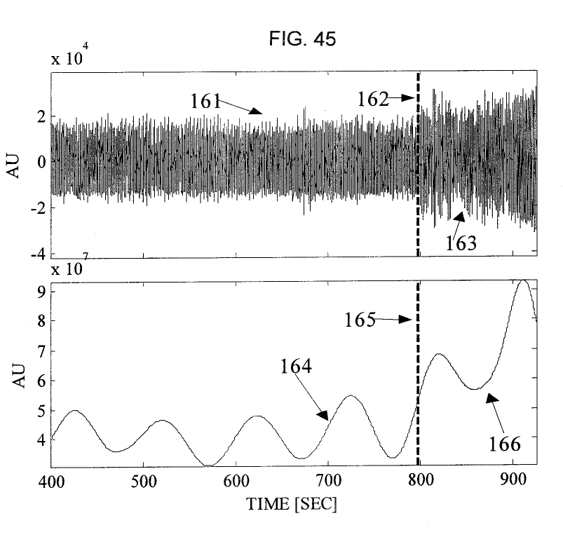

FIG. 45 shows an example of data acquired and analyzed by one embodiment of the present invention in monitoring a human subject with voluntarily induced increased tremor. The top graph shows the sampled data filtered with a band pass filter at 2-10 Hz (line 161) as a function of time. The dashed line 162 indicates the timing where the voluntarily induced increased tremor began. Area 163 (on the right side of line 162) shows the effect of the increased tremor, which caused an increase in signal amplitude. The bottom graph shows the corresponding time dependent total spectrum power at the frequency band of 3-9 Hz (line 164). Line 165 indicates the timing where the stimulated increased tremor began. Area 166 (on the right side of line 165) shows the increased tremor energy measured by that embodiment.

In one embodiment, system 10 first identifies the signal associated with heart rate and respiration rate. The system subtracts the heart rate and respiration rate signal from the overall signal. The resulting signal in those areas where there are no restlessness events is regarded as the tremor signal for the above analysis. In one embodiment, the energy of the tremor signal is normalized by the size of the respiration and/or heart signal.

Sleep Stages

REM (Rapid Eye Movement) sleep is characterized by periodic eyelid fluttering, muscle paralysis, and irregular breathing. In one embodiment, system 10 analyzes breathing pattern on a cycle-to-cycle basis in order to distinguish between REM and non-REM sleep.

In one embodiment, breathing pattern analysis module 22 calculates the breathing rate variability (BRV) for subject 12. This is done by taking the filtered breathing related signal and identifying the peaks using standard peak detection algorithms (for example, using auto-correlation methods). Every time epoch, e.g., one minute, the standard deviation of the time between respiration peaks is calculated. This is defined as “the BRV.”

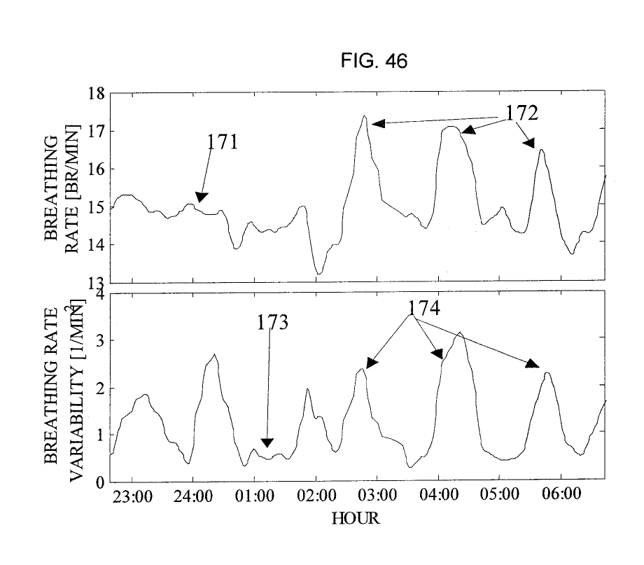

FIG. 46 shows an example of breathing pattern during a night as was recorded by one embodiment of the present invention on a human subject. Line 171 in FIG. 46 shows a 1 minute average breathing rate during the night, and line 173 shows the 1 minute breathing rate variability (BRV). High variability means irregular breathing. Peaks 172 and 174 indicate epochs, that is, time periods, in which both the average breathing rate and BRV increase. These are identified as REM periods, that is, according to aspects of the invention, peaks in the breathing rate, the BRV, or both can be used as indicators of REM sleep.

In one embodiment, the system has an “alarm clock” function programmed to wake up the subject 12 at the optimal time versus the REM sleep cycle in a similar way to the product “Sleeptracker” (manufactured by Innovative Sleep Solutions, Inc., of Atlanta, Ga., USA) but without contacting or viewing the subject's body and clothes.

In one embodiment, system 10 activates drug administration device 266 upon detection of REM sleep in order to deliver certain therapies that are most effectively administered during REM sleep. In one embodiment, system 10 activates device 266 a certain predefined time after the termination of REM sleep so as to have the drugs delivered in non-REM sleep. In one embodiment, system 10 delivers the therapy after a predefined number of sleep cycles.

In one embodiment, after system 10 identifies REM sleep, system 10 is adapted to identify changes in respiratory pattern that may indicate deterioration of the respiratory condition during that time period, for example, as an early indication of the subject's chronic condition. For example, the respiration rate may increase more dramatically during REM when the asthma condition is deteriorating as compared to when there chronic condition is stable. For example, asthma and COPD patients are expected to have more difficulty breathing during REM sleep because there is less use of auxiliary muscles during REM. This enables earlier identification of deterioration and early warning enabling intervention.

Breathing Rate Pattern

Lung function is usually highest at 4 PM and lowest at 4 AM. As a result, in general, asthma symptoms are most prevalent during the last hours of the night. Normally, asthma symptoms develop on a time scale of few days. However, in some cases a sudden exacerbation occurs at night, in which case the symptoms develop during the night.

In one embodiment, system 10 measures relevant clinical parameters continuously during the night and calculates the proportional changes in the clinical parameters at the last hours of the night compared to the minimum or optimum level during that same night. Alternatively, in one embodiment, system 10 compares the value at the end of the night compared to the value at the beginning or at an earlier point in the night. For example, in one embodiment, system 10 calculates the ratio between the average breathing rate at the last hour of sleep and the average breathing rate at the first hour of sleep. A significant increase in the ratio compared to baseline is indicated to the subject or healthcare professional as a warning sign of an oncoming asthma exacerbation. Alternatively, in one embodiment, this ratio is integrated as part of the clinical score calculated by the system.

In one embodiment, the system identifies a sudden exacerbation during the night by identifying the trend of increase in respiration rate during the night and activates an alarm to enable timely intervention to prevent deterioration of the chronic condition. In one embodiment, the system identifies a sudden exacerbation during the night by identifying the trend of deterioration in one or more of the clinical parameters during the night and activates an alarm to enable timely intervention to prevent deterioration of the chronic condition.

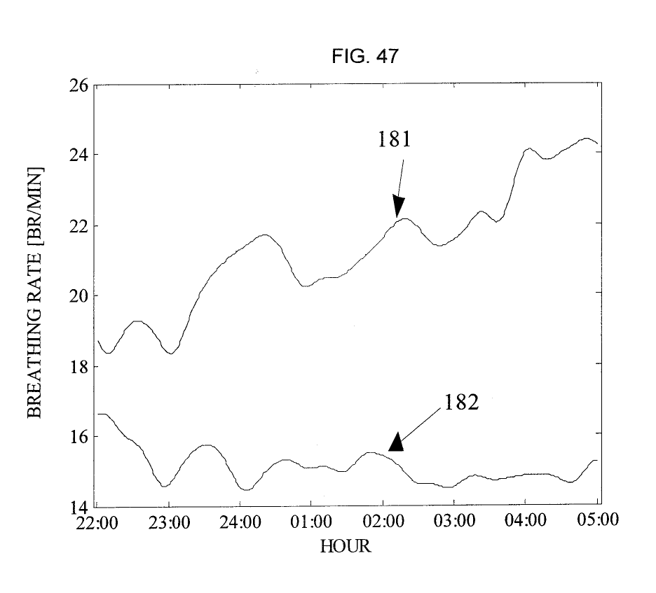

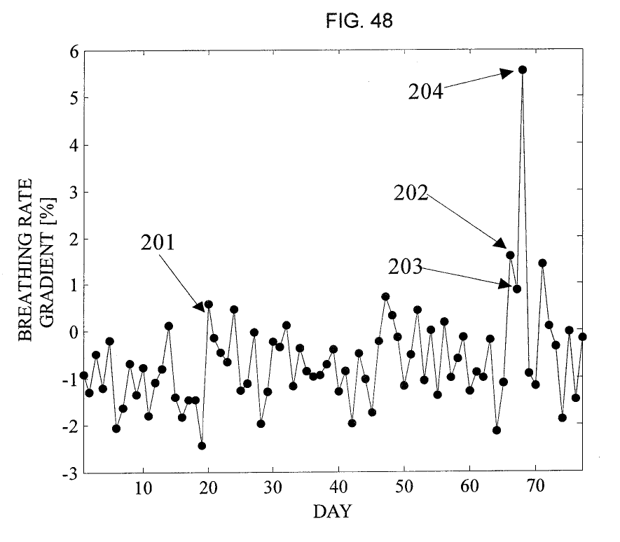

FIG. 47 shows an example of results measured by an embodiment of this invention on an asthma patient. Line 181 shows the breathing rate pattern during a night of an asthma exacerbation and line 182 shows the breathing rate during a normal night. The gradual increase in breathing rate during an exacerbation is clearly seen. FIG. 48 shows the results of an analysis by an embodiment of this invention on the data collected on an asthma patient. For each night the ratio of the average respiration rate at the last half hour of sleep to the average respiration at the first half hour of sleep was calculated. Time series 201 shows the results for a monitoring period of close to three months. Points 202, 203, and 204 correspond to a deterioration in the asthma condition as evaluated by a physician on the day between 203 and 204. In one embodiment, the values shown in FIG. 48 are integrated into the calculation of the asthma score by system 10.

Chronic patients may have limitations on intensity of physical activity in which they can engage, depending on their chronic condition status prior to beginning of exercise. Moreover, many chronic patients are prone to developing disease episodes during or after physical activity. For example, some asthma patients are prone to “exercise induced asthma.” In an embodiment, preventive treatment in response to detection of a likelihood of oncoming asthma exacerbation may be used to prevent or minimize worsening of chronic conditions due to physical activity. In asthma, for example, this is done mainly by using bronchodilators.

In one embodiment, system 10 evaluates the clinical condition of a chronic patient and determines a score for the chronic condition and accordingly displays consequent limitations, if any, on physical activity of the subject. For example, in one embodiment, the system ranks the restrictions on physical activity using a scale of breaths per minute, limiting the maximum allowed breathing frequency during exercise, based on the subject's asthma score. In an alternative embodiment, the system restricts both breathing and heart rate to maximum allowed values based on the subject's asthma score.

In another embodiment, system 10 indicates the appropriate type and dosage of preventive treatment required in order for a patient to engage in a certain degree (e.g., mild or moderate) of physical activity. For example, for asthma patients, the system may recommend usage of bronchodilators for intense short-term exercise, or a combination of bronchodilators and inhaled corticosteroids for extended exercise such as in sports tournaments.

Worsening of a chronic condition may be predicted using historical data collected and logged using trend analysis. In one embodiment, recent inter- and intra-night pattern changes in clinical parameters are compared to past data preceding previous chronic episodes. A likelihood for developing a chronic episode is derived from the degree of match of the recent clinical parameter pattern change with those of past data preceding previous chronic condition deteriorations. Alternatively, the likelihood is estimated by comparing the clinical parameter pattern with well-known patterns for that specific chronic condition.

In one embodiment, system 10 utilizes past measurements of clinical parameters to determine the likelihood of developing a clinical episode in the next day or in the next few days.

Many asthma patients are affected by environmental conditions and external irritants causing temporary or chronic worsening of their asthma status. Prediction of such worsening can be implemented by correlating current conditions with historical physiological and environmental readings known to signify an upcoming worsening of asthma status.

In one embodiment, system 10 calculates a clinical score for the subject by integrating both the clinical parameters measured for the subject as well as potential external modifiers and irritants, such as weather conditions, air pollution, and pollen count, to determine the likelihood of developing a clinical episode in the next day or in the next few days. For example, for an asthma patient, the asthma score may be increased by 10% on days of increased pollen count and then compared to a threshold to determine whether the subject or caretaker be alerted to a potential high risk condition that requires medical intervention.

PCA Analysis

Principal Component Analysis (PCA) is a mathematical way of determining a linear transformation of a sample of points in a high dimensional space which exhibits the properties of the sample most clearly along the coordinate axes. Along the new axes, the sample variances are extremes and uncorrelated.

By their definition, the principal axes will include those along which the point sample has little or no spread (minimal variance). Hence, an analysis in terms of principal components can show linear interdependence in data. A point sample of L dimensions for whose L coordinates M linear relations hold, will show only (L-M) axes along which the spread is non-zero. Thus, by using a cutoff on the spread along each axis, the dimensionality of the sample may be reduced. In practice, PCA is used to reduce the dimensionality of problems, and to transform interdependent coordinates into significant and independent ones.

In one embodiment, system 10 implements PCA analysis within pattern analysis module 16 to clinical parameter patterns recorded successively over many nights, in order to identify unique patterns signifying upcoming clinical episodes. Data are synchronized based on the time of recording during night sleep. In nights with chronic disease activity, consistent correlated patterns are identified which are significantly different from patterns of nights with no chronic disease activity. Gradual changes in the level of the chronic activity patterns are used to track worsening and improving of chronic condition. The patterns associated with chronic deterioration are either predefined within pattern analysis module 16 or are learned for the specific subject over the first (and ongoing) chronic deteriorations monitored for that subject. In one embodiment, system 10 implements the above mentioned PCA analysis within pattern analysis module 16 to clinical parameter patterns recorded successively over several nights.

In one embodiment, system 10 performs PCA analysis of clinical parameter patterns of subject 12 during nights that have been identified as non-symptomatic and creates a pattern or set of patterns that characterize those nights. The system then looks for a change compared to those patterns as an indication of the onset of a clinical episode.

In some cases, a chronic condition deterioration may start developing during night sleep, in which case the upcoming episode may be detected from analysis of the clinical parameter during that specific night. Different parameters may be used to detect pathological changes during a specific night, such as respiration rate ratios during night sleep (e.g., average ratio between second half and first half of the night) or episode-specific respiration and heart rate patterns during night sleep.

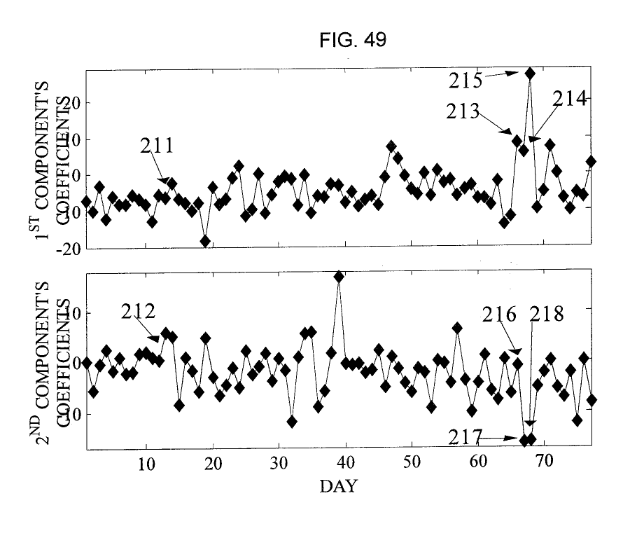

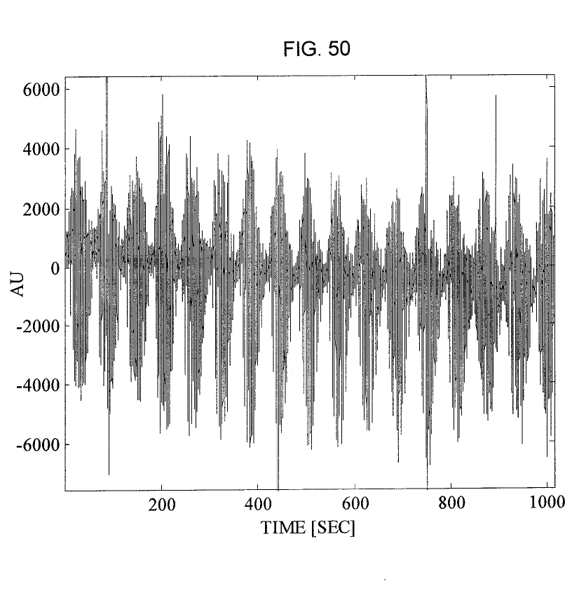

In one embodiment, the system predicts or tracks the progression of a clinical condition throughout night sleep by detection of intra-night changes in the clinical parameter patterns. Such changes may be quantified using different parameters such as respiration rate ratios at different times, or respiration rate patterns, compared to typical historical nightly behavior. In one embodiment, Principal Component Analysis is used to extract typical symptomatic and asymptomatic nightly behavior from historical readings of the patient. FIG. 49 shows the results of an embodiment of the present invention monitoring an asthma patient and running PCA on the nightly respiration rate patterns. Time series 211 and 212 show the results of the PCA analysis exhibiting the 1^{st} and 2^{nd} components respectively. Points 213, 214, and 215, respectively, correspond to an asthma exacerbation diagnosed by a physician on the day between point 214 and 215. Similarly, points 216, 217, and 218 correspond to an asthma exacerbation on the day between point 217 and 218. In a similar way, other asthma events are identified by this embodiment.

In comparing nightly patterns of clinical parameters in sleep it is sometimes necessary to shift the patterns one compared to the other based on different points in time when sleep starts and different lengths of time of the sleep cycles. In one embodiment, the system identifies the point where sleep starts and accordingly shifts each nightly pattern before conducting the PCA analysis.

In one embodiment, the system does the above shift by correlating the times of REM sleep as explained above and shifts the patterns of the clinical parameters in the optimal way so that the REM sleep times coincide and then the PCA analysis is performed.

Different chronic patients may have different responses to treatments. In one embodiment, system 10 is personalized by learning past physiological readings, past treatments, and associated past clinical scores, to provide recommendations when conditions similar to those encountered and treated in the past are re-encountered. In addition, in one embodiment, system 10 tracks habituation or adaptation processes to specific medications and accordingly adjusts the recommended dosages or suggests change of medication or combination of medications.

In one embodiment, system 10 tracks and analyzes past physiological readings, administered medication, and asthma status scores, and uses these to recommend an appropriate treatment in clinical conditions which resemble those encountered and treated in the past.

In another embodiment, system 10 monitors the effect of treatments over an extended period of time to track possible physiological habituation or adaptation to the treatment, in which case the system recommends an adjustment of the medication dosage or recommends an alternative medication or combination of medications, to maintain an adequate treatment efficacy. In one embodiment, system 10 provides an indication to the subject or physician that the current medication or dosage is losing its efficacy. For example, system 10 calculates a clinical score (e.g., an asthma score) for the patient and gets an input either manually or automatically upon the use of medication (e.g., oral corticosteroids). System 10 monitors the improvement in the clinical score upon medication use and, over multiple such events, logs the improvement in score each time a new course of medication is given. If the system identifies a clear trend of change in the level of effect of the medication on the clinical score, a notification is displayed to the subject healthcare professional or caretaker. In another embodiment, the system implements the recommended appropriate treatment by administering the required medication.

Breathing and heart rate patterns during night sleep may be used to verify that the intended asthma patient, rather than another person, is indeed being monitored by the system. The monitored physiological patterns are highly subject specific, and, during non-episodic periods, tend to vary only slightly from night to night. Towards initiation and progression of asthma episodes, a physiological trend usually builds up during several nights, enabling in one embodiment, the identification and rejection of outlier information in cases of a changed identity.

In one embodiment, the system analyzes the acquired clinical parameters to provide a warning in case of monitoring of a subject other than the intended patient. The physiological parameter values are compared to the normal parameter distributions calculated from past data of the intended patient to assess significant statistical deviations from the normal parameter distributions. Such statistical deviations are used to create a mismatch score. If the mismatch score exceeds a preset limit the system disregards the acquired data and/or provides a warning sign.

In one embodiment, the system has a central unit with a primary sensor located in the patient's bed, and secondary sensors placed in alternative sleeping sites such as a couch or different beds. The secondary sensors share data with the central unit by wire or wireless connections. In one embodiment, sensor data are validated to belong to the intended subject as described in the above embodiment, and used to create a common database for analysis.

In one embodiment, the system uses breathing patterns and accompanying acoustic sounds to identify snoring. In another embodiment, the system causes a change in the body posture in order to eliminate or reduce snoring, e.g., by changing bed or mattress angle, or increasing or decreasing head elevation by inflating or deflating a pillow.

In one embodiment, system 10 uses breathing patterns to identify sleep apnea. In another embodiment, the system attempts to restore normal breathing, e.g., by activating a continuous positive airway pressure (CPAP) device, changing bed or mattress angle, increasing or decreasing head elevation by inflating or deflating a pillow.

In one embodiment, system 10 uses respiration and accompanying acoustic sounds to identify snore and wheeze. In another embodiment, the system correlates the identified snore or wheeze with respiration cycle to indicate whether snore or wheeze occurs during inspiration or expiration.

Hypoglycemia

Hypoglycemia is usually a consequence of tight glycemic control in patients with insulin dependent diabetes mellitus (IDDM). On average, type I diabetic patients suffer from two episodes of asymptomatic hypoglycemia a week, and each year one in two patients suffers from an episode of hypoglycemia requiring the assistance of another individual (often due to seizure or coma). In addition, type I diabetic patients have a blood glucose level lower than 50 mg/dL (2.9 mmol/l) as much as 10% of the time, resulting in an untold number of pre-symptomatic hypoglycemia events.

Of special importance are the hypoglycemic episodes during night sleep. The overnight period represents the longest period of fasting of the day and nocturnal hypoglycemia may go unnoticed during sleep for prolonged periods. This is not only explained by diminished awareness while sleeping, but also by decreased epinephrine response during sleep.

In children, hypoglycemia during night sleep is a major concern. A night-time “hypoglycemia alarm” is provided to prevent this deterioration, in accordance with some embodiments of the invention. Direct continuous measurement of blood glucose level during sleep is of limited practicality with standard commercial glucose sensing products, and thus a non-invasive method for generating a hypoglycemia alarm is beneficial. Since hypoglycemia imposes an extreme metabolic deficiency, autonomic nervous system effects such as changes in heart and respiration rates, restlessness in sleep and tremor are often evident.

In one embodiment, system 10 tracks one or more critical parameters. “critical parameters,” in the context of the present patent application and in the claims, refers to respiration rate, heart rate, occurrence of palpitations, restlessness in sleep and tremor. Changes in the critical parameters associated with developing hypoglycemia during night sleep are tracked using system 10 for the purpose of providing a real-time alarm in case of an oncoming hypoglycemia episode. For example, in one embodiment, at the beginning of the night sleep, system 10 calculates the baseline reference level of one or more of the critical parameters. Then every time interval, for example, one minute, system 10 calculates the same parameters and compares them to the baseline data. A significant increase of, for example, over 25% is used as an indication of an oncoming hypoglycemia event and an alarm is activated. In one embodiment, a combined score of the critical parameters is calculated. For example, a hypoglycemia score (HypSc) may be calculated by:
HypSc=(RRS+HRS+TRS+RSS)/4 (Equation 4)
Where:
RRS=(current respiration rate)/(baseline respiration rate)*100
HRS=(current heart rate)/(baseline heart rate)*100
TRS=(current tremor level)/(baseline tremor level)*100
RRS=(current restlessness level)/(baseline respiration level)*100

Then the score is compared to a learned or predefined threshold, for example 125. If the score exceeds the threshold, an event warning is given. In one embodiment, the baseline values are the reference values at the beginning of the night sleep. In another embodiment, the baseline values are the average values measured for that subject at that same time of night during K previous asymptomatic nights, where 1<K<100, typically K=10. In another embodiment, the baseline values are population averages known for the subject's age, size, and gender.

In one embodiment, system 10 includes drug administration device 266 that delivers glucose to the patient upon detection of a hypoglycemia event. Glucose is delivered either orally or into the subject's body. In one embodiment, a drug administration device 266 dispenses a glucose spray in the vicinity of the patient's mouth to be inhaled without necessarily waking the subject and without necessarily contacting the subject's body.

Congestive Heart Failure

Congestive Heart Failure (CHF) deterioration is often characterized by abnormal fluid retention, which usually results in swelling (edema) in the feet and legs. This is often diagnosed by having patients weigh themselves daily and notice a weight increase of over 1 kg in 24 hours. However this requires patient to comply with a daily weighing routine. In one embodiment, system 10 is adapted to identify a change in weight of subject 12. In one embodiment, sensor plate 30 includes a vibration sensor which is AC coupled (i.e., includes a high pass filter, for example, at 0.05 Hz), as well as a pressure sensor which is DC coupled (i.e., no high pass filter implemented). Optionally both the vibration sensor and the pressure sensor may be implemented using a single sensing component. The amplitude of the pressure sensor's signal is proportional to the subject's weight (defined herein as the “weight signal”), but is also dependent upon the subject's location and posture with respect to the sensor. The amplitude of the heart beat related signal captured by the vibration sensor (defined herein as the “heartbeat signal”) is dependent upon the subject's posture and position as well as the strength of the cardioballistic effect. As fluids build up in the body, the subject's weight increases and the cardioballistic effect is reduced.

In one embodiment, sensor plate 30 is placed under the area of the subject's legs. In that area, the body mass increases during events of edema and therefore the cardioballistic effect will be reduced while the pressure due to body weight will be increased. Pattern analysis module 16 calculates the ratio of the weight signal and the heartbeat signal. A baseline value is calculated for that ratio. An increase in the ratio may indicate the onset of edema and is indicated to the patient or healthcare professional and/or is integrated into the clinical score calculated by system 10. In one embodiment, this signal is averaged over a significant portion of the night in order to minimize the effects of a specific body posture and/or position.

Upon the beginning of deterioration, CHF patients often choose to sleep with their heads and lungs elevated compared to the rest of their bodies. Therefore a system for detecting this elevation helps provide an early indication of CHF deterioration. In one embodiment, system 10 detects such sleep posture change. In one embodiment, multiple sensor plates 30 are placed under the mattress. A change in the elevation and angle of the top third of the body of subject 12 is identified by a change in the pressure distribution between the multiple sensors. In one embodiment, a tilt sensor is placed either on the lung area of the body of subject 12, or on the mattress or in a pillow subject 12 uses. For example, an increase in the patient's tilt angle during sleep compared to previous nights is interpreted by pattern analysis module 16 as an indication of CHF deterioration that is integrated into the subject's clinical score.

In one embodiment, sensor plate 30 is extended to cover the whole area of the mattress in order to measure the weight of subject 12. In one embodiment, sensor 30 is implemented as a flexible chamber with fluid in the chamber, for example, a liquid or gas. The flexible chamber covers substantially the whole area of the mattress and is deformed due to pressure exerted by subject 12. A pressure sensor detects the pressure in the fluid in the chamber. The pressure increases with an increase in the weight of subject 12.

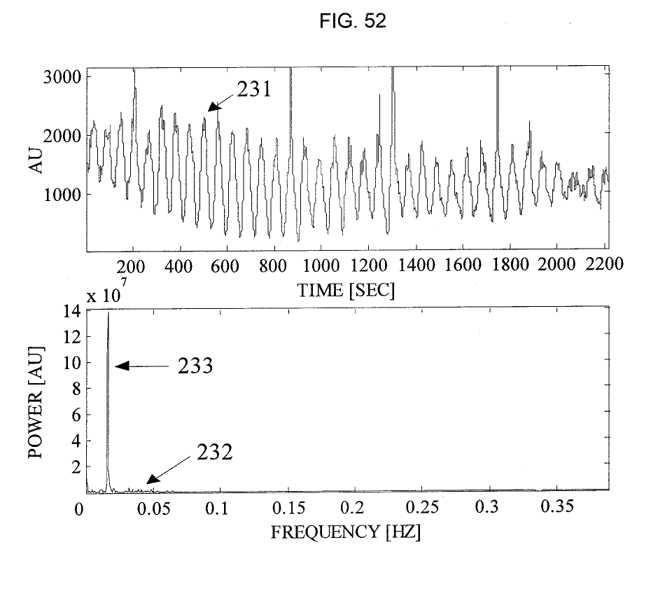

Cheyne Stokes Respiration (CSR) and Periodic Breathing (PB) are often indicators of deterioration of CHF. In one embodiment, pattern analysis module 16 is adapted to identify and measure the intensity of CSR and PB as indicators of CHF condition. FIG. 50 shows the results of monitoring of a CHF patient by an embodiment of the present invention. Analysis of the breathing related signal shown in FIG. 50 can be used to identify a CSR pattern by identifying the periodicity in the respiration motion amplitude and an apnea episode between each cycle. FIG. 52 shows the results of monitoring a CHF patient by an embodiment of the present invention and demodulating the respiratory signal to calculate the periodic breathing signal envelope. This is done by taking the absolute value of the breathing related signal and passing it through a low pass filter that filters out the breathing rate frequency, for example a low pass at a frequency of 0.1 Hz. The result—line 231—is the PB signal envelope. Line 232 shows the power spectrum of line 231. Peak 233 corresponds to the frequency of the periodic breathing—in this case a cycle time of about 50 seconds.

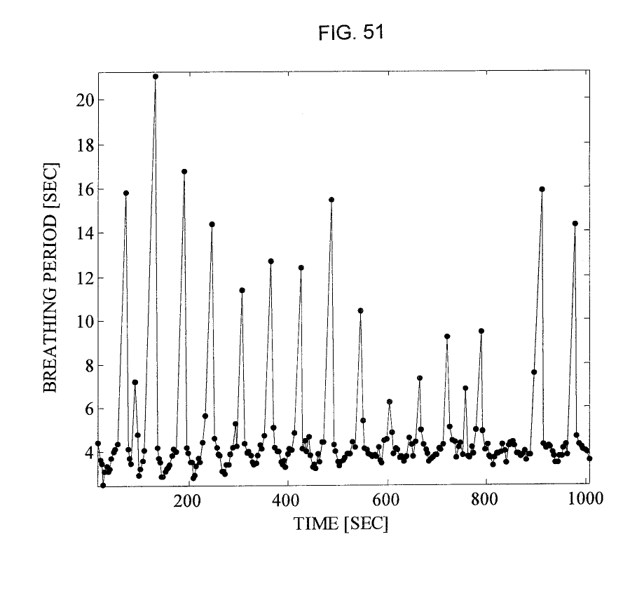

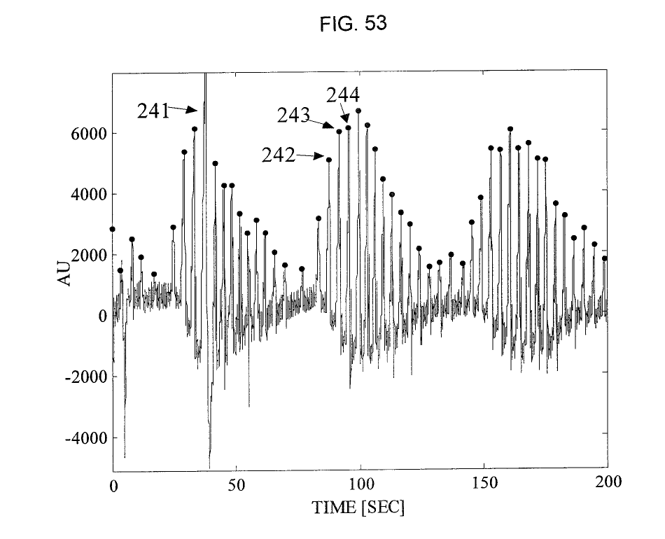

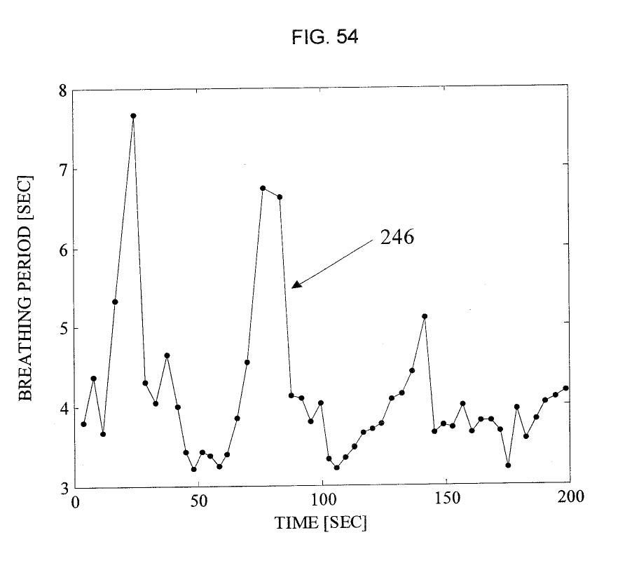

FIG. 51 shows the results of analysis of the data shown in FIG. 50 by pattern analysis module 16, in an embodiment of the present invention. In FIG. 51, each point represents the time between two successive breathing cycles. In one embodiment, pattern analysis module 16 compares the results shown in FIG. 51 to a defined CSR threshold—for example 10 seconds—each peak over that threshold during PB is then defined as a CSR event. The frequency of CSR events is an added parameter to the CHF score calculated by this embodiment. FIG. 53 shows an example of periodic breathing as measured while monitoring a CHF patient with an embodiment of the present invention. FIG. 54 shows the time between two successive breathing cycles calculated by an embodiment of the present invention on the signal shown in FIG. 53. In this case, line 246 does not have any points higher than the defined threshold of 10 seconds and therefore the system defines this as an event of PB and not CSR.

In one embodiment, system 10 may include a plurality of sensors, for example, a plurality of weight sensing sensors, placed under the mattress or mattress pad upon which patient 12 rests and the system may calculate a change of ratio of the average weight sensed by the sensors. A change in the weight ratio may indicate that patient 12 has changed posture for example, changed the angle of inclination during sleep. A change in the sleep angle indicates that a patient, for example, a CHF patient or a patient suffering from another physiological ailment, begins to feel decompensated. The sensing of this weight change may also be integrated into the clinical score and/or displayed separately to the patient and/or clinician.

Insomnia

In one embodiment, system 10 may be used to monitor subject 12 who is suspected of suffering from insomnia. For example, system 10 may monitor the duration a patient is in bed before falling into sleep, total duration of quiet sleep, the number of awakenings, sleep efficiency, and REM sleep duration and timing. An insomnia score may be calculated, for example, using one or more of the parameters used in the asthma score of hypoglycemia score discussed above, and presented to the subject or clinician. In one embodiment, system 10 may be further used to evaluate the effectiveness of different therapies to treat insomnia and the improvement that is gained by comparing the sleep quality parameters before and after treatment. In one embodiment, system 10 may detect the worsening of insomnia and indicate that a change or additional therapy may be required. In one embodiment, system 10 automatically activates or administers a therapy to treat insomnia when the sensors and analysis of system 10 deem such therapy appropriate.

Automated Response

In one embodiment, system 10 may identify the onset of an apnea or other physiological event and activate an appropriate treatment or therapy automatically, such as, CPAP or a change in body condition. For example, upon detecting the onset of apnea or other physiological event and/or upon predicting the oncoming apnea or other physiological event, system 10 may activate or administer an appropriated treatment or therapy within a short period of time (i.e., within seconds or minutes). In one embodiment, the activated treatment or therapy may be the activation of a device adapted to change the body and/or head position of subject 12, for example, so as to open up the airway in obstructive sleep apnea. For example, system 10 may include an inflatable pillow on which the patient sleeps which, when activated, inflates or deflates to vary the elevation of the head of subject 12 as desired. Upon detecting an oncoming or ongoing apnea or other physiological event, the pillow's air pressure level may be changed in order to change the patient's posture and prevent and/or stop the physiological event.

Heart Rate Standard Deviation

In one embodiment, system 10 monitors the heart rate of patient 12 during sleep and calculates the average heart rate for each minute of sleep time. Then the system calculates the standard deviation of the time series of minute by minute heart rate readings for that night. This standard deviation may then be used as a basis for monitoring one or more physiological conditions, such as, of asthma, COPD, and CHF deteriorations. For example, the ratio of the standard deviation versus the baseline for patient 12 may be calculated and uses as a metric or the ratio of the standard deviation to the baseline may be included in the clinical score of the patient and used to predict and monitor one or more physiological conditions, such as, asthma, COPD, and CHF deteriorations.

Although some embodiments described herein relate specifically to asthmatic episodes or CHF, the principles of the present invention may be applied, mutatis mutandis, to predicting and monitoring of one or more other respiratory and non-respiratory conditions that affect normal breathing patterns, such as chronic obstructive pulmonary disease (COPD), cystic fibrosis (CF), diabetes, a neurological disorder (e.g., epilepsy), and certain heart diseases in addition to CHF. For some applications, system 10 is configured to predict the onset of and/or monitor a migraine headache, such as by monitoring changes in respiration rate and/or heart rate, which are early indications of an approaching migraine. For some applications, system 10 is configured to monitor movement of the small bowel and/or colon movement, and to analyze such motion as an indication for gastrointestinal conditions. For example, system 10 may identify characteristic frequencies of gastrointestinal tract movement, such as by differentiating between signals generated by a sensor under the abdomen and a sensor under the lungs.

Techniques described herein may be practiced in combination with techniques described in one or more of the following applications, which are assigned to the assignee of the present patent application and are incorporated herein by reference: U.S. Provisional Patent Applications Nos. 60/674,382, 60/692,105, and 60/731,934, 60/784,799, and U.S. patent application Ser. No. 11/197,786, as well as the above-cited United States Patent Application Publication 2005/0192508 to Lange et al. and PCT Patent Publication WO 2005/074361.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-22. (canceled)

23. A method for detecting edema in a subject, the method comprising:
providing a plurality of mechanical sensors, each sensor adapted to sense a mechanical signal of a part of the body of the subject without contacting the subject;
sensing a plurality of mechanical signals from the plurality of sensors; and
analyzing the plurality of mechanical signals to determine the presence of edema.

24. The method as recited in claim 23, wherein said analyzing comprises detecting variation of the mechanical signal distribution of the subject with time to determine the presence of edema.

25. The method as recited in claim 23, wherein providing the plurality of mechanical sensors comprises providing a plurality of pressure sensors.

26. The method as recited in claim 23, wherein the method is practiced while the subject is asleep.

27. The method as recited in claim 23, wherein the method is practiced without requiring compliance of the subject.

28. A system for detecting edema in a subject, the system comprising:
a plurality of mechanical sensors, each sensor adapted to sense a mechanical signal of a part of the body of the subject without contacting the subject and produce a plurality of mechanical signals from the plurality of sensors; and
a signal analyzer adapted to analyze the plurality of mechanical signals to determine the presence of edema.

29. The system as recited in claim 28, wherein the analyzer is adapted to detect variation of the mechanical signal distribution of the subject with time to determine the presence of edema.

30. The system as recited in claim 28, wherein the plurality of mechanical sensors comprise a plurality of pressure sensors.

31. The system as recited in claim 28, wherein the system is adapted to be used while the subject is asleep.

32. The system as recited in claim 28, wherein the system is adapted to be used without requiring compliance of the subject.

33-116. (canceled)
